(19) United States
(12) Patent Application Publication
(10) Pub. No.: US 2025/0171500 A1
(43) Pub. Date: May 29, 2025

(54) CONFORMATIONALLY STABILIZED RSV PRE-FUSION F PROTEINS

(71) Applicants: Calder Biosciences Inc., New York, NY (US); The United States of America, as Represented by the Secretary, Department of He, Bethesda, MD (US)

(72) Inventors: Christopher Patrick Marshall, New York, NY (US); Jason Scott McLellan, Norwich, VT (US); Peter Joseph Alff, New York, NY (US); Claudio Bertuccioli, New York, NY (US); Roberto Mariani, San Diego, CA (US)

(21) Appl. No.: 18/675,033

(22) Filed: May 27, 2024

Related U.S. Application Data
(63) Continuation of application No. 17/332,901, filed on May 27, 2021, now Pat. No. 11,993,633.
Application No. 17/332,901 is a continuation of application No. 16/155,857, filed on Oct. 9, 2018, now abandoned.
Application No. 16/155,857 is a continuation of application No. 14/340,519, filed on Jul. 24, 2014, now Pat. No. 10,125,172.
(60) Provisional application No. 61/858,533, filed on Jul. 25, 2013.

(51) Int. Cl. C07K 14/005; A61K 39/00; A61K 39/12; A61K 39/155

(52) U.S. Cl. CPC C07K 14/005; A61K 39/12; A61K 39/155; A61K 2039/525; C12N 2760/18534

(57) ABSTRACT

In some embodiments, the present invention provides respiratory syncytial virus (RSV) F proteins, polypeptides and protein complexes that comprise one or more cross-links to stabilize the protein, polypeptide or protein complex in its pre-fusion conformation. In some embodiments the present invention provides RSV F proteins, polypeptides and protein complexes comprising one or more mutations to facilitate such cross-linking. In some embodiments the present invention provides compositions comprising such proteins, polypeptides or protein complexes, including vaccine compositions, and methods of making and using the same.

### CROSS-REFERENCE TO RELATED APPLICATIONS

This application is a continuation of U.S. patent application Ser. No. 17/332,901, filed on May 27, 2021, which was a continuation of U.S. patent application Ser. No. 16/155,857, filed on Oct. 9, 2018, which was a continuation of U.S. patent application Ser. No. 14/340,519, filed on Jul. 24, 2014, now U.S. Pat. No. 10,125,172, which claimed the benefit of priority of U.S. Provisional Patent Application No. 61/858,533, filed Jul. 25, 2013, the contents of each of which are hereby incorporated by reference.

### STATEMENT REGARDING FEDERALLY SPONSORED RESEARCH

This invention was made with government support under grant number 1R43AI112124-01A1 awarded by the National Institute of Allergy and Infectious Diseases (NIAID). The government has certain rights in the invention.

### SEQUENCE LISTING

The instant application contains a Sequence Listing which has been submitted electronically in XML file format and is hereby incorporated by reference in its entirety. Said XML copy, created on Feb. 13, 2025, is named Calder_007_US5_SL.xml and is 99,389 bytes in size.

### COPYRIGHT & INCORPORATION-BY-REFERENCE

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

For the purposes of only those jurisdictions that permit incorporation by reference, the text of all documents cited herein is hereby incorporated by reference in its entirety.

### BACKGROUND OF THE INVENTION

Each year, respiratory syncytial virus (RSV) infects 4-5 million children in the US, and is the leading cause of infant hospitalizations (˜150,000 hospitalizations). Globally, it accounts for 6.7% of deaths in infants less than 1 year old, second only to malaria. In addition, it poses a serious threat to other high-risk groups, including elderly and immuno-compromised subjects, where it results in approximately an additional 180,000 hospitalizations and 12,000 deaths in the US. There are no current frontline treatments for RSV, and the only currently approved prophylactic treatment for RSV is passive administration of the licensed monoclonal antibody Synagis (palivizumab), which recognizes the RSV fusion (F) protein, and reduces incidence of severe disease by only ˜50%. The high cost of prophylaxis with Synagis limits its use only to premature infants and infants less than 24 months old with congenital heart disease. For a review see Costello et al., “Targeting RSV with Vaccines and Small Molecule Drugs, Infectious Disorders,” Drug Targets, 2012, vol. 12, no. 2. The development of a more effective and, ideally, more cost-effective RSV vaccine would be of enormous value. Clinical evidence that RSV F protein-specific antibodies can protect against disease has prompted a concerted effort to identify additional and better monoclonal antibodies, and to develop a protective vaccine to address this significant unmet medical need.

### BRIEF SUMMARY OF THE INVENTION

Some aspects of the present invention are summarized below. Additional aspects are described in the Detailed Description of the Invention, the Examples, the Figures and the claims herein.

The RSV F protein is known to induce potent neutralizing antibodies that correlate with protection against RSV. Recently it has been shown that the pre-fusion conformation of the RSV F protein trimer (which may be referred to as “pre-fusion F” or “pre-F” herein) is the primary determinant of neutralizing activity in human sera. Also, the most potent neutralizing antibodies (nAbs) isolated to date specifically bind only to the pre-fusion conformation. However, soluble pre-F is highly unstable and readily transitions to the post-fusion conformation-limiting its usefulness as a vaccine immunogen. An RSV F protein stabilized in its pre-fusion (pre-F) conformation could be very valuable-providing a candidate RSV vaccine immunogen. Similarly, such a stabilized RSV pre-F protein could also be useful for the generation of antibodies, such as diagnostic and therapeutic antibodies. The crystal structure of the RSV F protein (bound to a potent nAb-D25) in its pre-fusion conformation was recently described. See McLellan et al., 2013, Science, 340, p. 1113-1117, the contents of which are hereby incorporated by reference in their entirety. Building on this work, the present inventors have performed extensive analysis of the structure of the RSV F protein and have developed a variety of novel design strategies and novel constructs to stabilize or “lock” the RSV F protein in its pre-F conformation.

In some embodiments the present invention provides RSV F polypeptides, proteins, and protein complexes, such as those that can be or are stabilized or “locked” in a pre-fusion conformation, for example using targeted cross-links, such as targeted di-tyrosine cross-links. The present invention also provides methods for making and using such RSV F polypeptides, proteins, and protein complexes.

In some embodiments, the present invention provides specific locations within the amino acid sequence of the RSV F protein at which, or between which, cross-links can be made in order to stabilize the RSV F protein in its pre-F conformation. In some embodiments, the cross-links are targeted di-tyrosine cross-links. Where di-tyrosine cross-links are used, the present invention provides specific amino acid residues (or pairs of amino acid residues) that either comprise a pre-existing tyrosine residue or can be or are mutated to a tyrosine residue such that di-tyrosine cross-links can be made.

In some embodiments, the present invention provides an isolated RSV F polypeptide, protein or protein complex comprising at least one di-tyrosine cross-link, wherein at least one tyrosine of the at least one di-tyrosine cross-links originates from a point mutation to tyrosine.

In some embodiments, the invention provides an isolated RSV F polypeptide, protein or protein complex having at least about 75% sequence identity to SEQ ID NO:1, 2, 3, 4, 5, 6, 7, 8, 9 or 10, or amino acid residues 1-513 thereof (comprising the RSV F ectodomain), wherein the protein polypeptide comprises at least one tyrosine residue that originates from a point mutation to tyrosine. In some such embodiments the RSV F polypeptides, proteins or protein complexes contain at least one di-tyrosine cross-link wherein at least one tyrosine residue of the at least one cross-link originates from a point mutation to tyrosine.

In some embodiments, the invention provides an isolated RSV F polypeptide having the amino acid sequence of SEQ ID NO:11, 12, 13, 14, 15, 16, 17, 18, 19, 20, 21, 22, 23, 24, 25, 26, 27, 28, 29, 30, 31 or 32, or amino acid residues 1-513 thereof (comprising the RSV F ectodomain). In some embodiments, the invention provides an isolated RSV F protein or polypeptide having at least about 75% sequence identity to SEQ ID NO:11, 12, 13, 14, 15, 16, 17, 18, 19, 20, 21, 22, 23, 24, 25, 26, 27, 28, 29, 30, 31 or 32, or amino acid residues 1-513 thereof (comprising the RSV F ectodomain). In some such embodiments the RSV F polypeptides, proteins or protein complexes contain at least one di-tyrosine cross-link wherein at least one tyrosine residue of the at least one cross-link originates from a point mutation to tyrosine.

In some embodiments, where di-tyrosine cross-links are present, the di-tyrosine cross-link comprises two pre-existing tyrosine residues. In some embodiments, the di-tyrosine cross-link comprises a pre-existing tyrosine cross-linked to a tyrosine originating from a point mutation to tyrosine. In some embodiments, the di-tyrosine cross-link comprises two tyrosines originating from point mutations to tyrosine. In some embodiments, the di-tyrosine cross-link comprises an intra-protomer bond, an inter-protomer bond, an intra-molecular bond, an inter-molecular bond, or any combination thereof. In some embodiments, the di-tyrosine cross-link comprises a bond within or between a RSV F protein F1 polypeptide and a RSV F protein F2 polypeptide. In some embodiments, the point mutation to tyrosine is located at one or more amino acid positions selected from the group consisting of amino acid positions: 77, 88, 97, 147, 150, 155, 159, 183, 185, 187, 220, 222, 223, 226, 255, 427 or 469 of SEQ ID NO: 1 or SEQ ID NO: 4, or any amino acid position that corresponds to one of such amino acid positions in another RSV F amino acid sequence or ectodomain thereof.

In some embodiments, the invention provides an isolated RSV F polypeptide, protein or protein complex having at least about 75% sequence identity to SEQ ID NO:1, 2, 3, 4, 5, 6, 7, 8, 9 or 10, wherein the polypeptide comprises at least one di-tyrosine cross-link, wherein one or both tyrosines of the cross-link originates from a point mutation to tyrosine, and wherein the cross-links are located between one or more paired tyrosine amino acid residues located at amino acid positions: 147 and 286; 198 and 220; 198 and 222; 198 and 223; 198 and 226; 33 and 469; 77 and 222; 88 and 255; 97 and 159; 183 and 427; 185 and 427; or 187 and 427. In some embodiments, the RSV F polypeptide, protein, or protein complex comprises more than one di-tyrosine cross-link. In some embodiments, the RSV F polypeptide, protein, or protein complex comprises two, three, four, five or more di-tyrosine cross-links. In some embodiments, the RSV F polypeptide, protein, or protein complex comprises di-tyrosine cross-links at paired tyrosine amino acid residues located at amino acid positions 77 and 222, and 33 and 469.

In some embodiments, the RSV F polypeptides, proteins, or protein complexes of the invention further comprise one or more additional cross-links, such as disulfide bonds. In such embodiments, at least one cysteine of the one or more disulfide bonds originates from a point mutation to cysteine. In some embodiments, the RSV F polypeptides, proteins, or protein complexes further comprise cavity-filling hydrophobic amino acid substitutions. In some embodiments, the RSV F polypeptides, proteins, or protein complexes further comprises a trimerization domain. In such embodiments, the trimerization domain is a foldon domain.

In some embodiments, the RSV F polypeptides, proteins, or protein complexes of the invention are capable of eliciting a protective immune response in a subject and/or eliciting production of RSV-specific neutralizing antibodies in a subject. In some embodiments, the the RSV F polypeptides, proteins, or protein complexes of the invention comprise at least one antigenic site capable of binding a neutralizing antibody, for example, the antigenic site ø.

In some embodiments, the invention provides compositions (such as pharmaceutical compositions and/or vaccine compositions) comprising one or more RSV F polypeptides, proteins, or protein complexes of the invention. In some embodiments, such compositions comprise an adjuvant, a carrier, an immunostimulatory agent, or any combination thereof. In some embodiments, the composition is, or forms part of, a vaccine for respiratory syncytial virus. In some embodiments, the invention provides a method of vaccinating a subject against RSV, the method comprising administering an effective amount of a composition comprising one or more of the RSV F polypeptides, proteins, or protein complexes of the invention to a subject. In some embodiments, the subject is a human of less than 24 months in age or a human of greater than 50 years in age. In some embodiments, the administering comprises a single immunization. In some embodiments, a method of the invention further comprises administering to a subject a pharmaceutical composition comprising one or more RSV F polypeptides, proteins, or protein complexes of the invention so as to treat or prevent an RSV infection in the subject. In some embodiments, the invention provides a medicament for inducing an immune response in a subject, comprising one or more the RSV F polypeptides, proteins, or protein complexes of the invention. In some embodiments, the medicament is a vaccine.

In some embodiments, the invention provides a method of making a RSV vaccine immunogen, comprising (a) identifying or obtaining a RSV F polypeptide, protein or protein complex in a pre-fusion conformation; (b) selecting one or more regions in the RSV F polypeptide, protein or protein complex where the introduction of one or more cross-links (such as di-tyrosine cross-links) could stabilize the pre-fusion conformation; (c) introducing into the RSV F protein one or more cross-links (such as di-tyrosine cross-links) at one or more of the regions selected in step (b) to form an engineered RSV F polypeptide, protein or protein complex; and (d) determining if the engineered RSV F polypeptide, protein or protein complex has one or more properties selected from the group consisting of: (i) enhanced ability bind to a neutralizing antibody, (ii) enhanced ability bind to a broadly neutralizing, (iii) enhanced ability bind to and activate B cell receptors, (iv) enhanced ability to elicit an antibody response in an animal, (v) enhanced ability to elicit a protective antibody response in an animal, (vi) enhanced ability to elicit production of neutralizing antibodies in an animal, (vii) enhanced ability to elicit production of broadly neutralizing antibodies in an animal, (viii) enhanced ability to elicit a protective immune response in an animal, and (ix) enhanced ability to bind to and elicit production of antibodies that recognize quaternary neutralizing epitopes in an animal, wherein if the engineered RSV F polypeptide, protein or protein complex has one or more properties i. to ix., the engineered RSV F polypeptide, protein or protein complex is a RSV vaccine immunogen candidate. In some such embodiments, step (d) comprises performing one or more assays to assess the ability of the engineered RSV F protein to bind to a neutralizing antibody, bind to a broadly neutralizing antibody, bind to and activate a B cell receptors elicit an antibody response in an animal, elicit a protective antibody response in an animal, elicit production of neutralizing antibodies in an animal, elicit production of broadly neutralizing antibodies in an animal, elicit a protective immune response in an animal, and/or elicit production of antibodies that recognize quaternary neutralizing epitopes in an animal. In some embodiments, where di-tyrosine cross-links are used, at least one tyrosine of the one or more di-tyrosine cross-links introduced in step (c) originates from a point mutation to tyrosine. In some embodiments, the method further comprises, prior to step (c), introducing into the RSV F protein one or more point mutations to tyrosine at one or more of the regions selected in step (b).

These and other embodiments of the present invention are described throughout the present patent specification.

### BRIEF DESCRIPTION OF THE DRAWINGS

FIGS. 1A-1B. Amino acid sequences of (A) soluble RSV F protein from RSV subtype A (WT) (SEQ ID NO:1), and (B) full-length RSV F protein from RSV subtype A (SEQ ID NO: 2) (Accession No. AHJ60043.1). Amino acid residues 1-513 of both sequences are the core sequences of the RSV F protein that are common to both the soluble and membrane-bound (full-length) forms. The C-terminal sequences from amino acid residue 514 onwards in SEQ ID NO. 1 comprise a foldon trimerization domain followed by a thrombin cleavage site, 6×His-tag (SEQ ID NO: 46), and a strep tag. The C-terminal sequences from amino acid residue 514 onwards in SEQ ID NO. 2 comprise the endogenous RSV F protein sequence containing the transmembrane region and cytoplasmic tail.

FIGS. 2A-2B. Amino acid sequences of (A) soluble RSV F protein from RSV subtype B (SEQ ID NO:3), and (B) full-length RSV F protein from RSV subtype B (SEQ ID NO: 4) (Accession No. AHL84194). Amino acid residues 1-513 of both sequences are the core sequences of the RSV F protein that are common to both the soluble and membrane-bound (full-length) forms. The C-terminal sequences from amino acid residue 514 onwards in SEQ ID NO. 3 comprise a foldon trimerization domain followed by a thrombin cleavage site, 6×His-tag (SEQ ID NO: 46), and a strep tag. The C-terminal sequences from amino acid residue 514 onwards in SEQ ID NO. 4 comprise the endogenous RSV F protein sequence containing the transmembrane region and cytoplasmic tail.

FIGS. 3A-3B. Amino acid sequences of (A) soluble DS-Cav1 modified RSV F protein (SEQ ID NO:5), and (B) full-length DS-Cav1 modified RSV F protein (SEQ ID NO:6) (McLellan et al. (2013) Science 342:592-598). Amino acid residues 1-513 of both sequences are the core sequences of the RSV F protein that are common to both the soluble and membrane-bound (full-length) forms. The C-terminal sequences from amino acid residue 514 onwards in SEQ ID NO. 5 comprise a foldon trimerization domain followed by a thrombin cleavage site, 6×His-tag (SEQ ID NO: 46), and a strep tag. The C-terminal sequences from amino acid residue 514 onwards in SEQ ID NO. 6 comprise the endogenous RSV F protein sequence containing the transmembrane region and cytoplasmic tail.

FIGS. 4A-4B. Amino acid sequences of (A) soluble Cav1 modified RSV F protein (SEQ ID NO:7), and (B) full-length Cav1 modified RSV F protein sequence (SEQ ID NO: 8) (McLellan et al. (2013) Science 342:592-598).). Amino acid residues 1-513 of both sequences are the core sequences of the RSV F protein that are common to both the soluble and membrane-bound (full-length) forms. The C-terminal sequences from amino acid residue 514 onwards in SEQ ID NO. 7 comprise a foldon trimerization domain followed by a thrombin cleavage site, 6×His-tag (SEQ ID NO: 46), and a strep tag. The C-terminal sequences from amino acid residue 514 onwards in SEQ ID NO. 8 comprise the endogenous RSV F protein sequence containing the transmembrane region and cytoplasmic tail.

FIGS. 5A-5B. Amino acid sequences of (A) soluble DS modified RSV F protein (SEQ ID NO:9), and (B) full-length DS modified RSV F protein sequence (SEQ ID NO:10) (McLellan et al. (2013) Science 342:592-598).). Amino acid residues 1-513 of both sequences are the core sequences of the RSV F protein that are common to both the soluble and membrane-bound (full-length) forms. The C-terminal sequences from amino acid residue 514 onwards in SEQ ID NO. 9 comprise a foldon trimerization domain followed by a thrombin cleavage site, 6×His-tag (SEQ ID NO: 46), and a strep tag. The C-terminal sequences from amino acid residue 514 onwards in SEQ ID NO. 10 comprise the endogenous RSV F protein sequence containing the transmembrane region and cytoplasmic tail.

FIG. 6. Sequence alignment of RSV F proteins from RSV subtype A (SEQ ID NO: 1-identified as “RSV_F_WT” in the figure) and subtype B (SEQ ID NO:4). Amino acid residues shown in bold and underlined in the subtype A sequence indicate sites that can be targeted for di-tyrosine cross-linking either as single or double mutants. The equivalent sites in subtype B are also shown in boxes. The designated positions targeted for di-tyrosine cross-linking are 100% conserved between RSV subtypes A and B. Amino acid residues 1-513 of both sequences are the core sequences of the RSV F protein. The C-terminal sequences from amino acid residue 514 onwards in SEQ ID NO. 1 comprise a foldon trimerization domain followed by a thrombin cleavage site, 6×His-tag (SEQ ID NO: 46), and a strep tag. The C-terminal sequences from amino acid residue 514 onwards in SEQ ID NO. 4 comprise the endogenous RSV F protein sequence containing the transmembrane region and cytoplasmic tail.

FIG. 7. Sequence alignment of DS-Cav1 RSV F protein (SEQ ID NO:5), and RSV F proteins from RSV subtype A (SEQ ID NO:1) and subtype B (SEQ ID NO:4). The C-terminal sequences shown in italics in the DS-Cav1 and RSV subtype A sequences (residues 514-568) contain the exogenous Foldon trimerization domain followed by a thrombin cleavage site, 6×His-tag (SEQ ID NO: 46) and a strep tag. The C-terminal sequence shown in bold and underlined in the RSV subtype B sequence (residues 514-574) is the endogenous F protein sequence containing the transmembrane region and cytoplasmic tail.

FIG. 8. Amino acid sequence of a modified soluble RSV F protein (subtype A) comprising a to-tyrosine mutation at position 147 (A147Y) (SEQ ID NO:11). Amino acid residues 1-513 are the core sequences of the RSV F protein. The C-terminal sequences from amino acid residue 514 onwards comprise a foldon trimerization domain followed by a thrombin cleavage site, 6×His-tag (SEQ ID NO: 46), and a strep tag.

FIG. 9. Amino acid sequence of a modified soluble RSV F protein (subtype A) comprising a to-tyrosine mutation at position 220 (V220Y) (SEQ ID NO:12). Amino acid residues 1-513 are the core sequences of the RSV F protein. The C-terminal sequences from amino acid residue 514 onwards comprise a foldon trimerization domain followed by a thrombin cleavage site, 6×His-tag (SEQ ID NO: 46), and a strep tag.

FIG. 10. Amino acid sequence of a modified soluble RSV F protein (subtype A) comprising a to-tyrosine mutation at position 222 (E222Y) (SEQ ID NO:13). Amino acid residues 1-513 are the core sequences of the RSV F protein. The C-terminal sequences from amino acid residue 514 onwards comprise a foldon trimerization domain followed by a thrombin cleavage site, 6×His-tag (SEQ ID NO: 46), and a strep tag.

FIG. 11. Amino acid sequence of a modified soluble RSV F protein (subtype A) comprising a to-tyrosine mutation at position 223 (F223Y) (SEQ ID NO:14). Amino acid residues 1-513 are the core sequences of the RSV F protein. The C-terminal sequences from amino acid residue 514 onwards comprise a foldon trimerization domain followed by a thrombin cleavage site, 6×His-tag (SEQ ID NO: 46), and a strep tag.

FIG. 12. Amino acid sequence of a modified soluble RSV F protein (subtype A) comprising a to-tyrosine mutation at position 226 (K226Y) (SEQ ID NO:15). Amino acid residues 1-513 are the core sequences of the RSV F protein. The C-terminal sequences from amino acid residue 514 onwards comprise a foldon trimerization domain followed by a thrombin cleavage site, 6×His-tag (SEQ ID NO: 46), and a strep tag.

FIG. 13. Amino acid sequence of a modified soluble RSV F protein (subtype A) comprising a to-tyrosine mutation at position 469 (V469Y) (SEQ ID NO:16). Amino acid residues 1-513 are the core sequences of the RSV F protein. The C-terminal sequences from amino acid residue 514 onwards comprise a foldon trimerization domain followed by a thrombin cleavage site, 6×His-tag (SEQ ID NO: 46), and a strep tag.

FIG. 14. Amino acid sequence of a modified soluble RSV F protein (subtype A) comprising to-tyrosine mutations at positions 77 (K77Y) and 222 (E222Y) (SEQ ID NO:17). Amino acid residues 1-513 are the core sequences of the RSV F protein. The C-terminal sequences from amino acid residue 514 onwards comprise a foldon trimerization domain followed by a thrombin cleavage site, 6×His-tag (SEQ ID NO: 46), and a strep tag.

FIG. 15. Amino acid sequence of a modified soluble RSV F protein (subtype A) comprising to-tyrosine mutations at positions 88 (N88Y) and 255 (S255Y) (SEQ ID NO:18). Amino acid residues 1-513 are the core sequences of the RSV F protein. The C-terminal sequences from amino acid residue 514 onwards comprise a foldon trimerization domain followed by a thrombin cleavage site, 6×His-tag (SEQ ID NO: 46), and a strep tag.

FIG. 16. Amino acid sequence of a modified soluble RSV F protein (subtype A) comprising to-tyrosine mutations at positions 97 (M97Y) and 159 (H159Y) (SEQ ID NO:19). Amino acid residues 1-513 are the core sequences of the RSV F protein. The C-terminal sequences from amino acid residue 514 onwards comprise a foldon trimerization domain followed by a thrombin cleavage site, 6×His-tag (SEQ ID NO: 46), and a strep tag.

FIG. 17. Amino acid sequence of a modified soluble RSV F protein (subtype A) comprising to-tyrosine mutations at positions 185 (V185Y) and 427 (K427Y) (SEQ ID NO:20). Amino acid residues 1-513 are the core sequences of the RSV F protein. The C-terminal sequences from amino acid residue 514 onwards comprise a foldon trimerization domain followed by a thrombin cleavage site, 6×His-tag (SEQ ID NO: 46), and a strep tag.

FIG. 18. Amino acid sequence of a modified soluble RSV F protein (subtype A) comprising to-tyrosine mutations at positions 187 (V187Y) and 427 (K427Y) (SEQ ID NO:21). Amino acid residues 1-513 are the core sequences of the RSV F protein. The C-terminal sequences from amino acid residue 514 onwards comprise a foldon trimerization domain followed by a thrombin cleavage site, 6×His-tag (SEQ ID NO: 46), and a strep tag.

FIG. 19. Amino acid sequence of a modified soluble RSV F protein (subtype A) comprising to tyrosine mutations at positions 183 (N183Y) and 427 (K427Y) (SEQ ID NO:22). Amino acid residues 1-513 are the core sequences of the RSV F protein. The C-terminal sequences from amino acid residue 514 onwards comprise a foldon trimerization domain followed by a thrombin cleavage site, 6×His-tag (SEQ ID NO: 46), and a strep tag.

FIG. 20A-20C. Sequence alignment of soluble RSV F protein subtype A (SEQ ID NO: 1) and examples of modified RSV F proteins derived therefrom (SEQ ID NOS: 11-21) comprising single or double to-tyrosine mutations. Tyrosines in boxes are introduced into the WT subtype A sequence. Where two new tyrosines are introduced into the same sequence, they are typically intended to cross-link with each other. Where only a single tyrosine is introduced, that tyrosine is typically expected to cross-link with an endogenous or pre-existing tyrosine in that sequence which is shown in bold and underlined. Amino acid residues 1-513 of each sequence are the core sequences of the RSV F protein. The C-terminal sequences from amino acid residue 514 onwards comprise a foldon trimerization domain followed by a thrombin cleavage site, 6×His-tag (SEQ ID NO: 46), and a strep tag.

FIG. 21. Amino acid sequence of a modified soluble RSV F protein (DS-Cav1) comprising a to-tyrosine mutation at position 147 (A147Y) (SEQ ID NO:23). Amino acid residues 1-513 are the core sequences of the RSV F protein. The C-terminal sequences from amino acid residue 514 onwards comprise a foldon trimerization domain followed by a thrombin cleavage site, 6×His-tag (SEQ ID NO: 46), and a strep tag.

FIG. 22. Amino acid sequence of a modified soluble RSV F protein (DS-Cav1) comprising a to-tyrosine mutation at position 220 (V220Y) (SEQ ID NO:24). Amino acid residues 1-513 are the core sequences of the RSV F protein. The C-terminal sequences from amino acid residue 514 onwards comprise a foldon trimerization domain followed by a thrombin cleavage site, 6×His-tag (SEQ ID NO: 46), and a strep tag.

FIG. 23. Amino acid sequence of a modified soluble RSV F protein (DS-Cav1) comprising a to-tyrosine mutation at position 222 (E222Y) (SEQ ID NO:25). Amino acid residues 1-513 are the core sequences of the RSV F protein. The C-terminal sequences from amino acid residue 514 onwards comprise a foldon trimerization domain followed by a thrombin cleavage site, 6×His-tag (SEQ ID NO: 46), and a strep tag.

FIG. 24. Amino acid sequence of a modified soluble RSV F protein (DS-Cav1) comprising a to-tyrosine mutation at position 223 (F223Y) (SEQ ID NO:26). Amino acid residues 1-513 are the core sequences of the RSV F protein. The C-terminal sequences from amino acid residue 514 onwards comprise a foldon trimerization domain followed by a thrombin cleavage site, 6×His-tag (SEQ ID NO: 46), and a strep tag.

FIG. 25. Amino acid sequence of a modified soluble RSV F protein (DS-Cav1) comprising a to-tyrosine mutation at position 226 (K226Y) (SEQ ID NO:27). Amino acid residues 1-513 are the core sequences of the RSV F protein. The C-terminal sequences from amino acid residue 514 onwards comprise a foldon trimerization domain followed by a thrombin cleavage site, 6×His-tag (SEQ ID NO: 46), and a strep tag.

FIG. 26. Amino acid sequence of a modified soluble RSV F protein (DS-Cav1) comprising a to-tyrosine mutation at position 469 (V469Y) (SEQ ID NO:28). Amino acid residues 1-513 are the core sequences of the RSV F protein. The C-terminal sequences from amino acid residue 514 onwards comprise a foldon trimerization domain followed by a thrombin cleavage site, 6×His-tag (SEQ ID NO: 46), and a strep tag.

FIG. 27. Amino acid sequence of a modified soluble RSV F protein (subtype A) comprising to-tyrosine mutations at positions 222 (E222Y) and 469 (V469Y) (SEQ ID NO:29) designed to facilitate the formation of multiple di-tyrosine cross-links. Amino acid residues 1-513 are the core sequences of the RSV F protein. The C-terminal sequences from amino acid residue 514 onwards comprise a foldon trimerization domain followed by a thrombin cleavage site, 6×His-tag (SEQ ID NO: 46), and a strep tag.

FIG. 28. Amino acid sequence of a modified soluble RSV F protein (subtype A) comprising to-tyrosine mutations at positions 226 (K226Y) and 469 (V469Y) (SEQ ID NO:30) designed to facilitate the formation of multiple di-tyrosine cross-links. Amino acid residues 1-513 are the core sequences of the RSV F protein. The C-terminal sequences from amino acid residue 514 onwards comprise a foldon trimerization domain followed by a thrombin cleavage site, 6×His-tag (SEQ ID NO: 46), and a strep tag.

FIG. 29. Amino acid sequence of a modified soluble RSV F protein (DS-Cav1) comprising to-tyrosine mutations at positions 222 (E222Y) and 469 (V469Y) (SEQ ID NO:31) designed to facilitate the formation of multiple di-tyrosine cross-links. Amino acid residues 1-513 are the core sequences of the RSV F protein. The C-terminal sequences from amino acid residue 514 onwards comprise a foldon trimerization domain followed by a thrombin cleavage site, 6×His-tag (SEQ ID NO: 46), and a strep tag.

FIG. 30. Amino acid sequence of a modified soluble RSV F protein (DS-Cav1) comprising to-tyrosine mutations at positions 226 (K226Y) and 469 (V469Y) (SEQ ID NO:32) designed to facilitate the formation of multiple di-tyrosine cross-links. Amino acid residues 1-513 are the core sequences of the RSV F protein. The C-terminal sequences from amino acid residue 514 onwards comprise a foldon trimerization domain followed by a thrombin cleavage site, 6×His-tag (SEQ ID NO: 46), and a strep tag.

FIGS. 31A-31B. Ribbon diagrams depicting examples of two targeted positions in the RSV prefusion F protein trimer where intra-protomeric and intra-protomeric di-tyrosine cross-links can be introduced. (A) Side-view (and enlargement) of an engineered F1-F1 inter-protomeric di-tyrosine bond between a tyrosine introduced at position 185 (V185Y) and a tyrosine introduced at position 427 (K427Y) (SEQ ID NO:20). (B) Top-down view (and enlargement) of an engineered F1-F1 intra-protomeric di-tyrosine bond between an endogenous tyrosine at position 198 (198Y) and a tyrosine introduced at position 222 (E222Y) (SEQ ID NO: 13). Targeted tyrosine residues are depicted in square boxes.

FIG. 32. Alignment of the RSV type A F protein sequence (SEQ ID NO: 47), and of the prefusion and post-fusion secondary structures. Cylinders and arrows below the sequence represent α-helices and β-strands, respectively. An “X” below the amino acid sequence indicates where the structure is disordered or missing. Gray shadowing indicates the position of residues that move more than 5 Å in the transition from the prefusion to postfusion conformation. Black triangles indicate sites of N-linked glycosylation, text and lines above the amino acid sequence indicate the antigenic sites, and arrows indicate the position of the furin cleavage sites. Figure from Mclellan et al., 2013, Science 340:1113-1117, supplementary materials.

FIG. 33. Illustrative nucleic acid construct for expression of RSV F protein in mammalian cells.

FIG. 34. Targeted di-tyrosine cross-linking of RSV F protein. HEK 293 cells were transfected with vectors expressing RSV-F variants that enabling F₁-F₁ intra-protomer DT-cross-links (E222Y (SEQ ID NO: 13) and K226Y (SEQ ID NO:15)), F₁-F₂ intra-protomer DT-cross-links (V469Y (SEQ ID NO:16) and N88Y-S255Y (SEQ ID NO:18)), or F₁-F₁ intermolecular DT-cross-links (V185Y-K427Y (SEQ ID NO:20)). Background-subtracted fluorescence intensity values are shown. (INT) intensity, (DT) di-tyrosine, (−) and (+) indicate before and after application of the di-tyrosine cross-linking technology, respectively.

FIGS. 35A-35B. Di-tyrosine cross-linking stabilizes key epitope on RSV prefusion F protein. HEK 293 cells were transfected with constructs expressing the wild-type (WT) RSV-F or a variant containing the K226Y substitution. (A) 72 h post transfection, supernatents were cross-linked (DT) or left uncross-linked and total protein was measured by ELISA using a high-affinity human anti-hRSV antibody (100 ng/ml in PBS) that recognizes both pre- and post-fusion forms of RSV-F. (B) Following storage at 4 degrees C. for 16 days presentation of site ø was measured by ELISA using a preF specific human monoclonal antibody (2 μg/ml in PBS) that recognizes site ø.

FIGS. 36A-36B. Sequence alignment of nucleotide sequences encoding the RSV F protein (full-length—with transmembrane and cytoplasmic domains) from RSV subtype A (SEQ ID NO:33—identified as “RSV_F_WT_Subtype_A” in the figure) and RSV subtype B (SEQ ID NO:34—identified as “RSV_F_WT_Subtype_B” in the figure). Nucleotides 1-1539 encode amino acid residues 1-513, which are the core ectodomain sequences of the RSV F protein. Nucleotides 1540 onwards comprise sequences that encode the endogenous RSV F transmembrane region and cytoplasmic tail.

FIG. 37. Nucleotide sequences encoding the RSV F protein (full-length—with transmembrane and cytoplasmic domains) from RSV subtype A that has been codon optimized for expression in human cells (SEQ ID NO:35). Nucleotides 1-1539 encode amino acid residues 1-513, which are the core ectodomain sequences of the RSV F protein. Nucleotides 1540 onwards comprise sequences that encode the endogenous RSV F transmembrane region and cytoplasmic tail.

FIG. 38. Nucleotide sequences encoding the RSV F protein (full-length—with transmembrane and cytoplasmic domains) from RSV subtype A that has been codon optimized for expression in hamster cells (such as CHO cells) (SEQ ID NO:36). Nucleotides 1-1539 encode amino acid residues 1-513, which are the core ectodomain sequences of the RSV F protein. Nucleotides 1540 onwards comprise sequences that encode the endogenous RSV F transmembrane region and cytoplasmic tail.

FIG. 39. Nucleotide sequences encoding the RSV F protein (full-length—with transmembrane and cytoplasmic domains) from RSV subtype A that has been codon optimized for expression in insect cells (such as SF9 insect cells) (SEQ ID NO:37). Nucleotides 1-1539 encode amino acid residues 1-513, which are the core ectodomain sequences of the RSV F protein. Nucleotides 1540 onwards comprise sequences that encode the endogenous RSV F transmembrane region and cytoplasmic tail.

FIG. 40. Nucleotide sequences encoding the RSV F protein (full-length—with transmembrane and cytoplasmic domains) from RSV subtype A that has been codon optimized for expression in mouse cells (SEQ ID NO:38). Nucleotides 1-1539 encode amino acid residues 1-513, which are the core ectodomain sequences of the RSV F protein. Nucleotides 1540 onwards comprise sequences that encode the endogenous RSV F transmembrane region and cytoplasmic tail.

FIG. 41. Nucleotide sequences encoding the RSV F protein (full-length—with transmembrane and cytoplasmic domains) from RSV subtype B that has been codon optimized for expression in human cells (SEQ ID NO:39). Nucleotides 1-1539 encode amino acid residues 1-513, which are the core ectodomain sequences of the RSV F protein. Nucleotides 1540 onwards comprise sequences that encode the endogenous RSV F transmembrane region and cytoplasmic tail.

FIG. 42. Nucleotide sequences encoding the RSV F protein (full-length—with transmembrane and cytoplasmic domains) from RSV subtype B that has been codon optimized for expression in hamster cells (such as CHO cells) (SEQ ID NO:40). Nucleotides 1-1539 encode amino acid residues 1-513, which are the core ectodomain sequences of the RSV F protein. Nucleotides 1540 onwards comprise sequences that encode the endogenous RSV F transmembrane region and cytoplasmic tail.

FIG. 43. Nucleotide sequences encoding the RSV F protein (full-length—with transmembrane and cytoplasmic domains) from RSV subtype B that has been codon optimized for expression in insect cells (such as SF9 insect cells) (SEQ ID NO:41). Nucleotides 1-1539 encode amino acid residues 1-513, which are the core ectodomain sequences of the RSV F protein. Nucleotides 1540 onwards comprise sequences that encode the endogenous RSV F transmembrane region and cytoplasmic tail.

FIG. 44. Nucleotide sequences encoding the RSV F protein (full-length—with transmembrane and cytoplasmic domains) from RSV subtype B that has been codon optimized for expression in mouse cells (SEQ ID NO:42). Nucleotides 1-1539 encode amino acid residues 1-513, which are the core ectodomain sequences of the RSV F protein. Nucleotides 1540 onwards comprise sequences that encode the endogenous RSV F transmembrane region and cytoplasmic tail.

FIG. 45. Nucleotide sequences encoding the RSV F protein (full-length—with transmembrane and cytoplasmic domains) from RSV subtype A that has been codon optimized for expression in human cells and also comprises DS-CAV1 mutations (SEQ ID NO:43). The mutations are shown in bold and with boxes surrounding the mutated codons. Nucleotides 1-1539 encode amino acid residues 1-513, which are the core ectodomain sequences of the RSV F protein. Nucleotides 1540 onwards comprise sequences that encode the endogenous RSV F transmembrane region and cytoplasmic tail.

FIG. 46. Nucleotide sequences encoding the RSV F protein (full-length—with transmembrane and cytoplasmic domains) from RSV subtype A that has been codon optimized for expression in human cells and also comprises DS mutations (SEQ ID NO:44). The mutations are shown in bold and with boxes surrounding the mutated codons. Nucleotides 1-1539 encode amino acid residues 1-513, which are the core ectodomain sequences of the RSV F protein. Nucleotides 1540 onwards comprise sequences that encode the endogenous RSV F transmembrane region and cytoplasmic tail.

FIG. 47. Nucleotide sequences encoding the RSV F protein (full-length—with transmembrane and cytoplasmic domains) from RSV subtype A that has been codon optimized for expression in human cells and also comprises CAV1 mutations (SEQ ID NO:45). The mutations are shown in bold and with boxes surrounding the mutated codons. Nucleotides 1-1539 encode amino acid residues 1-513, which are the core ectodomain sequences of the RSV F protein. Nucleotides 1540 onwards comprise sequences that encode the endogenous RSV F transmembrane region and cytoplasmic tail.

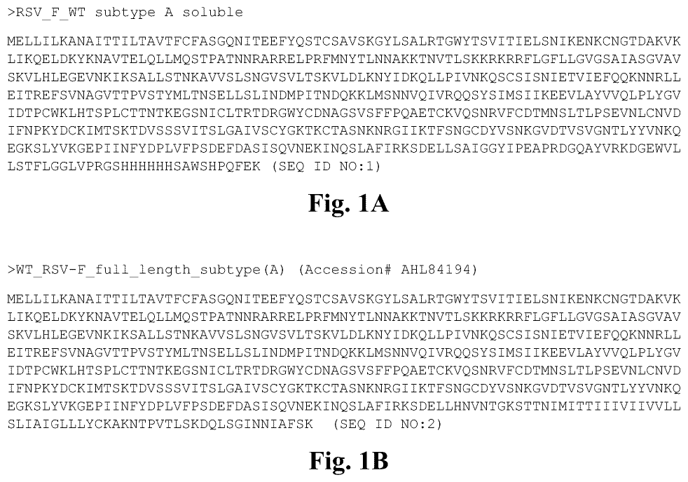

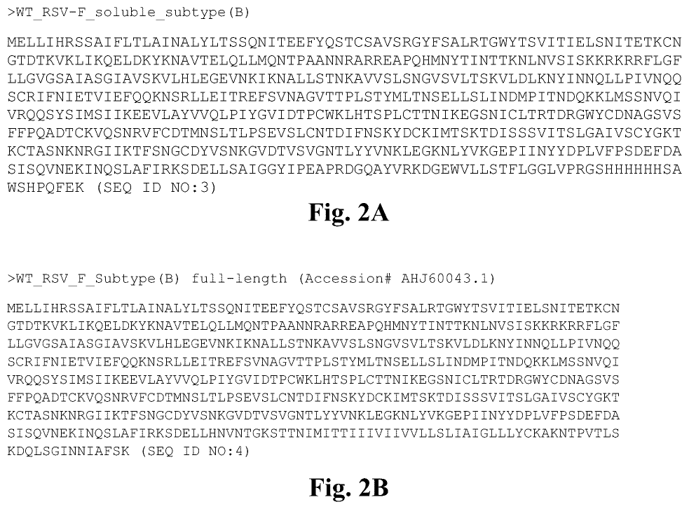

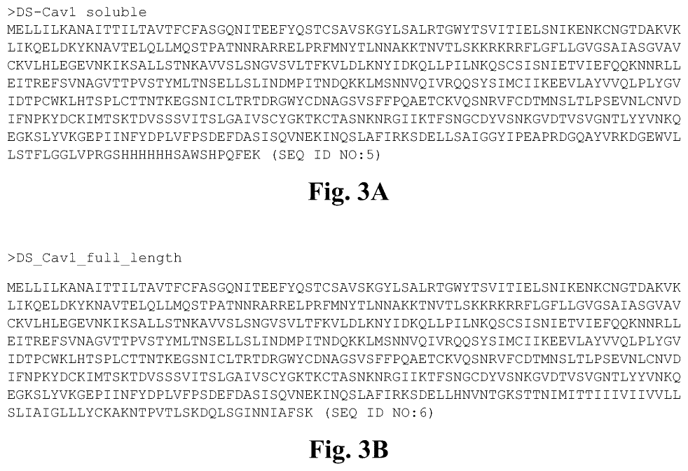

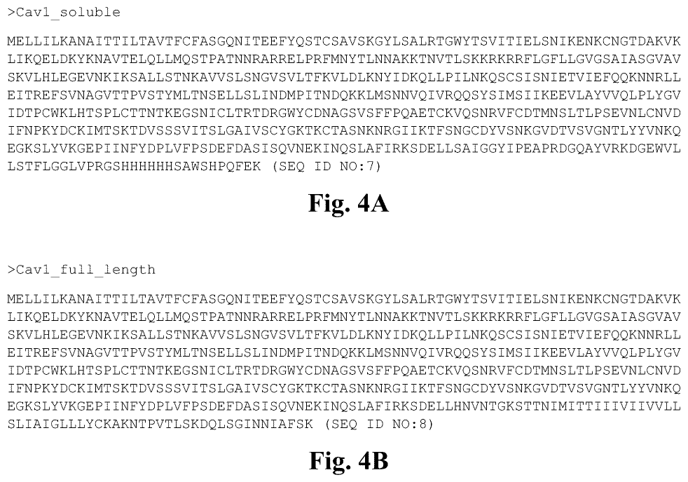

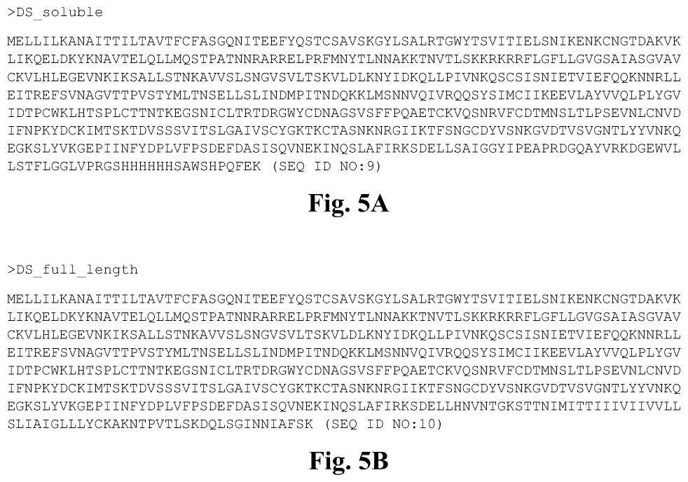

### DETAILED DESCRIPTION OF THE INVENTION

The present invention provides, in part, RSV F polypeptides, proteins and protein complexes, such as those that can be or are stabilized in a pre-fusion conformation, methods of making such polypeptides, proteins and protein complexes, compositions (such as pharmaceutical compositions and vaccine compositions) comprising such polypeptides, proteins and protein complexes, and methods of use of such polypeptides, proteins and protein complexes, for example in vaccination methods, therapeutic methods and other methods. In some embodiments, the RSV polypeptides, proteins and protein complexes may be useful as conformationally-specific immunogens, for example in RSV vaccines.

### Definitions and Abbreviations

As used in the present specification the terms “about” and “approximately,” when used in relation to numerical values, mean within + or −20% of the stated value.

As used herein the terms “protein” and “polypeptide” are used interchangeably, unless otherwise stated. As used herein the term “protein complex” refers to an assembly of two or more proteins or protein subunits, such as two or more monomers or protomers. Unless otherwise stated, all description herein that relates to proteins applies equally to protein complexes, and vice versa.

As used herein the terms “stabilized” and “locked” are used interchangeably, for example in relation to the effect of cross-linking in stabilizing or locking the RSV F protein in its pre-fusion conformation. These terms do not require 100% stability. Rather these terms denote a degree of improved or increased stability. For example, in some embodiments, when the term “stabilized” is used in relation to a RSV F protein cross-linked in its pre-fusion conformation, the term denotes that the pre-fusion conformation has greater stability than it would have had prior to or without such cross-linking. Stability, and relative stability, may be measured in various ways as described in other sections of this application, for example based on the half-life of the RSV pre-fusion conformation. The improvement or increase in stability may be to any degree that is useful or significant for the intended application. For example, in some embodiments stability may be increased by about 10%, 25%, 50%, 100%, 200% (i.e. 2-fold), 300% (i.e. 3-fold), 400% (i.e. 4-fold), 500% (i.e. 5-fold), 1000% (i.e. 10-fold), or more.

### RSV F Polypeptides, Proteins & Protein Complexes

The RSV Fusion or “F” protein is the envelope glycoprotein of respiratory syncytial viruses. The RSV F protein may be translated as a single polypeptide precursor in either a soluble (without the transmembrane domain) or membrane-bound (with the transmembrane domain) form. This polypeptide forms a trimer, which may, in some situations, be proteolytically cleaved by one or more cellular proteases at conserved furin consensus cleavage sites to yield two disulfide-bonded fragments known as the F1 (C-terminal) and F2 (N-terminal) fragments. The F2 fragment includes approximately the first 83 amino acids of the RSV F precursor. Either the uncleaved precursor protein, or a heterodimer of the cleaved F2 and F1 fragments, can form an RSV F protomer. Three such protomers assemble to form the final RSV F protein complex, which is a homotrimer of three protomers.

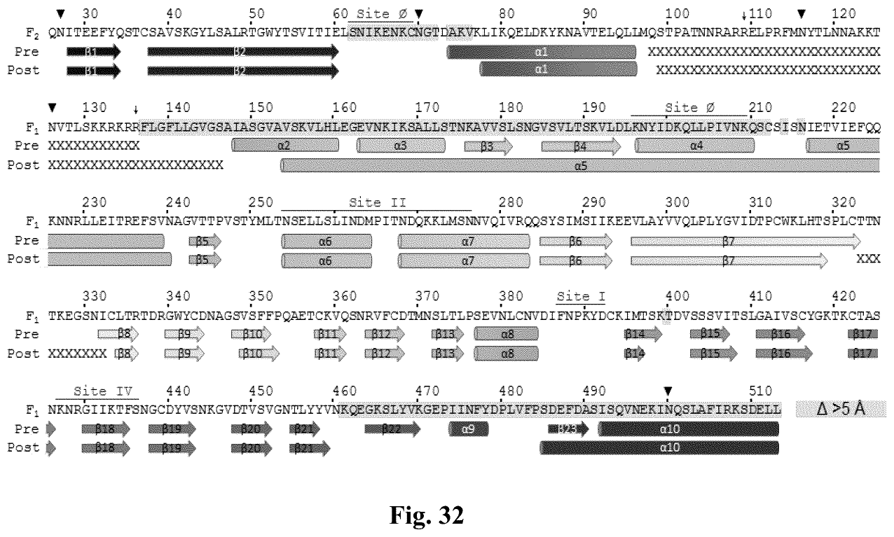

The RSV F protein trimer mediates fusion of viral and cellular membranes. The pre-fusion conformation of the RSV F protein trimer (which may be referred to herein as “pre-F”) is highly unstable (metastable). However, once the RSV virus docks with the cell membrane, the RSV F protein trimer undergoes a series of conformational changes and transitions to a highly stable post-fusion (“post-F”) conformation. The RSV F protein is known to induce potent neutralizing antibodies (nAbs) that correlate with RSV protection. For example, immunization with the RSV F protein induces nAbs that are protective in humans (e.g. Synagis). Several neutralizing epitopes (sites I, II and IV) are present on the post-fusion form of RSV F protein. Recently, however, Magro et al. showed that incubation of human sera with the RSV F protein in its post-fusion conformation failed to deplete the majority of neutralizing activity against the F protein, indicating the presence of neutralizing antigenic sites unique to the pre-fusion conformation (Magro et al. 2012, PNAS 109 (8): 3089). By x-ray crystallography, the epitopes recognized by palivizumab (Synagis), motavizumab (Numax), and that of the more recently discovered 101F monoclonal antibody (Mclellan et al., 2010, J. Virol., 84 (23): 12236-441; and Mclellan et al., 2010, Nat. Struct. Mol. Biol., February 17 (2): 248-50) were mapped. Most recently, Mclellan et al. (Science 340:1113-1117 (2013)) solved the structure of the F protein in its pre-fusion conformation, which revealed a novel neutralizing epitope—site ø—that is only displayed in the pre-fusion conformation, and to which a series of antibodies bind, e.g. 5C4, that are up to 50-fold more potently neutralizing than Synagis and Numax. Accordingly, there is mounting evidence that an RSV vaccine immunogen in this pre-fusion conformation and displaying site ø could elicit effective protection. However, to date the highly unstable (metastable) nature of the pre-fusion conformation of the RSV F protein has proved to be a significant barrier to the development of such a vaccine. Based on a comparison of the pre- and post-fusion RSV F structures of Mclellan et al. there appear to be two regions of the F protein that undergo large conformational changes (>5 Å). These regions are located at the N- and C-termini of the F1 subunit (residues 137-216 and 461-513, respectively) (see FIG. 32). In the crystal structure of the RSV F protein held in its pre-fusion conformation by the D25-antibody bound to the site ø epitope, the C-terminal F1 residues can be stabilized in the pre-fusion conformation by appending a foldon trimerization domain. To stabilize the N-terminal region of F1, Mclellan et al. found that binding of the antibody D25 was sufficient for crystallographic studies. However, for production of a vaccine immunogen alternative stabilization strategies are needed, such as those that do not require the RSV F protein to be bound to a large antibody molecule. One alternative approach that has been attempted involved the introduction of paired cysteine mutations (for disulfide bond formation) and cavity-filling mutations near the F1 N-terminus (see the DS-Cav1 RSV F protein variant described in Mclellan et al. (2013) Science 342:592-598, which is hereby incorporated by reference in its entirety). However, crystallographic analysis of such variants revealed that the structure was only partially in the pre-fusion conformation. Accordingly, additional engineering of the RSV F protein is needed in order to achieve an immunogen for clinical vaccine development.

The present invention provides certain alternative approaches for stabilizing the RSV F protein in its pre-fusion conformation, including providing specific locations within the RSV F protein that can be or should be cross-linked, and providing mutant forms of the RSV F protein that can facilitate the formation of such cross-links. Such cross-links and mutations can be used alone (e.g. in the context of a wild type RSV F protein or in the context of a RSV F protein that does not comprise any man made mutations or other man made modifications), or can be used in combination with one or more other man made mutations, modifications, cross-links, or stabilization strategies. Thus, for example, the approaches described herein can be used in conjunction with the use of added foldon trimerization domains, stabilizing antibodies (such as D25), and/or other partially or potentially stabilizing modifications or mutations-such as those in the DS-Cav1 RSV F protein variant described by Mclellan et al.

The present inventors have performed extensive analysis of the structure of the RSV F protein and have developed a variety of novel design strategies and novel engineered RSV F polypeptides, proteins and protein complexes. The present invention also provides methods for making and using such RSV F polypeptides, proteins, and protein complexes. In some embodiments, the present invention provides specific locations within the amino acid sequence of the RSV F protein at which, or between which, targeted cross-links can be made in order to “lock” the RSV F protein in its pre-F conformation. In some embodiments, the targeted cross-links are di-tyrosine cross-links. Where di-tyrosine cross-links are used, the present invention provides specific amino acid residues (or pairs of amino acid residues) that either comprise a pre-existing tyrosine residue or can be or are mutated to a tyrosine residue such that di-tyrosine cross-links can be made.

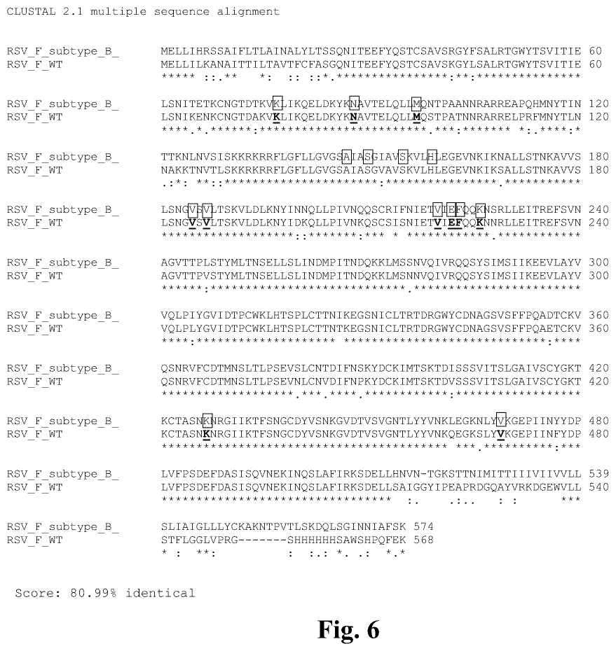

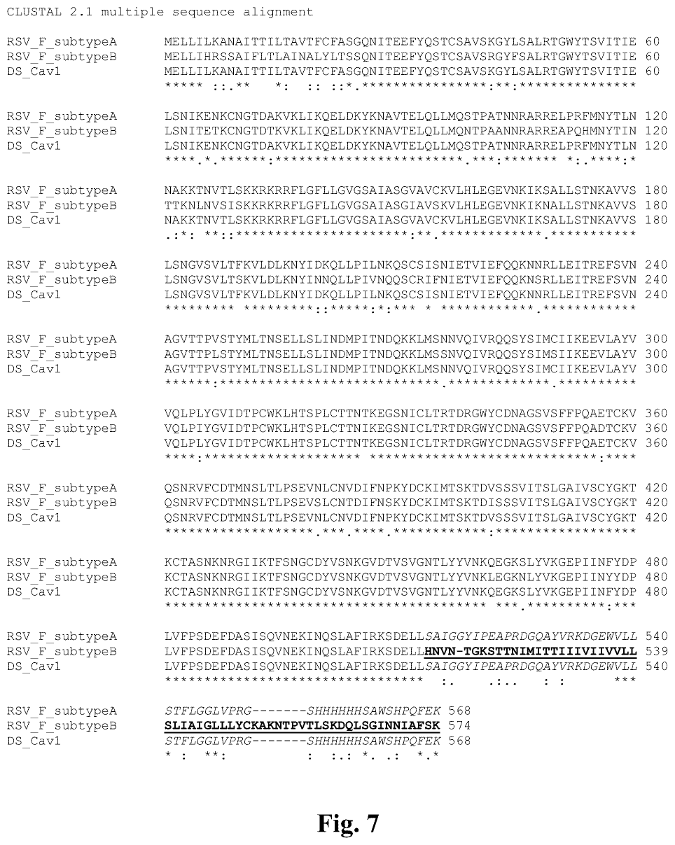

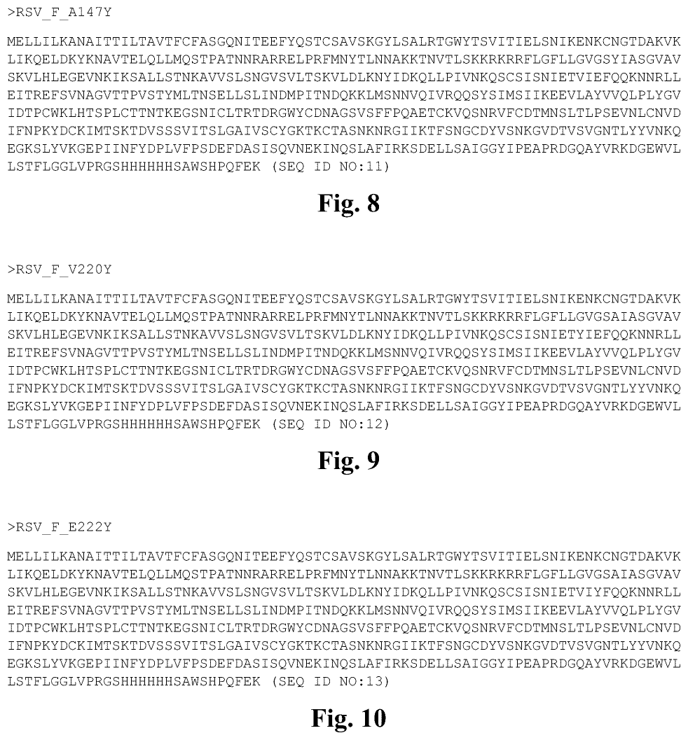

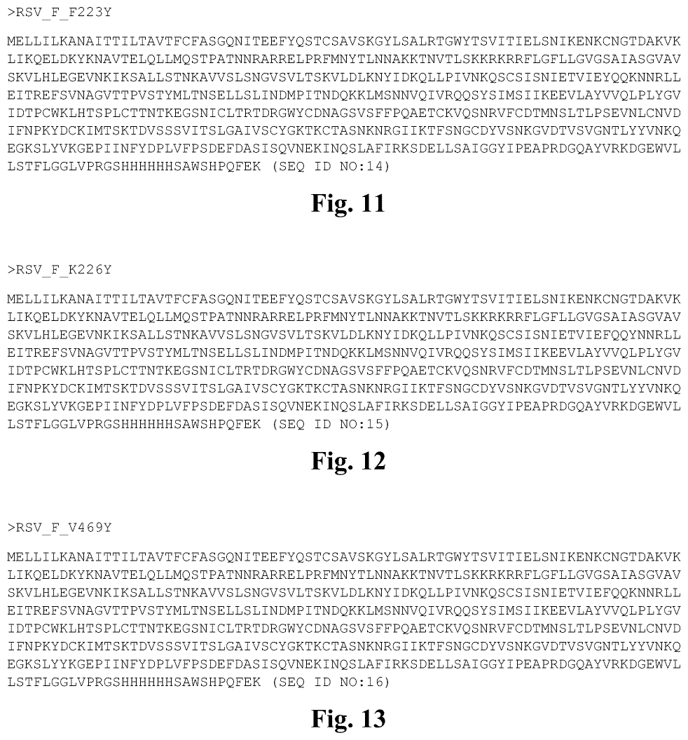

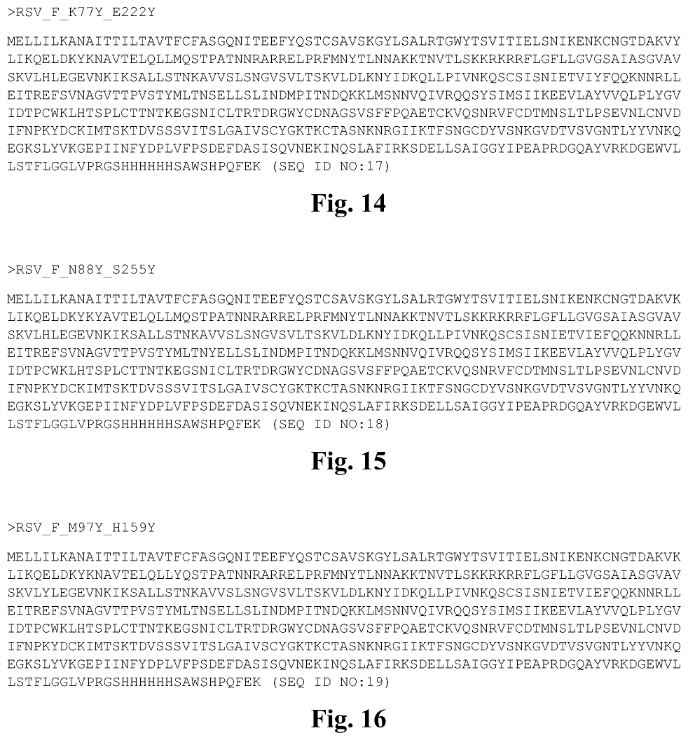

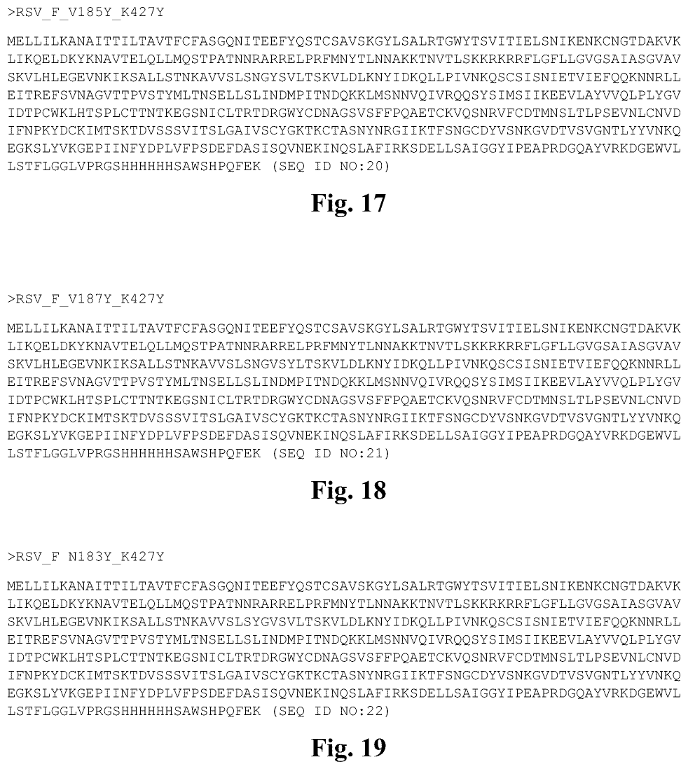

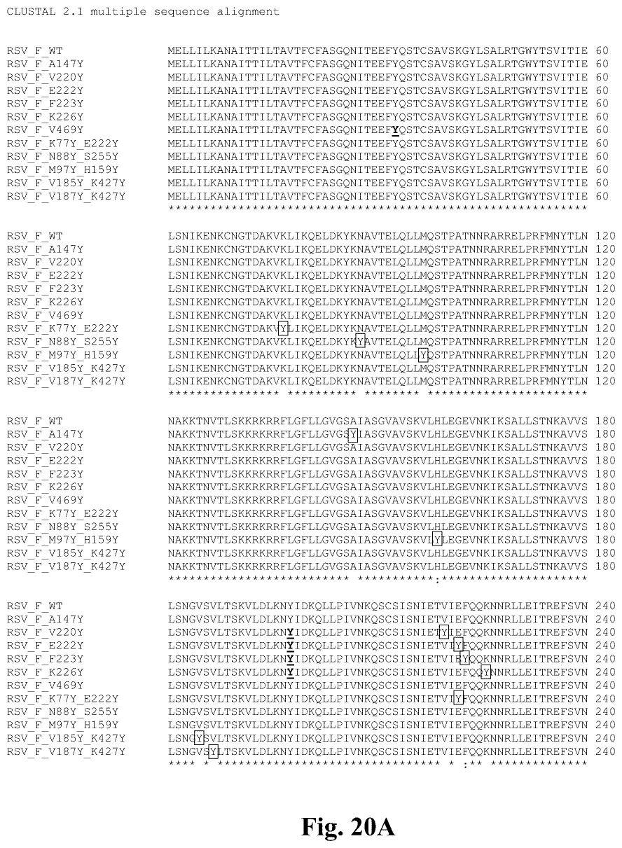

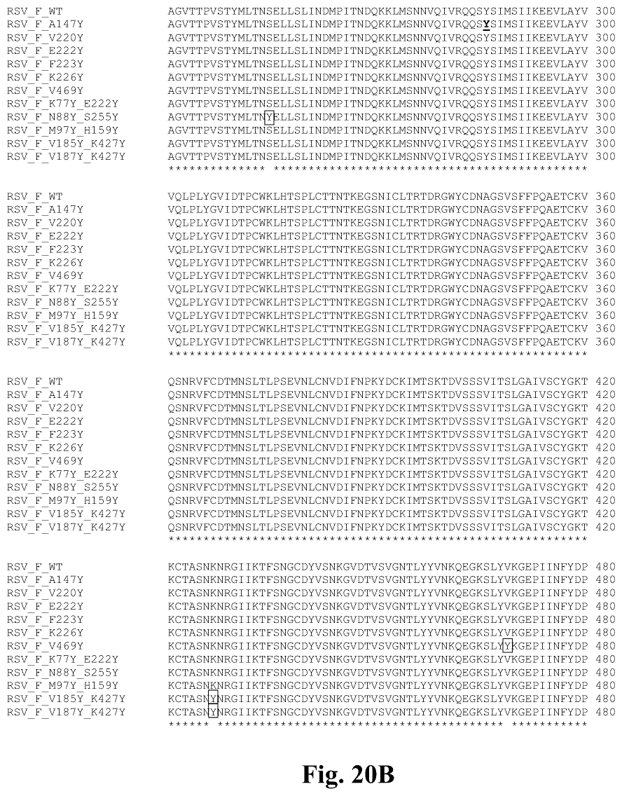

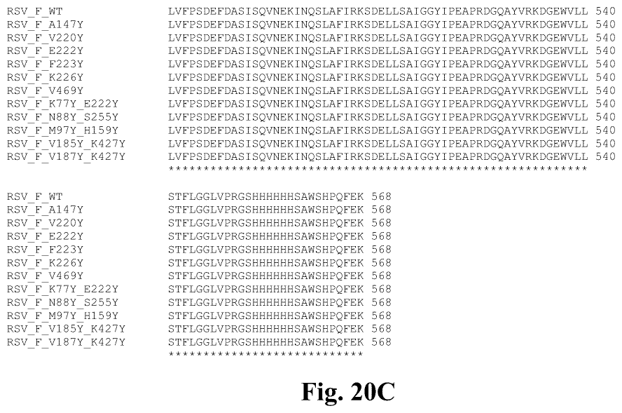

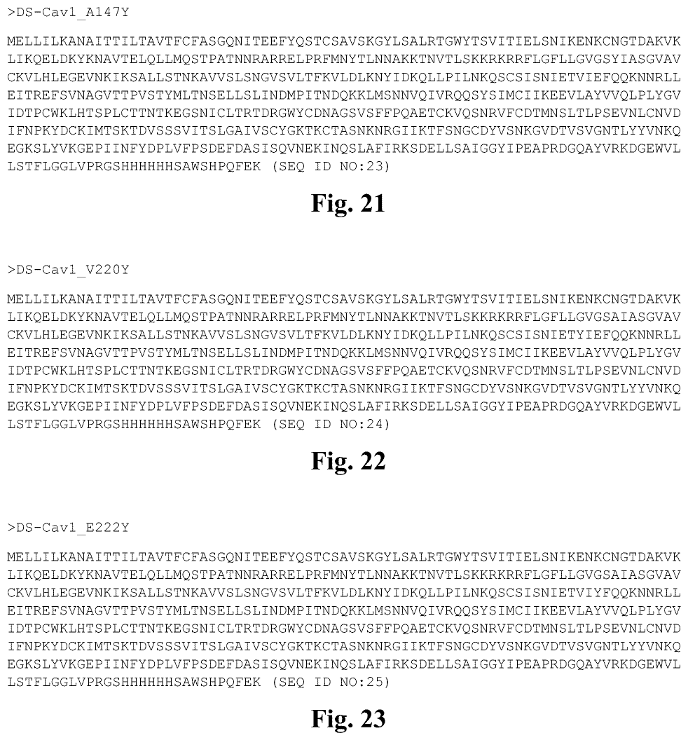

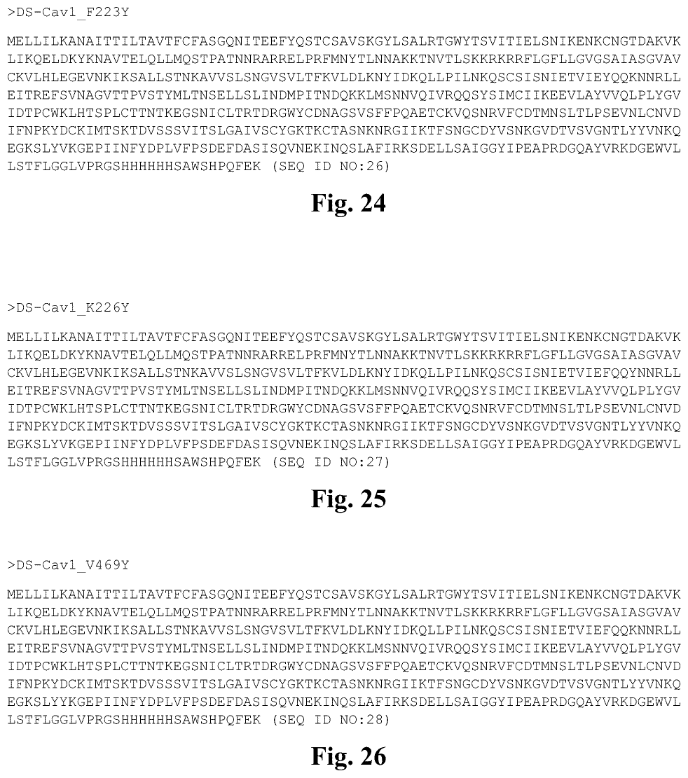

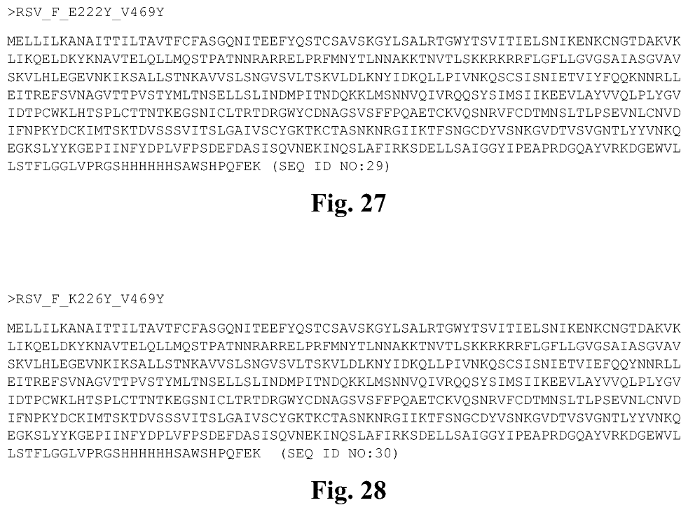

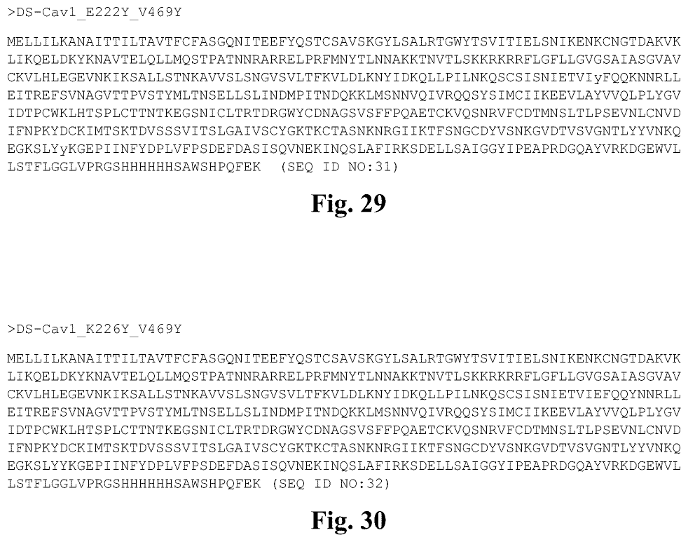

Throughout the present patent specification, when reference is made to specific amino acid residues or specific amino acid regions in the RSV F protein by referring to their amino residue number or numbers (such as amino acid residues 77, 88, 97, or 222, for example), and unless otherwise stated, the numbering is based on the RSV amino acid sequences provided herein in the sequence listing and in the Figures (see, for example, FIG. 6 and SEQ ID NO: 1). However, it should be noted, and one of skill in the art will understand, that different RSV sequences may have different numbering systems, for example, if there are additional amino acid residues added or removed as compared to SEQ ID NO: 1. As such, it is to be understood that when specific amino acid residues are referred to by their number, the description is not limited to only amino acids located at precisely that numbered position when counting from the beginning of a given amino acid sequence, but rather that the equivalent/corresponding amino acid residue in any and all RSV F sequences is intended-even if that residue is not at the same precise numbered position, for example if the RSV sequence is shorter or longer than SEQ ID NO. 1, or has insertions or deletions as compared to SEQ ID NO. 1. One of skill in the art can readily determine what is the corresponding/equivalent amino acid position to any of the specific numbered residues recited herein, for example by aligning a given RSV F sequence to SEQ ID NO. 1 or to any of the other RSV F amino acid sequences provided herein.

The present invention provides RSV F protein and polypeptide amino acid sequences, and compositions and methods comprising such sequences. However, the invention is not limited to the specific RSV F sequences disclosed herein. Rather the present invention contemplates variations, modifications and derivatives of the specific sequences provided herein.

In some embodiments, the RSV F polypeptides, proteins or protein complexes of the present invention can be derived from (or can comprise, consist essentially of, or consist of) the amino acid sequences of any suitable RSV F polypeptide, protein or protein complex sequence known in the art, including, without limitation: the amino acid sequence of RSV subtype A (for example in soluble form (SEQ ID NO:1, or amino acid residues 1-513 thereof) or in a full-length form (SEQ ID NO:2)); the amino acid sequence of RSV subtype B (for example in soluble form (SEQ ID NO:3, or amino acid residues 1-513 thereof) or in full-length form (SEQ ID NO:4)); the amino acid sequence of RSV variant DS-Cav1 (for example in soluble form (SEQ ID NO:5, or amino acid residues 1-513 thereof) or in full-length form (SEQ ID NO:6)); the amino acid sequence of RSV variant Cav1 (for example in soluble form (SEQ ID NO:7, or amino acid residues 1-513 thereof) or in full-length form (SEQ ID NO:8)); or the amino acid sequence of RSV variant DS (for example in soluble form (SEQ ID NO:9, or amino acid residues 1-513 thereof) or in full-length form (SEQ ID NO:10)), or any fragment thereof. In some embodiments, the RSV F proteins and polypeptides of the present invention can be derived from (or can comprise, consist essentially of, or consist of) amino acid sequences that have at least about 60%, 65%, 70%, 75%, 80%, 85%, 90%, 95%, 98%, or 99% sequence identity to any known RSV F sequences or RSV F ectodomain sequences (including but not limited to amino acid residues 1-513 of SEQ ID NOs: 1-10, or SEQ ID NOs: 1-10), or to RSV F sequences from any known RSV groups, subgroups, families, subfamilies, types, subtypes, genera, species, strains, and/or clades, or any fragment thereof. It should be noted that amino acid residues 1-513 of the various RSV F sequences provided herein are core RSV F ectodomain sequences. Amino acid residues 514 onwards in all such sequences comprise additional domains that may be present in some embodiments but not in others. In some embodiments variants of such additional domains may be present. For example, amino acid residues 514 onwards in all soluble RSV F sequences provided herein comprise an optional foldon trimerization domain, thrombin cleavage site, 6×His-tag (SEQ ID NO: 46), and a strep tag. In some embodiments these additional sequences may be absent, modified, rearranged or replaced. For example, in some embodiments different trimerization domains may be used, or different epitope tags may be used. Similarly, amino acid residues 514 onwards in all “full-length” or membrane-bound RSV F sequences provided herein comprise an optional RSV F protein transmembrane region and cytoplasmic tail. In some embodiments these additional sequences may be absent, modified, rearranged or replaced, for example with different transmembrane or cytoplasmic domains.

In some embodiments the present invention provides RSV F polypeptides, proteins, and/or protein complexes that comprise one or more artificially-introduced cross-links, wherein at least one of the following amino acid residues within the RSV F polypeptides, proteins, and/or protein complexes is artificially cross-linked to another amino acid residue in the RSV F protein: Y33, K77, N88, M97, A147, S150, S155, H159, N183, V185, V187, Y198, V220, E222, F223, K226, S255, Y286, K427 and V469. In some such embodiments the cross-link is a di-tyrosine cross-link.

In some embodiments the present invention provides RSV F polypeptides, proteins, and/or protein complexes that comprise one or more artificially-introduced cross-links, wherein such artificially introduced cross-links connect two of the following amino acid residues: Y33, K77, N88, M97, A147, S150, S155, H159, N183, V185, V187, Y198, V220, E222, F223, K226, S255, Y286, K427 and V469. In some such embodiment the cross-link is a di-tyrosine cross-link.

In some embodiments the present invention provides RSV F polypeptides, proteins, and/or protein complexes in which the amino acid residues in one or more of the following pairs of amino residues are cross-linked to each other by an artificially introduced cross-link: 147/286, 198/220, 198/222, 198/223, 198/226, 33/496, 77/222, 88/255, 97/159, 183/427, 185/427, and 187/427. In some such embodiments the cross-link is a di-tyrosine cross-link.

In some embodiments the present invention provides RSV F polypeptides, proteins, and/or protein complexes comprising an artificially introduced cross-link between two of the following regions: the F1 mobile N-terminus (residues 137-216), α2 (residues 148-160), α3 (residues 163-173), β3 (residues 176-182), β4 (residues 186-195), α4 (residues 197-211), the F1 mobile C-terminus (residues 461-513), β22 (residues 464-471), α9 (residues 474-479), β23 (residues 486-491), and α10 (residues 493-514). In some such embodiments the cross-link is a di-tyrosine cross-link.

In some embodiments the present invention provides RSV F polypeptides, proteins, and/or protein complexes comprising an artificially introduced cross-link between two of the following regions: amino acid residues from about position 67 to about position 87, amino acid residues from about position 78 to about position 98, amino acid residues from about position 87 to about position 107, amino acid residues from about position 137 to about position 157, amino acid residues from about position 140 to about position 160, amino acid residues from about position 145 to about position 165, amino acid residues from about position 149 to about position 169, amino acid residues from about position 173 to about position 193, amino acid residues from about position 175 to about position 195, from about position 177 to about position 197, amino acid residues from about position 188 to about position 208, amino acid residues from about position 210 to about position 230, amino acid residues from about position 212 to about position 232, amino acid residues from about position 213 to about position 233, amino acid residues from about position 216 to about position 236, amino acid residues from about position 245 to about position 265, amino acid residues from about position 276 to about position 296, amino acid residues from about position 417 to about position 437, and amino acid residues from about position 459 to about position 479. In some such embodiments the cross-link is a di-tyrosine cross-link.

In embodiments where the RSV F polypeptides, proteins, and/or protein complexes of the invention comprise one or more di-tyrosine cross-links, di-tyrosine cross-links may be introduced between two endogenous tyrosine residues, between two tyrosine residues originating from “to-tyrosine” mutations, or between a tyrosine residue originating from a “to-tyrosine” mutation and an endogenous tyrosine residue. In some embodiments, more than one di-tyrosine cross-link is introduced into a RSV F protein or polypeptide.

In embodiments where the RSV F polypeptides, proteins, and/or protein complexes of the invention comprise one or more di-tyrosine cross-links, non-limiting examples of amino acid positions where a “to-tyrosine” mutation can be introduced include K77, N88, M97, A147, S150, S155, H159, N183, V185, V187, V220, E222, F223, K226, S255, K427 and V469 (see FIG. 6), or any combination thereof.

In embodiments where the RSV F polypeptides, proteins, and/or protein complexes of the invention comprise one or more di-tyrosine cross-links, non-limiting examples of preexisting or endogenous tyrosine residues that can be used to form a di-tyrosine cross-link include Y33, Y198 and Y286 (see FIG. 6), or any combination thereof.

In embodiments where the RSV F polypeptides, proteins, and/or protein complexes of the invention comprise one or more di-tyrosine cross-links, non-limiting examples of residue pairs between which a di-tyrosine cross-link can be introduced include 147/286, 198/220, 198/222, 198/223, 198/226, 33/496, 77/222, 88/255, 97/159, 183/427, 185/427, and 187/427, or any combination thereof.

In embodiments where the RSV F polypeptides, proteins, and/or protein complexes of the invention comprise one or more di-tyrosine cross-links, non-limiting examples of regions or secondary structures of the RSV F protein from which amino acids may be selected for tyrosine substitution and/or di-tyrosine cross-linking include the F1 mobile N-terminus (residues 137-216), α2 (residues 148-160), α3 (residues 163-173), β3 (residues 176-182), β4 (residues 186-195), α4 (residues 197-211), the F1 mobile C-terminus (residues 461-513), β22 (residues 464-471), α9 (residues 474-479), β23 (residues 486-491), and α10 (residues 493-514). Non-limiting examples of other regions of RSV F protein from which one or more amino acids may be selected for tyrosine substitution and/or cross-linking include residues from about position 67 to about position 87, from about position 78 to about position 98, from about position 87 to about position 107, from about position 137 to about position 157, from about position 140 to about position 160, from about position 145 to about position 165, from about position 149 to about position 169, from about position 173 to about position 193, from about position 175 to about position 195, from about position 177 to about position 197, from about position 188 to about position 208, from about position 210 to about position 230, from about position 212 to about position 232, from about position 213 to about position 233, from about position 216 to about position 236, from about position 245 to about position 265, from about position 276 to about position 296, from about position 417 to about position 437, and from about position 459 to about position 479.

In some embodiments, the present invention provides RSV F polypeptides, proteins, and/or protein complexes that are derived from, comprise, consist essentially of, or consist of, the amino acid sequence of SEQ ID NO:11, 12, 13, 14, 15, 16, 17, 18, 19, 20, 21, 22, 23, 24, 25, 26, 27, 28, 29, 30, 31, or 32, or amino acid residues 1-513 thereof, (each of which are mutants of the RSV F amino acid sequence that comprise one or more “to tyrosine” mutations to facilitate di-tyrosine cross-linking and to facilitate “locking” of the RSV F protein in its pre-F conformation), or any fragment thereof, such as fragments comprising amino acid residues 1-513 thereof, and/or fragments comprising the F1 or F2 fragments of the RSV F protein, or any other fragments of the RSV F protein that may be generated proteolytically and/or that may be assembled into or form part of a functional RSV F protein. In some embodiments, the present invention provides RSV F polypeptides, proteins, and/or protein complexes that are derived from, comprise, consist essentially of, or consist of, an amino acid sequence having at least about 75%, 80%, 85%, 90%, 95%, 98%, or 99% sequence identity to SEQ ID NO: 11, 12, 13, 14, 15, 16, 17, 18, 19, 20, 21, 22, 23, 24, 25, 26, 27, 28, 29, 30, 31, or 32, or amino acid residues 1-513 thereof, or any fragment thereof.

Non-limiting examples of amino acid positions in an RSV F protein or polypeptide to which di-tyrosine cross-links may be targeted include Y33 (pre-existing Tyr residue) and V469Y (to-Tyr substitution), where a F2-F1 intra-protomer bond will form, the positions Y198 (pre-existing Tyr residue) and E222Y (to-Tyr substitution), where an F1-F1 intra-molecular bond would form, the positions K77Y (to-Tyr substitution) and E222Y (to-Tyr substitution), where an F₂-F₁ inter-protomer bond would form, the positions N88Y (to-Tyr substitution) and S255Y (to-Tyr substitution), where an F₂-F₁ inter-protomer bond would form, the positions M97Y (to-Tyr substitution) and H159Y (to-Tyr substitution), where an F₂-F₁ inter-protomer bond would form, the positions V185Y (to-Tyr substitution) and K427Y (to-Tyr substitution), where an F₁-F₁ inter-protomer bond would form, and the positions N183Y (to-Tyr substitution) and K427Y (to-Tyr substitution), where an F₁-F₁ inter-protomer bond would form. These positions were initially identified by analysis of the atomic level structure of the RSV prefusion F protein. Further non-limiting examples include positions A147Y (to-Tyr substitution) and Y286 (pre-existing Tyr), Y198 (pre-existing Tyr residue) and V220Y (to-Tyr substitution), Y198 (pre-existing Tyr residue) and F223Y (to-Tyr substitution), Y198 (pre-existing Tyr residue) and K226Y (to-Tyr substitution), V187Y (to-Tyr substitution) and K427Y (to-Tyr substitution) and N88Y (to-Tyr substitution) and S255Y (to-Tyr substitution). In some embodiments, the RSV polypeptides, proteins or protein complexes of the invention comprise one of the above listed di-tyrosine cross-links. In some embodiments, the RSV polypeptides, proteins or protein complexes of the invention comprise two of the above listed di-tyrosine cross-links. In some embodiments, the RSV polypeptides, proteins or protein complexes of the invention comprise three of the above listed di-tyrosine cross-links. In some embodiments, the RSV polypeptides, proteins or protein complexes of the invention comprise four of the above listed di-tyrosine cross-links. In some embodiments, the RSV polypeptides, proteins or protein complexes of the invention comprise five or more of the above listed di-tyrosine cross-links. In some embodiments, the RSV polypeptides, proteins or protein complexes of the invention comprise any combination or one or more of the above listed di-tyrosine cross-links.

Non-limiting examples of RSV F proteins designed to have more than one di-tyrosine cross-link include RSV F proteins with two “to-tyrosine” mutations (E222Y/V469Y), for example, derived from subtype A (SEQ ID NO:29) or DS-Cav1 (SEQ ID NO:31) where the tyrosine substituted at position 222 is designed to pair with the endogenous tyrosine at position 198, and the tyrosine substituted at position 469 is designed to pair with the endogenous tyrosine at position 33, thus stabilizing the RSV F protein by the formation of two di-tyrosine cross-links; and RSV F proteins with two to-tyrosine mutations (K226Y/V469Y), for example, derived from subtype A (SEQ ID NO:30) or DS-Cav1 (SEQ ID NO:32) where the tyrosine substituted at position 226 is designed to pair with the endogenous tyrosine at position 198, and the tyrosine substituted at position 469 is designed to pair with the endogenous tyrosine at position 33, thus stabilizing the F protein by the formation of two di-tyrosine cross-links.

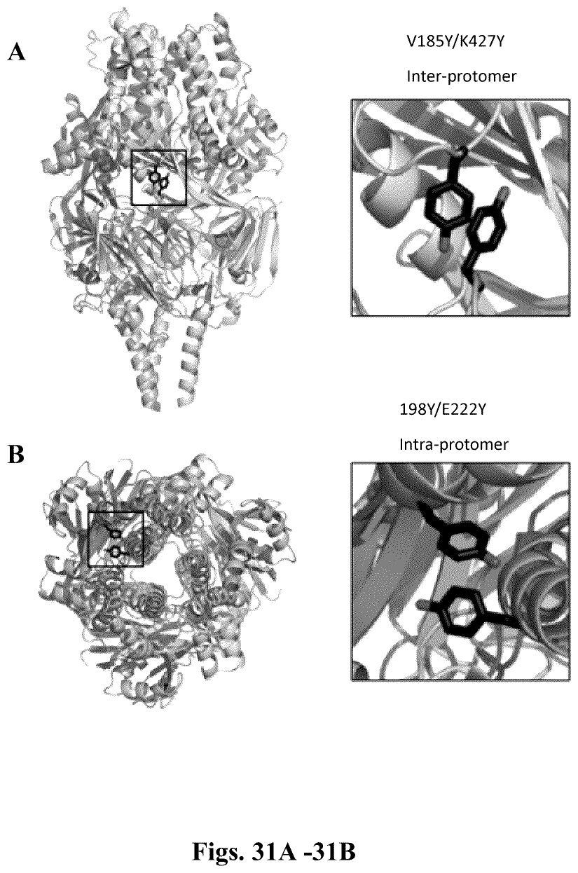

As described above, each protomer of the mature RSV F trimer may be cleaved into two distinct polypeptide chains termed F1 and F2 which associate non-covalently to form a protomer. A bond between a F1 polypeptide and a F2 polypeptide within the same protomer is an example of an inter-molecular bond and an intra-protomer bond. The invention provides exemplary RSV F proteins and polypeptides comprising cross-links designed to stabilize this interaction, including without limitation, SEQ ID NO:16 (V469Y, where the introduced tyrosine at position 469 is designed to pair with endogenous tyrosine 33), SEQ ID NO:17 (K77Y/E222Y, designed to form a di-tyrosine pair between the introduced tyrosines), SEQ ID NO:18 (N88Y/S255Y, designed to form a di-tyrosine pair between the introduced tyrosines), and SEQ ID NO:19 (M97Y/H159Y, designed to form a di-tyrosine pair between the introduced tyrosines), as well as RSV F polypeptides, proteins or protein complexes derived from such sequences and including the specific “to tyrosine” mutations present in such sequences. The invention also provides exemplary RSV F proteins and polypeptides comprising cross-links designed to hold two protomers of the trimer together (inter-molecular, inter-protomer bond), including without limitation, SEQ ID NO:20 (V185Y/K427Y), SEQ ID NO:21 (V187Y/K427Y) SEQ ID NO:22 (N183Y/K427Y), as well as RSV F polypeptides, proteins or protein complexes derived from such sequences and including the specific “to tyrosine” mutations present in such sequences. In each of these proteins, one introduced tyrosine in one protomer is designed to pair with the other introduced tyrosine on the adjacent protomer. For example, in SEQ ID NO:20 (V185Y/K427Y), the tyrosine at position 185 on “protomer A” would form a di-tyrosine bond with the tyrosine at position 427 on “protomer B” (see FIG. 31A).

In some embodiments, the F1 polypeptide of a RSV F protein is cross-linked with the F2 polypeptide of the same protomer (inter-molecular/intra-protomer bond). In some embodiments, the F1 polypeptide is intra-molecularly cross-linked (e.g., both tyrosines of the cross-link are located within the same F1 polypeptide). In some embodiments, the F2 polypeptide is intra-molecularly cross-linked (e.g., both tyrosines of the cross-link are located within the same F1 polypeptide). In some embodiments, the F1 polypeptide of the RSV prefusion F protein is cross-linked with the F1 polypeptide of an adjacent protomer (inter-protomer bond). In some embodiments, the F1 polypeptide of the RSV prefusion F protein is cross-linked with the F2 polypeptide of an adjacent protomer (inter-protomer bond).

The transition from the pre-F to the post-F structures involves very significant rearrangement of parts of the RSV protein, in particular the C- and N-termini of F₁, while the rest of the protein moves significantly less. In order to stabilize the preF conformation by the methods of this invention, parts of the protein that move significantly (e.g. more than 5 Å) can be attached to parts of the protein that move less significantly (e.g. less than 5 Å), either between two residues of the F₁ chain of a single protomer, between two residues of the F₂ chain of single protomer, between one residue of the F₁ chain and one residue of the F₂ chain within the same protomer, or between F₁ and/or F₂ residues of two adjacent protomers. Alternatively, parts of the protein that move significantly (e.g. more than 5 Å) can be attached to other parts of the protein that also move significantly (e.g. more than 5 Å), also either between two residues of the F₁ chain, between two residues of the F₂ chain, between one residue of the F₁ chain and one residue of the F₂ chain of within the same protomer, or between F₁ and/or F₂ residues of two adjacent protomers. Covalent attachment of moving parts to either moving or non-moving parts prevents the transition from the prefusion structure to either intermediate structures or to the postfusion structure.

Positions in F₂ that move more than 5 Å in the pre-fusion to post-fusion transition include the positions 62 through 76, whereas positions 26 through 61 and 77 through 97 move less than 5 Å (and positions 98-109 have yet to be determined in the pre-fusion structure). Positions in F₁ that move more than 5 Å in the pre-F to post-F transition include the positions 137 through 216 (F₁ mobile N-terminus) and 461 through 513 (F₁ mobile C-terminus). The F₁ mobile N-terminus of the preF structure further comprises the 2 (positions 148 through 173), 3 (positions 176 through 182), and 4 (positions 197 through 211) secondary structures that can each either be attached to one another, or to other moving or non-moving parts within the same protomer or between protomers of the F protein trimer (complex consisting of three protomers). The F₁ mobile C-terminus of the preF structure further comprises the 22 (positions 464 479), 3 (positions 486 t structures that can each either be attached to one another, or to other moving or non-moving parts within the same protomer or between protomers of the F protein trimer. (See FIG. 32.)

In some embodiments (including all of those described above, and those involving RSV F polypeptides, proteins, and/or protein complexes having any of the specific amino acid sequences recited herein, and those involving variants or fragments of such RSV F polypeptides, proteins, and/or protein complexes having less than 100% identity to the specific amino acid sequence provided herein), the RSV F polypeptides, proteins, and/or protein complexes of the invention should have one or more desired properties such as being capable of (1) forming the pre-F conformation, (2) being “locked” in the pre-F conformation by cross-linking, (3) binding to a pre-F specific antibody, (4) binding to an antibody that binds to site ø, (5) binding to a neutralizing antibody, (6) binding to a broadly neutralizing antibody, (7) binding to an antibody selected from the group consisting of D25, AM22, 5C4, 101F (8) binding to palivizumab (Synagis), (9) binding to and/or activating a B cell receptor, (10) eliciting an antibody response in an animal, (11) eliciting a protective antibody response in an animal, (12) eliciting production of neutralizing antibodies in an animal, (13) eliciting production of broadly neutralizing antibodies in an animal, (14) eliciting production of antibodies that recognize quaternary neutralizing epitopes (QNEs) in an animal, and/or (15) eliciting a protective immune response in an animal.

Unless otherwise stated, all description herein that relates to specific RSV F polypeptides, proteins, and protein complexes, relates equally to all homologs, orthologs, analogs, derivatives, mutant forms, fragments, chimeras, fusion proteins etc. thereof, such as those that have certain desired properties or features (for example those that are in the pre-F conformation, or that are capable of forming part of a complex having the desired pre-F conformation, or that have desired functional properties, including, but not limited to, being capable of binding to, or eliciting the production of, one or more anti-RSV antibodies, such as antibodies that are specific to the RSV pre-F conformation and/or that bind to the ø site).

Similarly, all description herein that relates to specific polypeptides, proteins, and/or protein complexes polypeptides, proteins, and/or protein complexes (e.g. those having specific amino acid sequences or those from a specific RSV type, subtype, or strain) relates equally to other related forms of such polypeptides, proteins, and/or protein complexes that may exist in nature (for example in different RSV types, subtypes or strains) or that are related to the specific sequences provides herein but have been altered artificially in some way, such as by recombinant means, chemical means, or any other means. The polypeptides, proteins, and/or protein complexes described herein can have, or can be derived from, the nucleotide and/or amino acid sequences of any suitable RSV polypeptides, proteins, and/or protein complexes known in the art. In some embodiments, the RSV F polypeptides, proteins, and/or protein complexes of the invention may be, or may be derived from, derivatives and/or analogs of specific specific RSV F polypeptides, proteins, and/or protein complexes described herein or known in the art, including proteins that are substantially homologous to any such proteins, or fragments thereof (e.g., in various embodiments, those having at least about 40% or 50% or 60% or 70% or 75% or 80% or 85% or 90% or 95% or 98% or 99% identity with an amino acid or nucleic acid sequence of any specific RSV F polypeptides, proteins, and/or protein complexes described herein or known in the art, when aligned using any suitable method known to one of ordinary skill in the art, such as, for example, using a computer homology program known in the art) or whose encoding nucleic acid is capable of hybridizing to a coding nucleic acid sequence of a protein of the invention, under high stringency, moderate stringency, or low stringency conditions.

In some embodiments, the invention provides fragments of the specific RSV F polypeptides, proteins, and protein complexes described herein, such as those comprising, consisting essentially of, or consisting of, at least about 10 amino acids, 20 amino acids, 50 amino acids, 100 amino acids, 200 amino acids, or 500 amino acids.

In some embodiments one or more amino acid residues within a specific RSV F polypeptide, protein, and/or protein complex as described herein, or as known in the art, can be deleted, added, or substituted with another amino acid. In embodiments where such mutations are introduced, the RSV F polypeptides, proteins, or protein complexes can be micro-sequenced to determine a partial amino acid sequence. In other embodiments the nucleic acid molecules encoding the RSV F polypeptides, proteins, and/or protein complexes can be sequenced to identify and/or confirm the introduction of mutations.

In some embodiments, one or more amino acid residues can be substituted by another amino acid having a similar polarity and that may acts as a functional equivalent, resulting in a silent alteration. In some embodiments substitutions for an amino acid within the sequence may be selected from other members of the class to which the amino acid belongs e.g. to create a conservative substitution. For example, the nonpolar (hydrophobic) amino acids include alanine, leucine, isoleucine, valine, proline, phenylalanine, tryptophane and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Such substitutions are generally understood to be conservative substitutions.

In some embodiments artificial, synthetic, or non-classical amino acids or chemical amino acid analogs can be used to make the RSV F polypeptides, proteins, and/or protein complexes of the invention. Non-classical amino acids include, but are not limited to, the D-isomers of the common amino acids, fluoro-amino acids, and “designer” amino acids such as β-methyl amino acids, Cγ-methyl amino acids, Nγ-methyl amino acids, and amino acid analogs in general. Additional non-limiting examples of non-classical amino acids include, but are not limited to: α-aminocaprylic acid, Acpa; (S)-2-aminoethyl-L-cysteine/HCl, Aecys; aminophenylacetate, Afa; 6-amino hexanoic acid, Ahx; γ-amino isobutyric acid and α-aminoisobytyric acid, Aiba; alloisoleucine, Aile; L-allylglycine, Alg; 2-amino butyric acid, 4-aminobutyric acid, and α-aminobutyric acid, Aba; p-aminophenylalanine, Aphe; b-alanine, Bal; p-bromophenylalaine, Brphe; cyclohexylalanine, Cha; citrulline, Cit; β-chloroalanine, Clala; cycloleucine, Cle; p-cholorphenylalanine, Clphe; cysteic acid, Cya; 2,4-diaminobutyric acid, Dab; 3-amino propionic acid and 2,3-diaminopropionic acid, Dap; 3,4-dehydroproline, Dhp; 3,4-dihydroxylphenylalanine, Dhphe; p-flurophenylalanine, Fphe; D-glucoseaminic acid, Gaa; homoarginine, Hag; 8-hydroxylysine/HCl, Hlys; DL-β-hydroxynorvaline, Hnvl; homoglutamine, Hog; homophenylalanine, Hoph; homoserine, Hos; hydroxyproline, Hpr; p-iodophenylalanine, Iphe; isoserine, Ise; α-methylleucine, Mle; DL-methionine-S-methylsulfoniumchloide, Msmet; 3-(1-naphthyl) alanine, 1 Nala; 3-(2-naphthyl) alanine, 2Nala; norleucine, Nle; N-methylalanine, Nmala; Norvaline, Nva; O-benzylserine, Obser; O-benzyltyrosine, Obtyr; O-ethyltyrosine, Oetyr; O-methylserine, Omser; O-methylthreonine, Omthr; O-methyltyrosine, Omtyr; Ornithine, Orn; phenylglycine; penicillamine, Pen; pyroglutamic acid, Pga; pipecolic acid, Pip; sarcosine, Sar; t-butylglycine; t-butylalanine; 3,3,3-trifluroalanine, Tfa; 6-hydroxydopa, Thphe; L-vinylglycine, Vig; (−)-(2R)-2-amino-3-(2-aminoethylsulfonyl) propanoic acid dihydroxochloride, Aaspa; (2S)-2-amino-9-hydroxy-4,7-dioxanonanoic acid, Ahdna; (2S)-2-amino-6-hydroxy-4-oxahexanoic acid, Ahoha; (−)-(2R)-2-amino-3-(2-hydroxyethylsulfonyl) propanoic acid, Ahsopa; (−)-(2R)-2-amino-3-(2-hydroxyethylsulfanyl) propanoic acid, Ahspa; (2S)-2-amino-12-hydroxy-4,7,10-trioxadodecanoic acid, Ahtda; (2S)-2,9-diamino-4,7-dioxanonanoic acid, Dadna; (2S)-2,12-diamino-4,7,10-trioxadodecanoic acid, Datda; (S)-5,5-difluoronorleucine, Dfnl; (S)-4,4-difluoronorvaline, Dfnv; (3R)-1-1-dioxo-[1,4]thiaziane-3-carboxylic acid, Dtca; (S)-4,4,5,5,6,6,6-heptafluoronorleucine, Hfnl; (S)-5,5,6,6,6-pentafluoronorleucine, Pfnl; (S)-4,4,5,5,5-pentafluoronorvaline, Pfnv; and (3R)-1,4-thiazinane-3-carboxylic acid, Tca. Furthermore, the amino acid can be D (dextrorotary) or L (levorotary). For a review of classical and non-classical amino acids, see Sandberg et al., 1998 (Sandberg et al., 1998. New chemical descriptors relevant for the design of biologically active peptides. A multivariate characterization of 87 amino acids. J Med Chem 41 (14): pp. 2481-91).

### Nucleic Acids

In addition to providing certain RSV F polypeptides, proteins, and/or protein complexes, as described herein, the present invention also provides nucleic acids encoding such RSV F polypeptides, proteins, and/or protein complexes, and compositions and vectors comprising such nucleic acids. Such nucleic acids can be obtained or made using any suitable method known in the art. For example, nucleic acid molecules encoding RSV F polypeptides, proteins, and/or protein complexes may be obtained from cloned DNA or made by chemical synthesis. In some embodiments the nucleic acids may be obtained by reverse transcribing RNA prepared by any of the methods known to one of ordinary skill in the art, such as random- or poly A-primed reverse transcription. Whatever the source, a nucleic acid molecule encoding a RSV F polypeptide, protein, and/or protein complex of the present invention can be cloned into any suitable vector, such as those to be used for propagation of the nucleic acid molecule or those to be used for expression of the nucleic acid molecule. The nucleic acid may be cleaved at specific sites using various restriction enzymes, if needed. In embodiments requiring expression, the nucleic acid can be operatively linked to a promoter suitable for directing expression in the desired cell type, such as a mammalian cell or an insect cell, and may be incorporated into any suitable expression vector, such as a mammalian or insect expression vector.

In some embodiments, the RSV F polypeptides, proteins or protein complexes of the present invention can derived from nucleic sequences that encode (or that comprise, consist essentially of, or consist of nucleotide sequences that encode) any suitable RSV F polypeptide, protein or protein complex sequence known in the art, or any fragment thereof, including, without limitation: a nucleotide sequence that encodes the wild-type (WT) full length F protein from RSV subtype A (for example, SEQ ID NO:33), or a nucleotide sequence that encodes the wild-type (WT) full length F protein from RSV subtype B (for example, SEQ ID NO:34), or variants of such sequences that have been codon optimized for expression in cells of any particular species of interest, or that contain any mutations of interest. For example, in some embodiments, the RSV F polypeptides, proteins or protein complexes of the present invention can derived from nucleotide sequences that encode the F protein of RSV F type A, but that have been codon optimized for expression in human (e.g. SEQ ID NO:35), hamster (e.g. SEQ ID NO: 36), insect (e.g. SEQ ID NO:37), or mouse cells (e.g. SEQ ID NO:38), or optimized for expression in any other cell type. Similarly, in some embodiments, the RSV F polypeptides, proteins or protein complexes of the present invention can derived from nucleotide sequences that encode the F protein of RSV F type B, but that have been codon optimized for expression in human (e.g. SEQ ID NO:39), hamster (e.g. SEQ ID NO:40), insect (e.g. SEQ ID NO:41), or mouse cells (e.g. SEQ ID NO:42), or optimized for expression in any other cell type. Similarly, in some embodiments, the RSV F polypeptides, proteins or protein complexes of the present invention can derived from nucleotide sequences that encode the F protein of RSV F type A or B, and which may or may not have been codon optimized for expression cells of any given species of interest, and which also comprise one or more other mutations of interest. For example, in some embodiments, the RSV F polypeptides, proteins or protein complexes of the present invention can be derived from nucleotide sequences that comprise DS-CAV1 (SEQ ID NO: 43), DS (SEQ ID NO: 44), and/or CAV1 (SEQ ID NO:45) mutant sequences. Although the three specific sequences provided in SEQ ID NO:s 43, 44, and 45 comprise the DS, CAV1, and DS-CAV1 mutations in the context of a human codon optimized full-length RSV type A sequence, such mutations, or indeed any other mutations of interest (including all of the “to-tyrosine” mutations of the invention), could equally be present in a backbone of any other suitable RSV type A or type B sequence, including, but not limited to, those sequences that have been optimized for expression in any species of interest, or that include any mutations of interest, or that include only certain portions of the RSV type A or B sequences, such as, for example, only nucleotides 1-1539 that encode only the RSV F ectodomain (without the transmembrane and/or cytoplasmic domains). One of skill in the art will recognize that there are a variety of nucleotide sequences that can encode the various RSV F polypeptides and proteins described herein, and all such nucleotide sequences are intended to fall within the scope of the present invention. For example, in some embodiments, the RSV F proteins and polypeptides of the present invention can be derived from nucleotide sequences that have at least about 60%, 65%, 70%, 75%, 80%, 85%, 90%, 95%, 98%, or 99% sequence identity to any known nucleotide sequences that encode an RSV F protein, including, but not limited to, any of those illustrated herein (including SEQ ID NOs: 33, 34, 35, 36, 37, 38, 39, 40, 41, 42, 43, 44, and 45), or that have at least about 60%, 65%, 70%, 75%, 80%, 85%, 90%, 95%, 98%, or 99% sequence identity to nucleotides 1-1539 thereof (which encode the ectodomain sequences), or that have at least about 60%, 65%, 70%, 75%, 80%, 85%, 90%, 95%, 98%, or 99% sequence identity to nucleotide sequences that encode RSV F proteins from any known RSV groups, subgroups, families, subfamilies, types, subtypes, genera, species, strains, and/or clades, or any fragment thereof.

Furthermore, one or skill in the art can readily visualize, or make, nucleic acid molecules that comprise any one or more of the specific “to-tyrosine” mutations described herein, for example, by locating the nucleotide codon that encodes the specific amino acid residue to be mutated, and mutating the nucleotides at that codon as necessary to result in a tyrosine-encoding codon.

### Cross-Linking

In some embodiments the RSV F polypeptides and/or proteins of the invention are assembled into protein complexes having a desired conformational structure, such as the pre-F conformation, and are cross-linked in order to stabilize that conformation. Details of particular regions of the RSV F protein that can be cross-linked, as well as particular RSV mutants designed to facilitate such cross-linking, are described in other sections of this application. In some embodiments the cross-links may be used to stabilize the tertiary and/or quaternary structures of the RSV prefusion F protein. In some embodiments, the cross-linking may be intra- and/or intermolecular cross-linking. In some embodiments, the cross-links that are used are targeted cross-links. In some embodiments, the cross-links that are used are stable under physiological conditions. In some embodiments, the cross-links that are used do not lead to aggregate formation of the RSV prefusion F protein, for example during expression and/or during storage (such as storage of compositions comprising high concentrations of the RSV prefusion F protein). In some embodiments the introduction of such cross-links may enhance the effectiveness of the RSV polypeptides, proteins and proteins of the invention as immunogens, such as vaccine immunogens. In some embodiments the introduction of such cross-links may stabilize epitopes (such as the @ epitope) within the RSV F protein such that the epitopes can be recognized by particular antibodies, elicit production of antibodies, and/or activate B cell receptors upon antibody binding.

In some embodiments targeted cross-linking can be used. A targeted cross-link is one that can be made to form at a particular position or positions within the RSV F protein or protein complex. Several strategies may be used to target cross-links to specific locations in an RSV F protein or polypeptide, such as the specific locations described herein. The present invention provides residue pairs within the RSV F protein that, when cross-linked, can or may stabilize a RSV F polypeptide, protein, or protein complex in its pre-F conformation, and/or in a conformation that is capable of binding to, or eliciting the production of, neutralizing antibodies, and/or that is capable of generating a neutralizing antibody response in an animal. A targeted cross-link may be introduced at one or more of the locations or positions specified herein by exploiting the physical and/or chemical properties of certain amino acid side chains, for example by making use of enzymatic reactions that recognize specific amino acid sequences or three-dimensional structures, or by incorporating non-natural amino acids that have the ability to form cross-links in a folded protein or protein complex.

Cross-links or modifications may be targeted to specific sites in the structure of the RSV F protein or polypeptide in order to achieve the desired outcome, e.g. stabilization or the pre-F conformation. The present invention contemplates the targeted introduction of one or more cross-links and/or other stabilizing modifications at any suitable position(s) in a RSV F protein or polypeptide, preferably where the cross-link or modification stabilizes the RSV F protein or polypeptide in a pre-fusion conformation, or provides enhanced stabilization of the pre-fusion conformation. The invention contemplates that any RSV F protein amino acid residue, residue pair, secondary structure or other region described herein for di-tyrosine cross-linking may also be used in the formation of other targeted cross-links or bonds or other modifications, including but not limited to amino acid positions Y33, K77, N88, M97, A147, S150, S155, H159, N183, V185, V187, Y198, V220, E222, F223, K226, S255, Y286, K427 and V469, or any combination thereof; residue pairs 147/286, 198/220, 198/222, 198/223, 198/226, 33/496, 77/222, 88/255, 97/159,183/427, 185/427, and 187/427, or any combination thereof; regions or secondary structures including the F1 mobile N-terminus (residues 137-216), α2 (residues 148-160), α3 (residues 163-173), β3 (residues 176-182), β4 (residues 186-195), α4 (residues 197-211), the F1 mobile C-terminus (residues 461-513), β22 (residues 464-471), α9 (residues 474-479), β23 (residues 486-491), and α10 (493-514); and other regions of RSV F protein including residues from about position 67 to about position 87, from about position 78 to about position 98, from about position 87 to about position 107, from about position 137 to about position 157, from about position 140 to about position 160, from about position 145 to about position 165, from about position 149 to about position 169, from about position 173 to about position 193, from about position 175 to about position 195, from about position 177 to about position 197, from about position 188 to about position 208, from about position 210 to about position 230, from about position 212 to about position 232, from about position 213 to about position 233, from about position 216 to about position 236, from about position 245 to about position 265, from about position 276 to about position 296, from about position 417 to about position 437, and from about position 459 to about position 479.

A wide variety of methods of cross-linking proteins intra- and inter-molecularly are known in the art, including those having cross-links with varying lengths of spacer arms, and those with and without fluorescent and functional groups for purification. Such methods include, but are not limited to, the use of heterobifunctional cross-linkers (e.g. succinimidyl acetylthioacetate (SATA), trans-4-(maleimidylmethyl) cyclohexane-1-carboxylate (SMCC), and succinimidyl 3-(2-pyridyldithio) propionate (SPDP)), homobifunctional cross-linkers (e.g. succinimidyl 3-(2-pyridyldithio) propionate), photoreactive cross-linkers (e.g. 4-azido-2,3,5,6-tetrafluorobenzoic acid, STP ester, sodium salt (ATFB, STP ester), 4-azido-2,3,5,6-tetrafluorobenzoic acid, succinimidyl ester (ATFB, SE), 4-azido-2,3,5,6-tetrafluorobenzyl amine, hydrochloride, benzophenone-4-isothiocyanate, benzophenone-4-maleimide, 4-benzoylbenzoic acid, succinimidyl ester, N-((2-pyridyldithio)ethyl)-4-azidosalicylamide (PEAS; AET), thiol reactive cross-linkers (e.g. maleimides and iodoacetamides), amine reactive cross-linkers (e.g. glutaraldyde, bis(imido esters), bis(succinimidyl esters), diisocyanates and diacid chlorides). Because thiol groups are highly reactive and relatively rare in most proteins by comparison to amine groups, thiol-reactive cross-linking may be used in some embodiments. In cases where thiol groups are missing or not present at appropriate sites in the structures of RSV prefusion F protein, they can be introduced using one of several thiolation methods. For example, Succinimidyl trans-4-(maleimidylmethyl)cyclohexane-1-carboxylate can be used to introduce thiol-reactive groups at amine sites.

Several oxidative cross-links are known, such as disulfide bonds (which form spontaneously and are pH and redox sensitive), and di-tyrosine bonds (which are highly stable, and irreversible under physiological conditions).

In some embodiments the cross-links stabilize the tertiary structure of a RSV pre-fusion F protein. In some embodiments the cross-links stabilize the quaternary structure of a RSV pre-fusion F protein. In some embodiments the cross-links stabilize both the tertiary and quaternary structure of a RSV pre-fusion F protein.

In some embodiments a RSV F protein or polypeptide of the invention has cross-links that are thermostable. In some embodiments a RSV F protein or polypeptide of the invention has cross-links that are not toxic. In some embodiments a RSV F protein or polypeptide of the invention has cross-links that are targeted cross-links, or non-targeted cross-links, or reversible cross-links, or irreversible cross-links, or cross-links formed by use of homo-bifunctional cross-linking agents, or cross-links formed by use of hetero-bifunctional cross-linking agents, or cross-links formed by use of reagents that react with amine groups, or cross-links formed by use of reagents that react with thiol groups, or cross-links formed by use of reagents that are photoreactive, or cross-links formed between amino acid residues, or cross-links formed between mutated amino acid residues incorporated into the structure of the proteins or protein complexes, or oxidative cross-links, or di-tyrosine bonds, or glutaraldehdye cross-links, or any combination thereof. In some embodiments the RSV F protein or polypeptide of the invention does not have glutaraldehyde cross-links.

In some embodiments the RSV F protein or polypeptide of the invention does not have any artificially-introduced disulfide bonds, or if it does have such disulfide bonds, also has additional artificially-introduced cross-links. In some embodiments the RSV F protein or polypeptide of the invention does not have any artificially introduced disulfide bonds, but may have naturally occurring disulfide bonds. Disulfide bonds can be introduced artificially when cysteine side-chains are engineered by point mutation. Disulfide bonds are, however, known to be pH sensitive and to be dissolved under certain redox conditions, and the preventative and/or therapeutic utility of proteins and/or protein complexes engineered with disulfide cross-links, for example to be used as immunogens in vivo, may therefore be compromised. Furthermore, undesired disulfide bonds often form between proteins with free sulfhydryl groups that mediate aggregate formation (see, for example, Harris R J et al. 2004, Commercial manufacturing scale formulation and analytical characterization of therapeutic recombinant antibodies. Drug Dev Res 61 (3): 137-154; Costantino & Pikal (Eds.), 2004. Lyophilization of Biopharmaceuticals, editors Costantino & Pekal. Lyophilization of Biopharmaceuticals. Series: Biotechnology: Pharmaceutical Aspects II, see pages 453-454; Tracy et al., 2002, U.S. Pat. No. 6,465,425), which has also been reported as a problem with HIV gp120 and gp41 (Jeffs et al. 2004. Expression and characterization of recombinant oligomeric envelope glycoproteins derived from primary isolates of HIV-1. Vaccine 22:1032-1046; Schulke et al., 2002. Oligomeric and conformational properties of a proteolytically mature, disulfide-stabilized human immunodeficiency virus type 1 gp140 envelope glycoprotein. J Virol 76:7760-7776). Thus, in many embodiments it is preferred that disulfide bonding is not used, or is not used as the sole method of cross-linking.

If the structure and/or immunogenicity of the RSV prefusion F protein is compromised or altered by a cross-link, maintaining its overall structure and function can be achieved by controlling the availability of amino acid side-chains for the cross-linking reaction or by introducing additional cross-links or other stabilizing modifications. For example, in the case of DT cross-linking, tyrosyl side-chains that are available for reaction, but that lead to the distortion of the structure of the complex, and that compromise the immunogenicity/antigenicity of the RSV F protein, can be removed by mutating such residues to another amino acid such as, for example, phenylalanine. Furthermore, point mutations may be introduced at positions where the amino acid side-chains will react with cross-linking agents or each other, such that the formation of the bond(s) causes the most beneficial outcome. These positions may also be identified as described herein.

When at a selected residue a reactive side-chain is not already present, a point mutation may be introduced, for example using molecular biological methods to introduce such a point mutation into the cDNA of a nucleic acid directing its expression, such that a reactive side-chain is present and available for the reaction.

Cross-links that may be used include, but are not limited to, reversible cross-links resulting from the use of homo- and hetero-bifunctional cross-linking agents that react with amine and/or thiol groups, photoreactive cross-link reagents, any cross-links that may form between non-classical amino acids incorporated into the structure of a protein or protein complex, any oxidative cross-links, such as, but not limited to, di-tyrosine cross-links/bonds, heterobifunctional cross-linkers (e.g. succinimidyl acetylthioacetate (SATA), trans-4-(maleimidylmethyl)cyclohexane-1-carboxylate (SMCC), and succinimidyl 3-(2-pyridyldithio) propionate (SPDP)), homobifunctional cross-linkers (e.g. succinimidyl 3-(2-pyridyldithio) propionate), photoreactive cross-linkers (e.g. 4-azido-2,3,5,6-tetrafluorobenzoic acid, STP ester, sodium salt (ATFB, STP ester), 4-azido-2,3,5,6-tetrafluorobenzoic acid, succinimidyl ester (ATFB, SE), 4-azido-2,3,5,6-tetrafluorobenzyl amine, hydrochloride, benzophenone-4-isothiocyanate, benzophenone-4-maleimide, 4-benzoylbenzoic acid, succinimidyl ester, N-((2-pyridyldithio)ethyl)-4-azidosalicylamide (PEAS; AET), thiol reactive cross-linkers (e.g. maleimides and iodoacetamides), amine reactive cross-linkers (e.g. glutaraldyde, bis(imido esters), bis(succinimidyl esters), diisocyanates and diacid chlorides).

The present invention also contemplates the introduction of targeted non-covalent tyrosine-stacking interactions as “cross-links” to stabilize protein-protein interactions and/or desired protein or peptide conformations, such as the pre-fusion conformation of RSV F protein. The cross-link comprises a targeted pi stacking interaction including but not limited to a T-shaped, sandwich, or parallel displaced pi stacking interaction between the aromatic side chains of an introduced/engineered tyrosine and an endogenous tyrosine, phenylalanine, histidine, or tryptophan within the protein or protein complex, or between the aromatic side chain of an introduced/engineered tyrosine and a second introduced/engineered tyrosine within the protein or protein complex.

Irreversible cross-links, as used in the context of this application, include those that are not significantly dissolved under physiologically relevant conditions. It is preferred that the type of cross-links used should not lead to aggregate formation during expression or when the RSV F polypeptides, proteins and/or protein complexes of the invention are stored at high concentrations. Disulfide bonds are not irreversible cross-links. Rather they are reversible cross-links and may dissolve under physiologically relevant conditions and/or lead to aggregate formation during protein expression and/or production or when stored in high concentrations.

In some embodiments cross-links may be targeted to the specific regions of RSV F polypeptides, proteins and/or protein complexes described herein in order to achieve the desired conformational stabilization and/or the desired immunogenic properties (e.g. the ability to maintain the pre-F conformation and/or to bind to broadly neutralizing antibodies). Alternatively, proteins with the cross-links at the locations specified herein may be isolated from a mixture of cross-linked and un-cross-linked proteins with and without desired modifications, for example based on chemical, physical, and/or functional characteristics. Such characteristics may include, for example, trimerization, the presence of the pre-F conformation, and/or any desired antigenic, immunogenic, or biochemical characteristics.

Alternatively, in some embodiments, cross-links may not be targeted, and proteins with the desired cross-links or properties may be isolated from a mixture of modified and unmodified proteins made using a non-targeted cross-linking system.

In embodiments where RSV F polypeptides, proteins or protein complexes with the desired cross-links are to be isolated from a mixture of cross-linked and un-cross-linked proteins, such isolation or separation may be performed on the basis of one or more characteristics including, but not limited to, molecular weight, molecular volume, chromatographic properties, mobility in electrophoresis, antigenic and biochemical characteristics, fluorescence characteristics, solubility, binding to antibodies, structural characteristics, immunological characteristics, or any other suitable characteristics.

In addition to the specific cross-linking positions described herein, additional positions within RSV F polypeptides, proteins or protein complexes can be identified at which further cross-links can be made, for example where a reactive side-chain would be able to form a bond with a reactive side-chain elsewhere on the RSV F protein/complex. In some embodiments, such additional positions can be selected, for example, to maintain or improve the immunogenicity/antigenicity of the protein, polypeptide or protein complex. In some embodiments, such additional positions to be cross-linked can be selected in pairs.

### Di-tyrosine (DT) Cross-Linking

In some embodiments the present invention provides RSV F polypeptides, proteins and/or protein complexes that comprise di-tyrosine (DT) cross-links, and methods of making such DT-cross-linked RSV F polypeptides, proteins and/or protein complexes.

Di-tyrosine cross-linking introduces one or more covalent carbon-carbon bonds into proteins or protein complexes. This provides a method for stabilizing proteins, protein complexes, and conformations, by introduction of intra- and/or inter-polypeptide di-tyrosine bonds while maintaining their structural and functional integrity (See Marshall et al., U.S. Pat. Nos. 7,037,894 & 7,445,912, the contents of which are hereby incorporated by reference). The minimally altering, and zero-length DT cross-link is not hydrolyzed under physiological conditions, and has been demonstrated to maintain proteins' structural integrity by liquid chromatography/mass spectrometry (LC/MS). Di-tyrosine cross-links are known to be safe, as they form naturally in vivo, both in the context of proteins evolved to utilize their specific characteristics (e.g. Elvin C M et al. 2005, Nature 437:999-1002; Tenovuo J & Paunio K 1979, Arch Oral Biol.; 24 (8): 591-4), and as a consequence of non-specific protein oxidation (Giulivi et al. 2003, Amino Acids 25 (3-4): 227-32), and as they are present in large quantities in some of our most common foods: DT bonds form the structure of wheat gluten—the quarternary protein structure comprising the glutenin subunits—e.g. in bread dough during mixing and baking (Tilley et al. 2001, Agric. Food Chem 49, 2627). Di-tyrosine bonds do not form spontaneously in vitro. Rather, the enzymatic cross-link reaction is carried out under optimized conditions to preserve protein structure and function. Therefore, non-specific bonding/aggregation does not occur (unlike with disulfide bonding), and therefore large-scale manufacturing of a DT stabilized immunogen may be economically more feasible.

Tyrosyl side-chains are present in many redox enzymes, and catalysis of the enzyme-specific reactions often involves tyrosyl radicals that are long-lived and have comparatively low reactivity. Under optimized conditions radical formation is specific to tyrosyl side-chains. In close proximity, tyrosyl side chains undergo radical coupling and form a covalent, carbon-carbon bond. Tyrosyl radicals that do not react revert to non-radicalized tyrosyl side-chains (Malencik & Anderson, 2003. Di-tyrosine as a product of oxidative stress and fluorescent probe. Amino Acids 25:233-247). Therefore, tyrosyl side-chains must be situated in close proximity to form DT bonds, either within a single folded polypeptide chain, or on closely interacting protein domains within a complex. Because a Cα-Cα separation of approximately 5-8 Å is a prerequisite to bond formation (Brown et al., 1998. Determining protein-protein interactions by oxidative cross-linking of a glycine-glycine-histidine fusion protein. Biochemistry 37, 4397-4406; Marshall et al. 2006, U.S. Pat. No. 7,037,894), and because no atom is added in the formation of these bonds, the resulting “staple” is “zero length” and non-disruptive to the protein structure.

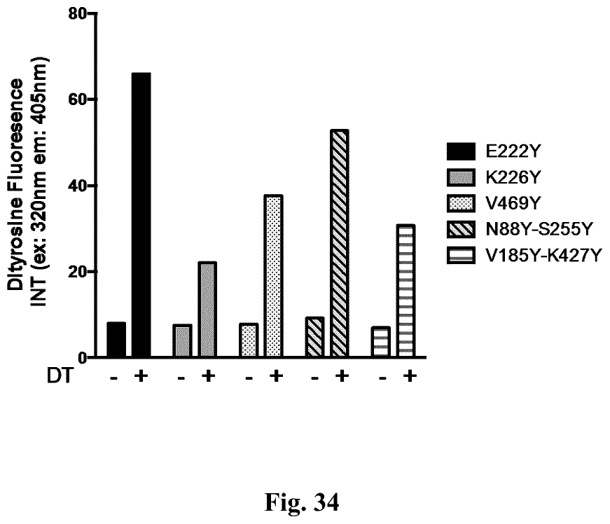

Tyrosines residues to be cross-linked may be naturally present in the primary structure of the protein to be cross-linked or may be added by controlled point mutation. To form DT bonds, proteins with tyrosyl side chains can be subjected to reaction conditions that lead to the formation of DT bonds. Such conditions are, or become, oxidative reaction conditions, as the DT bond formation reaction is an oxidative cross-linking reaction. In some embodiments the DT cross-linking reaction conditions yield proteins that are otherwise not, or not detectably, modified. Such conditions may be obtained by use of enzymes that catalyze the formation of H₂O₂, such as peroxidases. DT bond formation may be monitored by spectrophotometry with an excitation wavelength of around 320 nm, and fluorescence measured at a wavelength of around 400 nm (see, for example, FIG. 34), while loss of tyrosyl fluorescence is monitored also monitored by standard procedures. When loss of tyrosyl florescence is no longer stoichiometric with DT bond formation, the reaction may be stopped by any methods known to one skilled in the art, such as, for example, by the addition of a reducing agent and subsequent cooling (on ice) or freezing of the sample. Further details of how to perform DT cross-linking are known in the art and are described in, for example, Marshall et al. 2006, U.S. Pat. No. 7,037,894, the contents of which are hereby incorporated by reference.

The major advantages of di-tyrosine cross-linking in protein engineering include (i) the ability to target specific residues for cross-linking (based on the primary, secondary, tertiary, and/or quaternary structures of proteins and complexes), (ii) minimal structural modification, (iii) specificity of the reaction (tyrosine is the only amino acid known to form cross-links under specific cross-linking conditions); (iv) stability of the linkage, (v) zero length of the cross-link (no atom is added), and (vi) the scalability of the cross-linking chemistry.

In some embodiments, targeted DT cross-links may be introduced at one or more of the specific locations in the RSV F protein that are recited herein. In other embodiments, additional positions within RSV F polypeptides, proteins or protein complexes can be identified at which DT cross-links can be made. In some embodiments, di-tyrosine bonds or cross-links are targeted to specific residue pairs within the structure of a RSV F protein or polypeptide where DT bonds will, or are expected to, form due to, for example, their close proximity. In some embodiments tyrosyl side chains are already present at amino acid residues to be cross-linked. In some cases naturally occurring tyrosine residues may constitute either one or both of the paired tyrosine residues necessary for di-tyrosine bond formation. However, in other cases the RSV F polypeptides, proteins and/or protein complexes of the invention are mutated or engineered to add one or more tyrosine residues, or to substitute one or more non-tyrosine residues for tyrosine residues. Such mutations are referred to herein as “to-tyrosine” mutations, and can be introduced at locations where it is desirable to form di-tyrosine cross-links/bonds. In some embodiments, the present invention provides mutant RSV F polypeptides, proteins, and/or protein complexes in which tyrosyl side chains are introduced at desired cross-linking positions by introducing point mutations to tyrosine in a nucleic acid sequence encoding the RSV F polypeptide, protein, or protein complex. Alternatively, in some embodiments RSV proteins, polypeptides or protein complexes, or portions thereof, may be synthesized to include tyrosine residues or amino acids having tyrosyl side chains at desired cross-linking positions. Conversely, in some embodiments the present invention provides mutant RSV F polypeptides, proteins, and/or protein complexes in which tyrosyl side chains are removed at undesirable cross-linking positions by introducing point mutations from tyrosine in a nucleic acid sequence encoding the RSV F polypeptide, protein, or protein complex, or RSV F polypeptides, proteins, or protein complexes may be synthesized to exclude tyrosine residues or amino acids having tyrosyl side chains at positions where cross-linking is not desired. For example, at least one of the tyrosyl side chains can be replaced with another side chain, such as a phenylalanine side chain (see, for example, Marshall C P et al., U.S. patent application Ser. No. 09/837,235, the contents of which are hereby incorporated by reference). Accordingly, the RSV F polypeptides, proteins and protein complexes of the invention may comprise point mutations “to tyrosine” or “from tyrosine.” Such mutations can be made by altering the nucleic acid sequences that encode the RSV F polypeptides, proteins and/or protein complexes of the invention using any suitable mutagenesis methods known in the art. Alternatively, mutant RSV F polypeptides, proteins and/or protein complexes may be synthesized, purified, and/or produced by any other suitable methods known in the art.

In some embodiments, the present invention contemplates the targeted introduction of one or more di-tyrosine cross-link at any suitable position(s) in a RSV F polypeptide, protein or protein complex where the cross-link will or may stabilize the RSV F polypeptide, protein or protein complex in its pre-fusion conformation. Such stabilization may be achieved, for example, by introducing cross-links that stabilize interactions between or within RSV F protein F1 and F2 polypeptides and/or by introducing cross-links that stabilize the interactions between or within RSV F protein protomers. In some embodiments, the F1 polypeptide of a RSV F protein is cross-linked with the F2 polypeptide of the same protomer (inter-molecular/intra-protomer bond). In some embodiments, the F1 polypeptide is intra-molecularly cross-linked (e.g., both tyrosines of the cross-link are located within the same F1 polypeptide). In some embodiments, the F2 polypeptide is intra-molecularly cross-linked (e.g., both tyrosines of the cross-link are located within the same F1 polypeptide). In some embodiments, the F1 polypeptide of the RSV prefusion F protein is cross-linked with the F1 polypeptide of an adjacent protomer (inter-protomer bond). In some embodiments, the F1 polypeptide of the RSV prefusion F protein is cross-linked with the F2 polypeptide of an adjacent protomer (inter-protomer bond).

### Making and Analyzing RSV F Polypeptides, Proteins, and Protein Complexes

In some embodiments the present invention provides methods for making the RSV F polypeptides, proteins, and protein complexes of the invention. The RSV F polypeptides, proteins, and protein complexes of the invention can be made by any suitable means known in the art. In some embodiments the RSV F polypeptides, proteins, and/or protein complexes of the invention can be made by recombinant means. In some embodiments, the RSV F polypeptides, proteins, and/or protein complexes of the invention, or any portion thereof, can be made by chemical synthesis means. For example, a peptide corresponding to a portion of a protein or protein complex as described herein can be synthesized by use of a peptide synthesizer.

### Recombinant Production Methods

In embodiments where the RSV F polypeptides, proteins and protein complexes of the invention are made by recombinant means, nucleic acids encoding the RSV F polypeptides, proteins and protein complexes of the invention can be expressed in any suitable cell type, including, but not limited to mammalian cells and insect cells (such as SF9 or Hi5 cells, using a baculovirus expression system). Methods for expressing polypeptides and proteins from nucleic acid molecules are routine and well known in the art, and any suitable methods, vectors, systems, and cell types known in the art can be used. For example, typically nucleic acid sequences encoding the RSV F polypeptides, proteins and/or protein complexes of the invention will be placed into a suitable expression construct containing a suitable promoter, which will then be delivered to cells for expression.

### Chimeric/Fusion Proteins & Oligomerization Domains

In some embodiments it may be desirable to add chimeric domains to the RSV F polypeptides, proteins and/or protein complexes described herein, to produce chimeric proteins/fusion proteins, for example to facilitate the analysis and/or isolation and/or purification of the RSV F polypeptides, proteins and/or protein complexes described herein. In some embodiments, the RSV F polypeptides, proteins and protein complexes of the invention may comprise leader sequences, precursor polypeptide sequences, secretion signals, localization signals, epitope tags, and the like. Epitope tags that can be used include, but are not limited to, FLAG tags, glutathione S-transferase (GST) tags, green fluorescent protein (GFP) tags, hemagglutinin A (HA) tags, histidine (His) tags, luciferase tags, maltose-binding protein (MBP) tags, c-Myc tags, protein A tags, protein G tags, streptavidin (strep) tags, and the like.

In some embodiments it may be desirable to add oligomerization domains to facilitate the assembly of RSV F polypeptides, proteins and/or protein complexes as described herein, and/or to facilitate stabilization of the pre-F conformation, and/or to stabilize other structural features of the RSV F polypeptides, proteins and/or protein complexes. In some embodiments the oligomerization domains are trimerization motifs, including, but not limited to, the T4 foldon motif. There are a wide variety of trimerization domains in natural proteins that can be used for these purposes including, but not limited to, those described in Habazettl et al., 2009 (Habazettl et al., 2009. NMR Structure of a Monomeric Intermediate on the Evolutionarily Optimized Assembly Pathway of a Small Trimerization Domain. J. Mol. Biol. pp. null), Kammerer et al., 2005. (Kammerer et al., 2005. A conserved trimerization motif controls the topology of short coiled coils. Proc Natl Acad Sci USA 102 (39): 13891-13896), Innamorati et al., 2006. (Innamorati et al., 2006. An intracellular role for the Clq-globular domain. Cell signal 18 (6): 761-770), and Schelling et al., 2007 (Schelling et al., 2007. The reovirus σ-1 aspartic acid sandwich: A trimerization motif poised for conformational change. Biol Chem 282 (15): 11582-11589). Stabilizing trimeric protein complexes can also be accomplished using the GCN4 and T4 fibrinitin motifs (Pancera et al., 2005. Soluble Mimetics of Human Immunodeficiency Virus Type 1 Viral Spikes Produced by Replacement of the Native Trimerization Domain with a Heterologous Trimerization Motif: Characterization and Ligand Binding Analysis. J Virol 79 (15): 9954-9969; Guthe et al., 2004. Very fast folding and association of a trimerization domain from bacteriophage T4 fibritin. J. Mol. Biol. v337 pp. 905-15; Papanikolopoulou et al., 2008. Creation of hybrid nanorods from sequences of natural trimeric fibrous proteins using the fibritin trimerization motif. Methods Mol Biol 474:15-33). Heterologous oligomerization motifs may be introduced by any recombinant methods known to one of ordinary skill in the art in order to stabilize the protein-protein interactions of the proteins of the present invention.

Chimeric RSV F polypeptides, proteins and/or protein complexes can be made by any method known to one of ordinary skill in the art, and may comprise, for example, one or several RSV F polypeptides, proteins and/or protein complexes of the invention, and/or any fragment, derivative, or analog thereof (for example, consisting of at least a domain of a polypeptide, protein, or protein complex of the invention, or at least 6, and preferably at least 10 amino acids of thereof) joined at its amino- or carboxy-terminus via a peptide bond to an amino acid sequence of another protein or other protein domain or motif. In some embodiments such chimeric proteins can be produced by any method known to one of ordinary skill in the art, including, but not limited to, recombinant expression of a nucleic acid encoding a chimeric protein (e.g. comprising a first coding sequence joined in-frame to a second coding sequence); ligating the appropriate nucleic acid sequences encoding the desired amino acid sequences to each other in the proper coding frame, and expressing the chimeric product.

### Post Translational Modifications

In some embodiments, the RSV F polypeptides, proteins and protein complexes described herein may be altered by adding or removing post-translational modifications, by adding or removing chemical modifications or appendices, and/or by introducing any other modifications known to those of ordinary skill in the art. Included within the scope of the invention are RSV F polypeptides, proteins and protein complexes that are modified during or after translation or synthesis, for example, by glycosylation (or deglycosylation), acetylation (or deacetylation), phosphorylation (or dephosphorylation), amidation (or deamidization), pegylation, derivatization by known protecting/blocking groups, proteolytic cleavage, or buy any other means known in the art. For example, in some embodiments the RSV F polypeptides, proteins and/or protein complexes may be subjected to chemical cleavage by cyanogen bromide, trypsin, chymotrypsin, papain, V8 protease, NaBH4, acetylation, formylation, oxidation, reduction, metabolic synthesis in the presence of tunicamycin, etc. In some embodiments such posttranslational modifications can be used to render the RSV F polypeptides, proteins, and/or protein complexes of the present invention more immunogenic, more stable, and/or more capable of binding to, or eliciting the production of, neutralizing and broadly neutralizing antibodies.

### Obtaining RSV F in its Pre-F Conformation

In some embodiments the RSV F polypeptides and/or proteins of the invention are assembled into protein complexes having a desired conformational structure, such as the pre-F conformation, and are cross-linked in order to stabilize that conformation. As described elsewhere in the present application, the pre-F conformation of the RSV F protein comprises a trimer formed from three protomers. In some embodiments, prior to and/or during the enzymatic cross-linking reaction, the RSV F protein may be obtained in (and/or maintained in) the pre-F conformation, for example while cross-linking is performed. In some embodiments the RSV F protein may be produced and/or isolated in such a way that most, or substantially all, of the RSV F molecules are present in the pre-F conformation. In some embodiments RSV F molecules in the pre-F conformation may be separated from a mixed population of RSV F protein molecules comprising some that are in the pre-F conformation and some that are in other conformations. In some embodiments, the RSV F protein is expressed in cells (for example as its membrane bound or soluble form) and spontaneously assembles into its normal pre-F conformation. In some embodiments no additional stabilization may be necessary to retain the RSV F protein in its pre-F form. In some embodiments the expressed and assembled/folded RSV F protein may be kept under particular conditions, or in particular compositions, that favor formation and/or maintenance of the pre-F conformation. For example, in some embodiments the RSV prefusion F protein may be maintained in the absence of cells-contact with which might otherwise trigger a switch to the post-F conformation. The RSV prefusion F protein may be obtained and/or isolated and/or maintained in the pre-F conformation using any suitable method known in the art, including, but not limited to, standard protein purification methods, such as ion exchange chromatography, size exclusion chromatography, and/or affinity chromatography methods. In some embodiments the RSV prefusion F protein may be expressed in the presence of, co-expressed with, or contacted with, molecules that bind to the RSV F protein and stabilize it in its pre-F conformation, including, but not limited to, antibodies, small molecules, peptides, and/or peptidomimetics. Non-limiting examples of antibodies that bind to the pre-fusion RSV F protein include the 5C4, AM22, and D25 antibodies (see Mclellan et al. (2013) Science 342:592-598, which is hereby incorporated by reference in its entirety). In some embodiments, the RSV F protein may be obtained, isolated, or maintained in its pre-F conformation by controlling the ionic strength of the media/buffer in which the protein is present (such as by using high or low ionic strength media). In some embodiments the RSV F protein may be obtained, isolated, or maintained at one or more temperatures that favor preservation of the pre-F conformation. In some embodiments the RSV F protein may be obtained, isolated, or maintained over a period of time that diminishes the degree to which the pre-F conformation lost.

In some embodiments analysis may be performed to confirm that the desired conformation, such as the pre-F conformation, has been formed and/or maintained in the RSV F protein. Such analysis may be performed prior to cross-linking, during the cross-linking process, after the cross-linking process, or at any combination of such stages. Such analysis may comprise any suitable methods known in the art for assessing the 3-dimensional structure of a protein or protein complex, including functional analysis, crystallographic analysis, and the like. In some embodiments such analysis may include assessing binding of the RSV protein to certain antibodies, such as those that are specific to the pre-F conformation and/or those that are known to bind to the ø site, as described elsewhere herein, including, but not limited to the 5C4, AM22, and D25 antibodies.

### Protein Purification

In some embodiments the methods for making RSV F polypeptides, proteins, and protein complexes of the invention may comprise purifying the RSV F polypeptides, proteins, or protein complexes before, during, or after, one or more steps in the manufacturing process. For example, in some embodiments the RSV F polypeptides, proteins, and/or protein complexes of the invention may be purified after completion of all of the manufacturing steps. In some embodiments the RSV F polypeptides, proteins, and/or protein complexes of the invention may be purified before commencing the cross-linking process or after one or more of the intermediate method steps in the process, for example, after expression of an RSV F polypeptide or protein, after assembly of a protein complex, after obtaining the RSV F protein in its pre-F conformation, or during or after performing a cross-linking reaction. The RSV F polypeptides, proteins, and/or protein complexes of the invention may be isolated or purified using any suitable method known in the art. Such methods include, but are not limited to, chromatography (e.g. ion exchange, affinity, and/or sizing column chromatography), ammonium sulfate precipitation, centrifugation, differential solubility, or by any other technique for the purification of proteins known to one of ordinary skill in the art. In specific embodiments it may be necessary to separate the desirable influenza RSV F polypeptides, proteins, and/or protein complexes of the invention from those that were not sufficiently cross-linked, or those in which the pre-F conformation was not sufficiently stabilized. This can be done using any suitable system known in the art. For example, RSV proteins in the pre-F conformation can be separated from those that are not in the pre-F conformation using antibody-based separation methods using pre-F or post-F specific antibodies. The RSV F polypeptides, proteins, and/or protein complexes of the invention may be purified from any source used to produce them. For example, the RSV F polypeptides, proteins, and/or protein complexes of the invention may be purified from sources including insect, prokaryotic, eukaryotic, mono-cellular, multi-cellular, animal, plant, fungus, vertebrate, mammalian, human, porcine, bovine, feline, equine, canine, avian, or tissue culture cells, or any other source. The degree of purity may vary, but in various embodiments, the purified RSV F polypeptides, proteins, and/or protein complexes of the invention are provided in a form in which is they comprise more than about 10%, 20%, 50%, 70%, 75%, 80%, 85%, 90%, 95%, 98%, 99%, or 99.9% of the total protein in the final composition. In some embodiments the RSV F polypeptides, proteins, and/or protein complexes of the invention may be isolated and purified from other proteins, or any other undesirable products (such as non-cross-linked or non-pre-F RSV F), by standard methods including, but not limited to, chromatography, glycerol gradients, affinity chromatography, centrifugation, ion exchange chromatography, size exclusion chromatography, and affinity chromatography, or by any other standard technique for the purification of proteins known in the art. The RSV F polypeptides, proteins, and/or protein complexes to be isolated may be expressed in high or low ionic media, or isolated in high or low ionic buffers or solutions. The RSV F polypeptides, proteins, and/or protein complexes of the invention may also be isolated at one or more temperatures that favor preservation of the desired conformation. They may also be isolated over a period of time that diminishes the degree to which a preparation would have lost the desired conformation. The degree to which a preparation of proteins retains one or more desired conformations (such as the pre-F conformation and/or conformations that favor binding to neutralizing antibodies, or the desired properties) may be assayed by any suitable method known in the art, including, for example, but not limited to, biochemical, biophysical, immunologic, and virologic analyses. Such assays include, for example, but are not limited to, immunoprecipation, enzyme-linked immunosorbent assays (ELISAs), or enzyme-linked immunosorbent spot (ELISPOT) assays, crystallographic analysis (including co-crystallization with antibodies), sedimentation, analytical ultracentrifugation, dynamic light scattering (DLS), electron microscopy (EM), cryo-EM tomography, calorimetry, surface plasmon resonance (SPR), fluorescence resonance energy transfer (FRET), circular dichroism analysis, and small angle x-ray scattering, neutralization assays, antibody-dependent cellular cytotoxicity assays, and/or virologic challenge studies in vivo.

The yield of the RSV F polypeptides, proteins, and/or protein complexes of the invention can be determined by any means known in the art, for example, by comparing the amount of the final engineered proteins (such as cross-linked pre-F RSV) as compared to the amount of the starting material, or as compared to the amount of the materials present in any preceding step of the production methods. Protein concentrations can determined by standard procedures, such as, for example, Bradford or Lowrie protein assays. The Bradford assay is compatible with reducing agents and denaturing agents (Bradford, M, 1976. Anal. Biochem. 72:248). The Lowry assay has better compatibility with detergents and the reaction is more linear with respect to protein concentrations and read-out (Lowry, O J, 1951. Biol. Chem. 193:265).

### Exemplary Production Methods

In some embodiments the present invention provides a method for producing a RSV F protein stabilized in its pre-fusion conformation, the method comprising: (a) obtaining an RSV F protein in its pre-F conformation, (b) identifying one or more regions in the tertiary and/or quaternary structure of the RSV prefusion F protein in which the introduction of one or more cross-links could stabilize the pre-F conformation, and (c) introducing into the RSV prefusion F protein one or more cross-links at one or more of the regions identified in step (b) to form an engineered RSV F protein locked in its pre-fusion conformation. In some embodiments, the regions identified in step (b) comprise one or more of the specific regions or specific amino acid residues described herein. In some embodiments the cross-links are targeted cross-links. In some embodiments the cross-links are targeted DT cross-links. In some embodiments the cross-links are stable under physiological conditions. In some embodiments, the engineered RSV F protein stabilized in its pre-fusion conformation is useful as a vaccine immunogen. In some embodiments, the engineered RSV F protein locked in its pre-fusion conformation has one or more of the following properties: (i) enhanced ability bind to a neutralizing antibody as compared to the RSV F protein not so engineered (i.e. as compared to the RSV F protein without or before introduction of the cross-links), (ii) enhanced ability bind to a broadly neutralizing antibody as compared to the RSV F protein not so engineered, (iii) enhanced ability bind to and activate B cell receptors as compared to the RSV F protein not so engineered, (iv) enhanced ability to elicit an antibody response in an animal as compared to the RSV F protein not so engineered, (v) enhanced ability to elicit a protective antibody response in an animal as compared to the RSV F protein not so engineered, (vi) enhanced ability to elicit production of neutralizing antibodies in an animal as compared to the RSV F protein not so engineered, (vii) enhanced ability to elicit production of broadly neutralizing antibodies in an animal as compared to the RSV F protein not so engineered, (viii) enhanced ability to elicit a protective immune response in an animal as compared to the RSV F protein not so engineered, and (ix) enhanced ability to bind to and elicit production of antibodies that recognize quaternary neutralizing epitopes in an animal as compared to the RSV F protein not so engineered. In some embodiments the methods for producing an RSV F protein stabilized in its pre-fusion conformation described herein also comprise performing an assay to determine if the engineered RSV F protein stabilized in its pre-fusion conformation and/or has one or more of the properties listed above.

Properties of RSV F Polypeptides, Proteins and/or Protein Complexes

In some embodiments, the RSV F polypeptides, proteins and/or protein complexes of the invention, including in particular those that are cross-linked as described herein, have certain structural, physical, functional, and/or biological properties. Such properties may include one or more of the following, or any combination of the following: existence of the pre-F conformation, stability of the RSV pre-F conformation; Improved stability of the RSV pre-F conformation (as compared to non-cross-linked RSV F proteins); Improved half-life of the RSV pre-F conformation (as compared to non-cross-linked RSV F proteins); Improved thermostability (as compared to non-cross-linked RSV F proteins); Prolonged shelf-life (as compared to non-cross-linked RSV F proteins); Prolonged half-life inside the body of a subject (as compared to non-cross-linked RSV F proteins); Ability to be stored in solution without forming aggregates (including when present at a high concentration in solution); Reduced aggregation in solution (as compared to non-cross-linked RSV F proteins); Binding to an antibody; Binding to a neutralizing antibody; Binding to a broadly neutralizing antibody; Binding to a pre-F-specific antibody; Binding to an antibody that recognizes site ø; Binding to a conformationally-specific antibody; Binding to an antibody that recognizes a metastable epitope; Binding to an antibody selected from the group consisting of D25, AM22 and 5C4 (which antibodies are described in McLellan et al., 2013, Science, 340, p. 1113; Kwakkenbos et al., 2010, Nature Medicine, 16, p. 123; Spits & Beaumont, U.S. patent application Ser. No. 12/600,950; Beaumont, Bakker & Yasuda, U.S. patent application Ser. No. 12/898,325, the contents of each of which are hereby incorporated by reference in their entireties); Binding to palivizumab (Synagis); Binding to the neutralizing antibody 101F; Binding to a B cell receptor; Activation of a B Cell receptor; Eliciting an antibody response in an animal; Eliciting a protective antibody response in an animal; Eliciting production of neutralizing antibodies in an animal; Eliciting production of broadly neutralizing antibodies in an animal; Eliciting production of antibodies that recognize quaternary neutralizing epitopes (QNEs) in an animal; Eliciting a protective immune response in an animal; and/or Eliciting a humoral immune response in an animal. In the case of binding to antibody molecules, in some embodiments the RSV F polypeptides, proteins, and/or protein complexes of the invention bind to the antibodies (such as pre-F-specific antibodies, antibodies that bind to site ø, and/or D25, AM22 or 5C4) with high specificity and/or with high affinity.

### Assays for Properties

In some embodiments the RSV F polypeptides, proteins, and/or protein complexes of the invention, or any intermediates in their manufacture, may be analyzed to confirm that they have desired properties, such as the desired structural, physical, functional, and/or biological properties-such as those properties listed above or identified elsewhere in this patent specification. For example, in some embodiments in vitro or in vivo assays can be performed to assess the RSV F protein's conformational structure, stability (e.g. thermostability), half-life (e.g. inside the body of a subject), aggregation in solution, binding to an antibody (such as a neutralizing antibody, broadly neutralizing antibody; pre-F-specific antibody; antibody that recognizes site ø, conformationally-specific antibody, antibody that recognizes a metastable epitope, D25, AM22, 5C4, 101F or palivizumab), binding to a B cell receptor, activation of a B Cell receptor, antigenicity, immunogenicity, ability to elicit an antibody response, ability to elicit a protective antibody/immune response, ability to elicit production of neutralizing antibodies, or ability to elicit a production of broadly neutralizing antibodies. In embodiments where the RSV F polypeptides, proteins, and/or protein complexes of the invention are tested in an animal in vivo, the animal may be any suitable animal species, including, but not limited to a mammal (such as a rodent species (e.g. a mouse or rat), a rabbit, a ferret, a porcine species, a bovine species, an equine species, an ovine species, or a primate species (e.g. a human or a non-human primate), or an avian species (such as a chicken).

Assays for assessing a protein's conformational structure are well known in the art and any suitable assay can be used, including, but not limited to, crystallographic analysis (e.g. X-ray crystallography or electron crystallography), sedimentation analysis, analytical ultracentrifugation, electron microscopy (EM), cryo-electron microscopy (cryo-EM), cryo-EM tomography, nuclear magnetic resonance (NMR), small angle x-ray scattering, fluorescence resonance energy transfer (FRET) assays, and the like.

Assays for assessing a protein's stability are well known in the art and any suitable assay can be used, including, but not limited to, denaturing and non-denaturing electrophoresis, isothermal titration calorimetry, and time-course experiments in which proteins are incubated and analyzed over time at varying protein concentrations, temperatures, pHs or redox conditions. Proteins may also be analyzed for susceptibility to proteolytic degradation.

Assays for assessing binding of proteins to antibodies are well known in the art, and any suitable assay can be used, including, but not limited to, immunoprecipation assays, enzyme-linked immunosorbent assays (ELISAs), enzyme-linked immunosorbent spot assays (ELISPOTs), crystallographic assays (including co-crystallization with antibodies), surface plasmon resonance (SPR) assays, fluorescence resonance energy transfer (FRET) assays, and the like.

Assays for assessing neutralization activity are well known in the art, and any suitable assay can be used. For example, assays can be performed to determine the neutralizing activity of antibodies or antisera generated by vaccination/immunization of animals with the RSV F polypeptides, proteins, and/or protein complexes of the invention. Neutralization assays known in the art include, but are not limited to, those described by Dey et al. 2007 (Dey et al., 2007, Characterization of Human Immunodeficiency Virus Type 1 Monomeric and Trimeric gp120 Glycoproteins Stabilized in the CD4-Bound State: Antigenicity, Biophysics, and Immunogenicity. J Virol 81 (11): 5579-5593) and Beddows et al., 2006 (Beddows et al., 2007, A comparative immunogenicity study in rabbits of disulfide-stabilized proteolytically cleaved, soluble trimeric human immunodeficiency virus type 1 gp140, trimeric cleavage-defective gp140 and monomeric gp120. Virol 360:329-340).

Assays for assessing whether a vaccine immunogen is capable of eliciting an immune response and/or proving protective immunity are well known in the art, and any suitable assay can be used. For example, assays can be performed to determine whether vaccination/immunization of animals with the RSV F polypeptides, proteins, and/or protein complexes of the invention provide an immune response and/or protective immunity against infection with RSV. In some embodiments comparisons may be made between placebo and test vaccinated groups with regard to their rates of infection or sero-conversion or viral loads.

Assays for assessing a protein's pharmacokinetics and bio-distribution are also well known in the art, and any suitable assay can be used to assess these properties of the the RSV F polypeptides, proteins, and/or protein complexes of the invention.

### Compositions

In some embodiments the present invention provides compositions comprising any of the RSV F polypeptides, proteins, and/or protein complexes described herein. In some embodiments such compositions may be immunogenic compositions, vaccine compositions and/or therapeutic compositions. In some embodiments, such compositions may be administered to subjects.

In some embodiments the RSV F polypeptides, proteins, and/or protein complexes of the invention may be provided in a composition that comprises one or more additional active components, such as one or more additional vaccine immunogens or therapeutic agents. In some embodiments the RSV F polypeptides, proteins, and/or protein complexes of the invention may be provided in a composition that comprises one or more other components, including, but not limited to, pharmaceutically acceptable carriers, adjuvants, wetting or emulsifying agents, pH buffering agents, preservatives, and/or any other components suitable for the intended use of the compositions. Such compositions can take the form of solutions, suspensions, emulsions and the like. The term “pharmaceutically acceptable carrier” includes various diluents, excipients and/or vehicles in which, or with which, the RSV F polypeptides, proteins, and/or protein complexes of the invention can be provided. The term “pharmaceutically acceptable carrier” includes, but is not limited to, carriers known to be safe for delivery to human and/or other animal subjects, and/or approved by a regulatory agency of the Federal or a state government, and/or listed in the U.S. Pharmacopeia, and/or other generally recognized pharmacopeia, and/or receiving specific or individual approval from one or more generally recognized regulatory agencies for use in humans and/or other animals. Such pharmaceutically acceptable carriers, include, but are not limited to, water, aqueous solutions (such as saline solutions, buffers, and the like), organic solvents (such as certain alcohols and oils, including those of petroleum, animal, vegetable or synthetic origin, such as peanut oil, soybean oil, mineral oil, sesame oil), and the like. In some embodiments the compositions of the invention also comprise one or more adjuvants. Exemplary adjuvants include, but are not limited to, inorganic or organic adjuvants, oil-based adjuvants, virosomes, liposomes, lipopolysaccharide (LPS), molecular cages for antigens (such as immune-stimulating complexes (“ISCOMS”)), Ag-modified saponin/cholesterol micelles that form stable cage-like structures that are transported to the draining lymph nodes), components of bacterial cell walls, endocytosed nucleic acids (such as double-stranded RNA (dsRNA), single-stranded DNA (ssDNA), and unmethylated CpG dinucleotide-containing DNA), AUM, aluminum phosphate, aluminum hydroxide, and Squalene. In some embodiments virosomes are used as the adjuvant. Additional commercially available adjuvants that can be used in accordance with the present invention include, but are not limited to, the Ribi Adjuvant System (RAS, an oil-in-water emulsion containing detoxified endotoxin (MPL) and mycobacterial cell wall components in 2% squalene (Sigma M6536)), TiterMax (a stable, metabolizable water-in-oil adjuvant (CytRx Corporation 150 Technology Parkway Technology Park/Atlanta Norcross, Georgia 30092)), Syntex Adjuvant Formulation (SAF, an oil-in-water emulsion stabilized by Tween 80 and pluronic polyoxyethlene/polyoxypropylene block copolymer L121 (Chiron Corporation, Emeryville, CA)), Freund's Complete Adjuvant, Freund's Incomplete Adjuvant, ALUM-aluminum hydroxide, Al(OH)3 (available as Alhydrogel, Accurate Chemical & Scientific Co, Westbury, NY), SuperCarrier (Syntex Research 3401 Hillview Ave. P.O. Box 10850 Palo Alto, CA 94303), Elvax 40W1,2 (an ethylene-vinyl acetate copolymer (DuPont Chemical Co. Wilmington, DE)), L-tyrosine co-precipitated with the antigen (available from numerous chemical companies); Montanide (a manide-oleate, ISA Seppic Fairfield, NJ)), AdjuPrime (a carbohydrate polymer), Nitrocellulose-absorbed protein, Gerbu adjuvant (C-C Biotech, Poway, CA), and the like.

In some embodiments the compositions of the invention comprise an “effective amount” of a RSV F polypeptide, protein, and/or protein complex of the invention. An “effective amount” is an amount required to achieve a desired end result. Examples of desired end results include, but are not limited to, the generation of a humoral immune response, the generation of a neutralizing antibody response, the generation of a broadly neutralizing antibody response, and the generation of protective immunity. The amount of a RSV F polypeptide, protein, and/or protein complex of the invention that is effective to achieve the desired end result will depend on variety of factors including, but not limited to, the type, subtype, and strain of the RSV virus against which protection or some other therapeutic effect is sought, the species of the intended subject (e.g. whether a human or some other animal species), the age and/or sex of the intended subject, the planned route of administration, the planned dosing regimen, the seriousness of any ongoing influenza infection (e.g. in the case of therapeutic uses), and the like. The effective amount—which may be a range of effective amounts—can be determined by standard techniques without any undue experimentation, for example using in vitro assays and/or in vivo assays in the intended subject species or any suitable animal model species. Suitable assays include, but are not limited to, those that involve extrapolation from dose-response curves and/or other data derived from in vitro and/or in vivo model systems. In some embodiments the effective amount may be determined according to the judgment of a medical or veterinary practitioner based on the specific circumstances.

### Uses of the RSV F Polypeptides, Proteins & Protein Complexes of the Invention

In some embodiments, the RSV F polypeptides, proteins, and protein complexes of the invention may be useful as research tools, as diagnostic tools, as therapeutic agents, as targets for the production of antibody reagents or therapeutic antibodies, and/or as vaccines or components of vaccine compositions. For example, in some embodiments the RSV F polypeptides, proteins, and protein complexes of the invention are useful as a vaccine immunogens in animal subjects, such as mammalian subject, including humans. These and other uses of the RSV F polypeptides, proteins, and protein complexes of the invention are described more fully below. Those of skill in the art will appreciate that the RSV F polypeptides, proteins, and protein complexes of the invention may be useful for a variety of other applications also, and all such applications and uses are intended to fall within the scope of this invention.

### Tools for Studying RSV F Antibodies

In one embodiment, the RSV F polypeptides, proteins, and protein complexes of the invention may be useful as analytes for assaying and/or measuring binding of, and/or titers of, anti-RSV F antibodies, for example in ELISA assays, Biacore/SPR binding assays, and/or any other assays for antibody binding known in the art. For example, the RSV F polypeptides, proteins, and protein complexes of the invention could be used to analyze, and/or compare the efficacy of anti-RSV F antibodies.

### Tools for Generation of Antibodies

The RSV F polypeptides, proteins, and protein complexes of the invention may also be useful for the generation of therapeutic antibodies and/or antibodies that can be used as research tools or for any other desired use. For example, the RSV F polypeptides, proteins, and protein complexes of the invention can be used for immunizations to obtain antibodies to the RSV F protein for use as research tools and/or as therapeutics. In some embodiments the RSV F polypeptides, proteins, and protein complexes of the invention can be used to immunize a non-human animal, such as a vertebrate, including, but not limited to, a mouse, rat, guinea pig, rabbit, goat, non-human primate, etc. in order to generate antibodies. Such antibodies, which may be monoclonal or polyclonal, and/or cells that produce such antibodies, can then be obtained from the animal. For example, in some embodiments RSV F polypeptides, proteins, and protein complexes of the invention may be used to immunize a mouse and to produce and obtain monoclonal antibodies, and/or hybridomas that produce such monoclonal antibodies. Such methods can be carried out using standard methods known in the art for the production of mouse monoclonal antibodies, including standard methods for hybridoma production. In some embodiments RSV F polypeptides, proteins, and protein complexes of the invention may be used for the production of a chimeric (e.g. part-human), humanized, or fully-human antibody, for example using any of the methods currently known in the art for production of chimeric, humanized and fully human antibodies, including, but not limited to, CDR grafting methods, phage-display methods, transgenic mouse methods (e.g. using a mouse that has been genetically altered to allow for the production of fully human antibodies, such as the Xenomouse) and/or any other suitable method known in the art. Antibodies to the RSV F polypeptides, proteins, and protein complexes of the invention made using such systems can be characterized antigenically using one or a set of several antigens, preferably including the RSV F polypeptides, proteins, and protein complexes of the invention themselves. Additional characterization of such antibodies may be carried out by any standard methods known to one of ordinary skill in the art, including, but not limited to, ELISA-based methods, SPR-based methods, biochemical methods (such as, but not limited to, iso-electric point determination), and methods known in the art for studying biodistribution, safety, and efficacy of antibodies—for example in preclinical and clinical studies.

### Administration to Subjects

In some embodiments, the present invention provides methods that comprise administering the RSV F polypeptides, proteins and/or protein complexes of the invention (or compositions comprising such RSV F polypeptides, proteins and/or protein complexes) to subjects. Such methods may comprise methods for treating individuals having RSV (i.e. therapeutic methods) and/or methods for protecting individuals against future RSV infection (i.e. prophylactic methods).

Subjects to which the RSV F polypeptides, proteins and/or protein complexes of the invention, or compositions comprising such RSV F polypeptides, proteins and/or protein complexes, can be administered (for example in the course of a method of treatment or a method of vaccination) include any and all animal species, including, in particular, those that are susceptible to RSV infection or that can provide model animal systems for the study of RSV infection. In some embodiments, the subjects are mammalian species. In some embodiments, the subjects are avian species. Mammalian subjects include, but are not limited to, humans, non-human primates, rodents, rabbits, and ferrets. Avian subjects include, but are not limited to chickens, such as those on poultry farms. In some embodiments the subjects to which the RSV F polypeptides, proteins and/or protein complexes of the invention, or compositions comprising such RSV F polypeptides, proteins and/or protein complexes are administered, either have RSV, or are at risk of RSV infection. In some embodiments, the subjects are immuno-compromised. In some embodiments, the subjects have a heart disease or disorder. In some embodiments, the subject is a human of greater than about 50 years in age, greater than about 55 years in age, greater than about 60 years in age, greater than about 65 years in age, greater than about 70 years in age, greater than about 75 years in age, greater than about 80 years in age, greater than about 85 years in age, or greater than about 90 years in age. In some embodiments, the subject is a human of less than about 1 month in age, less than about 2 months in age, less than about 3 months in age, less than about 4 months in age, less than about 5 months in age, less than about 6 months in age, less than about 7 months in age, less than about 8 months in age, less than about 9 months in age, less than about 10 months in age, less than about 11 months in age, less than about 12 months in age, less than about 13 months in age, less than about 14 months in age, less than about 15 months in age, less than about 16 months in age, less than about 17 months in age, less than about 18 months in age, less than about 19 months in age, less than about 20 months in age, less than about 21 months in age, less than about 22 months in age, less than about 23 months in age, or less than about 24 months in age.

Various delivery systems are known in the art and any suitable delivery systems can be used to administer the compositions of the present invention to subjects. Such delivery systems include, but are not limited to, intradermal, intramuscular, intraperitoneal, intravenous, subcutaneous, intranasal, epidural, and oral delivery systems. The compositions of the present invention may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. Pulmonary administration can also be employed, e.g., by use of an inhaler or nebulizer, and formulation with an aerosolizing agent.

In some embodiments it may be desirable to administer the pharmaceutical compositions of the invention locally to a tissue in which the RSV F protein or polypeptide may be most effective in generating a desirable outcome. This may be achieved by, for example, local infusion, injection, delivery using a catheter, or by means of an implant, such as a porous, non-porous, or gelatinous implant or an implant comprising one or more membranes (such as sialastic membranes) or fibers from or through which the protein or protein complexes may be released locally. In some embodiments a controlled release system may be used. In some embodiments a pump may be used (see Langer, supra; Sefton, 1987. CRC Crit. Ref. Biomed. Eng. 14:201; Buchwald et al., 1980. Surgery 88:507; Saudek et al., 1989. N. Engl. J. Med. 321:574). In some embodiments polymeric materials may be used to facilitate and/or control release of the RSV prefusion F protein of the invention (see Medical Applications of Controlled Release, Langer and Wise (eds.), 1974. CRC Pres., Boca Raton, Florida; Controlled Drug Bioavailability, 1984. Drug Product Design and Performance, Smolen and Ball (eds.), Wiley, New York; Ranger & Peppas, 1983 Macromol. Sci. Rev. Macromol. Chem. 23:61; see also Levy et al., 1985. Science 228:190; During et al, 1989. Ann. Neurol. 25:351; Howard et al., 1989. J. Neurosurg 71:105). In some embodiments a controlled release system can be placed in proximity to the tissue/organ to which the RSV prefusion F protein or polypeptide is to be delivered (see, e.g., Goodson, 1984. Medical Applications of Controlled Release, supra, vol. 2:115-138). Some suitable controlled release systems that may be used in conjunction with the present invention are described Langer, 1990, Science; vol. 249: pp. 527-1533

In some embodiments, administration of the compositions of the invention can be performed in conjunction with administration of one or more immunostimulatory agents. Non-limiting examples of such immunostimulatory agents include various cytokines, lymphokines and chemokines with immunostimulatory, immunopotentiating, and pro-inflammatory activities, such as interleukins (e.g., IL-1, IL-2, IL-3, IL-4, IL-12, IL-13); growth factors (e.g., granulocyte-macrophage (GM)-colony stimulating factor (CSF)); and other immunostimulatory agents, such as macrophage inflammatory factor, Flt3 ligand, B7.1; B7.2. The immunostimulatory agents can be administered in the same formulation as the RSV F protein or polypeptide, or can be administered separately.

In some embodiments, the RSV F polypeptides, proteins, and/or protein complexes of the invention, or compositions comprising them, can be administered to subjects in a variety of different RSV vaccination methods or regimens. In some such embodiments, administration of a single dose is preferred. However, in other embodiments, additional dosages can be administered, by the same or different route, to achieve the desired prophylactic effect. In neonates and infants, for example, multiple administrations may be required to elicit sufficient levels of immunity. Administration can continue at intervals throughout childhood, as necessary to maintain sufficient levels of protection against RSV infection. Similarly, adults who are particularly susceptible to RSV infection, such as, for example, the elderly and immunocompromised individuals, may require multiple immunizations to establish and/or maintain protective immune responses. Levels of induced immunity can be monitored, for example, by measuring amounts of neutralizing secretory and serum antibodies, and dosages adjusted or vaccinations repeated as necessary to elicit and maintain desired levels of protection.

In some embodiments, dosing regimens may comprise a single administration/immunization. In other embodiments, dosing regimens may comprise multiple administrations/immunizations. For example, vaccines may be given as a primary immunization followed by one or more boosters. In some embodiments of the present invention such a “prime-boost” vaccination regimen may be used. For example, in some such prime-boost regimens a composition comprising a RSV F polypeptide, protein or protein complex as described herein may be administered to an individual on multiple occasions (such as two, three, or even more occasions) separated in time, with the first administration being the “priming” administration and subsequent administrations being “booster” administrations. In other such prime-boost regimens a composition comprising a RSV F polypeptide, protein or protein complex as described herein may be administered to an individual after first administering to the individual a composition comprising a viral or DNA vector encoding an RSV polypeptide, protein or protein complex as a “priming” administration, with one or more subsequent “booster” administrations of a composition comprising a RSV F polypeptide, protein or protein complex as described herein. Boosters may be delivered via the same and/or different route as the primary immunization. Boosters are generally administered after a time period after the primary immunization or the previously administered booster. For example, a booster can be given about two weeks or more after a primary immunization, and/or a second booster can be given about two weeks or more after the first boosters. Boosters may be given repeatedly at time periods, for example, about two weeks or greater throughout up through the entirety of a subject's life. Boosters may be spaced, for example, about two weeks, about three weeks, about four weeks, about one month, about two months, about three months, about four months, about five months, about six months, about seven months, about eight months, about nine months, about ten months, about eleven months, about one year, about one and a half years, about two years, about two and a half years, about three years, about three and a half years, about four years, about four and a half years, about five years, or more after a primary immunization or after a previous booster.

Preferred unit dosage formulations are those containing a dose or unit (e.g. an effective amount), or an appropriate fraction thereof, of the RSV F polypeptides, proteins, and/or protein complexes of the invention. In addition to such ingredients, formulations of the present invention may include other agents commonly used by one of ordinary skill in the art. Pharmaceutical compositions provided by the invention may be conveniently presented in preferred unit dosage formulations prepared using conventional pharmaceutical techniques. Such techniques include the step of bringing into association the active ingredient and the pharmaceutical carrier(s) or excipient(s) or other ingredients. In general, the formulations are prepared by uniformly and intimately bringing into association the active ingredient with liquid carriers. Formulations suitable for parenteral administration include aqueous and non-aqueous sterile injection solutions which may contain anti-oxidants, buffers, bacteriostats and solutes which render the formulation isotonic with the blood of the intended recipient, and aqueous and non-aqueous sterile suspensions which may include suspending agents and thickening agents. The formulations may be presented in unit-dose or multi-dose containers, for example, sealed ampoules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets commonly used by one of ordinary skill in the art.

### Kits

The present invention further provides kits comprising RSV polypeptides, proteins or protein complexes of the invention, or compositions containing such polypeptides, proteins or protein complexes. To facilitate use of the methods and compositions of the invention, any of the components and/or compositions described herein, and additional components useful for experimental or therapeutic or vaccine purposes, can be packaged in the form of a kit. Typically, the kit contains, in addition to the above components, additional materials which can include, e.g., instructions for using the components, packaging material, a container, and/or a delivery device.

Various embodiments of the present invention may also be further described by the following non-limiting examples:

### EXAMPLE

RSV F protein variants E222Y (SEQ ID NO:13), K226Y (SEQ ID NO:15), V469Y (SEQ ID NO: 16), N88Y/S255Y (SEQ ID NO:18), V185Y/K427Y (SEQ ID NO:20) were expressed in human cells as modified by the introduction of di-tyrosine bonds as described below.

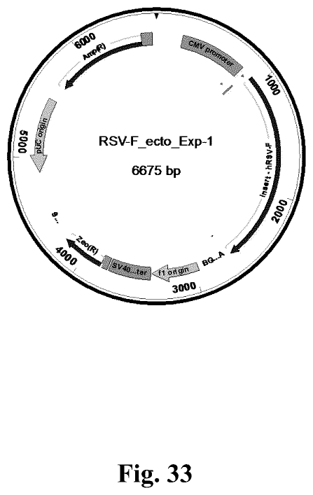

Expression Plasmids. cDNA encoding a C-terminal fusion of the WT human RSV-F ectodomain or DS-Cav1 protein ectodomain to the T4 fibritin foldon trimerization motif, thrombin cleavage-site, 6×HIS-tag (SEQ ID NO: 46), and strep-tag were codon-optimized for human expression and synthesized (Geneart). cDNA encoding RSV F protein variants E222Y (SEQ ID NO:13), K226Y (SEQ ID NO:15), V469Y (SEQ ID NO:16), N88Y/S255Y (SEQ ID NO: 18), V185Y/K427Y (SEQ ID NO:20) were also synthesized. These DNA sequences were cloned into the pCDNA3.1/zeo+expression vector (Invitrogen) via 5′ BamHI and 3′XhoI restriction endonuclease sites using standard methods (FIG. 33).

Cells and Transfections. HEK 293 cells (ATCC) were grown in Dulbecco's Modification of Eagle's Medium (DMEM, Invitrogen) supplemented with 10% Fetal Bovine Serum and 50 μg/ml gentamycin. Cells were seeded into 6-well tissue culture plates (Corning) and grown till 80% confluent (˜24 h). Cells were transfected with 2 μg of each RSV-F expression plasmid per well using a 1:4 ratio (M/V) of DNA to polyethylenimine (25 kDa, linear). 16 h post-transfection, media was removed and replaced with 2 ml/well of serum-free Freestyle-293 expression media (Invitrogen). Cells were cultured at 37 degrees C. for an additional 48 h-72 h in 5% CO₂ prior to collection and analysis.

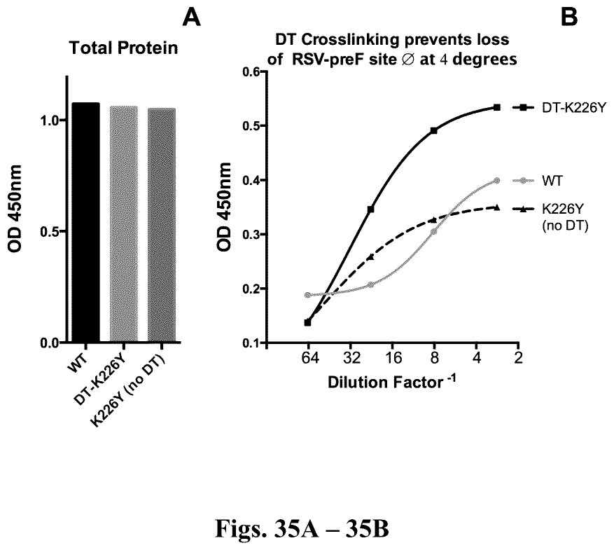

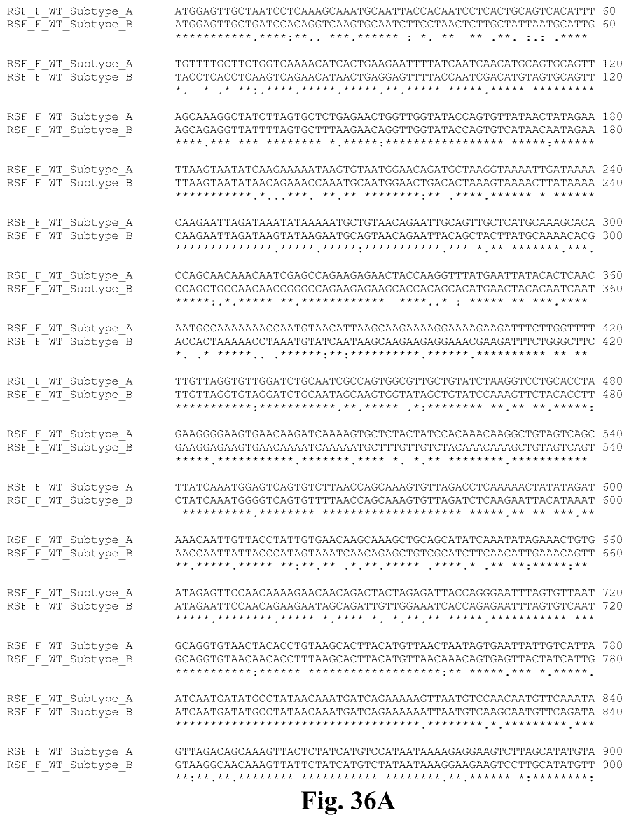

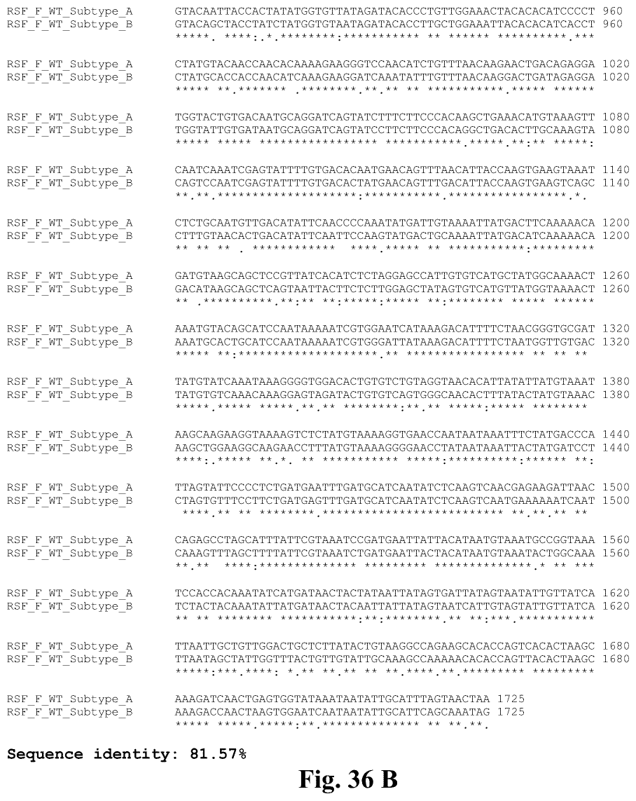

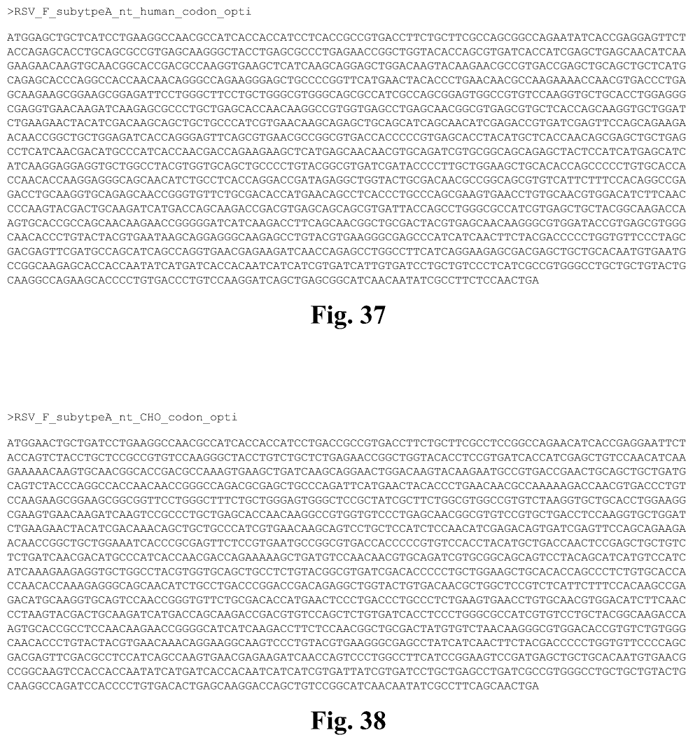

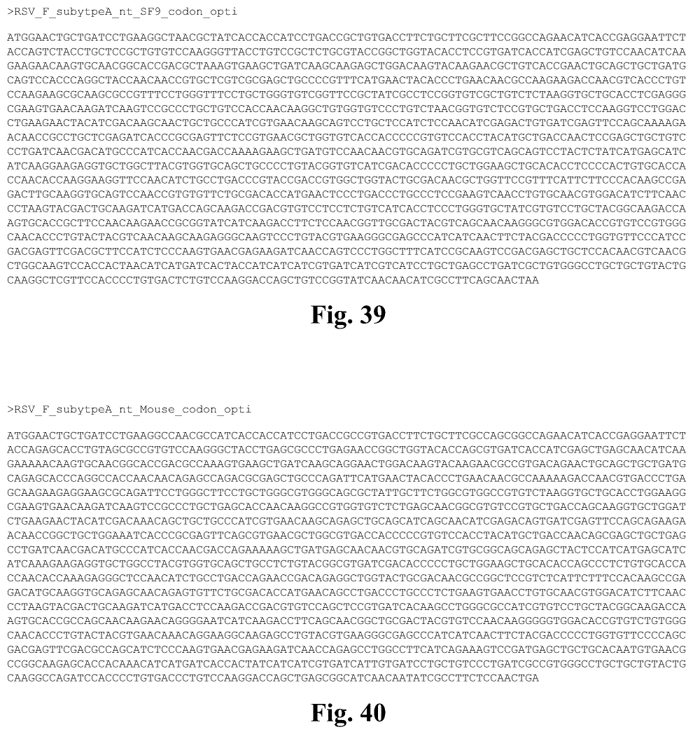

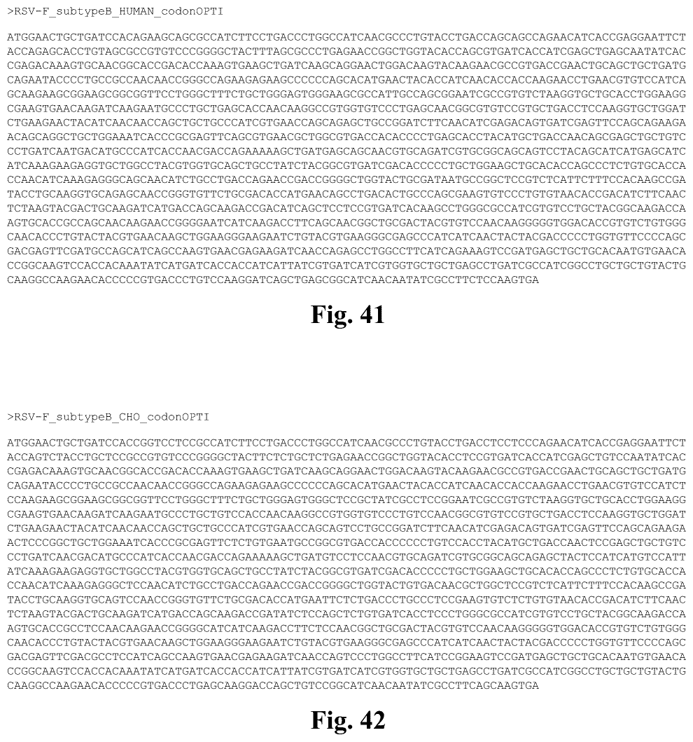

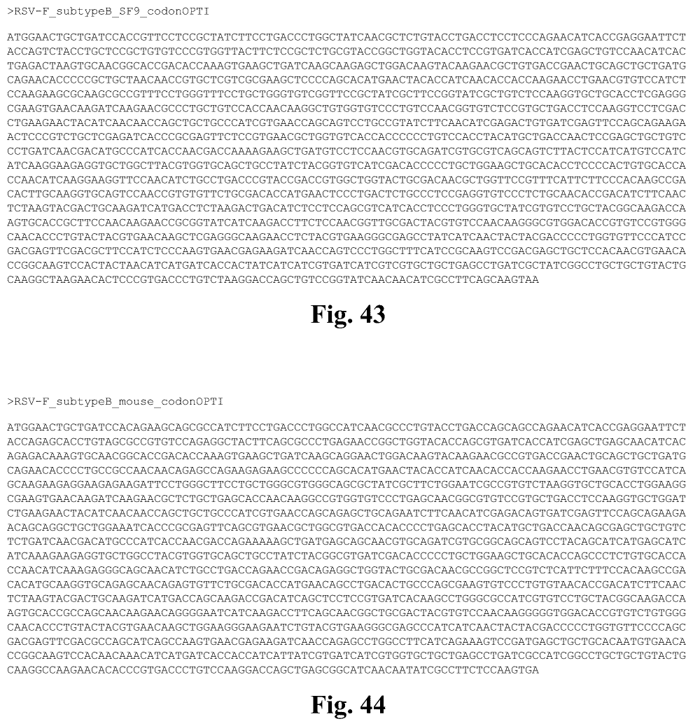

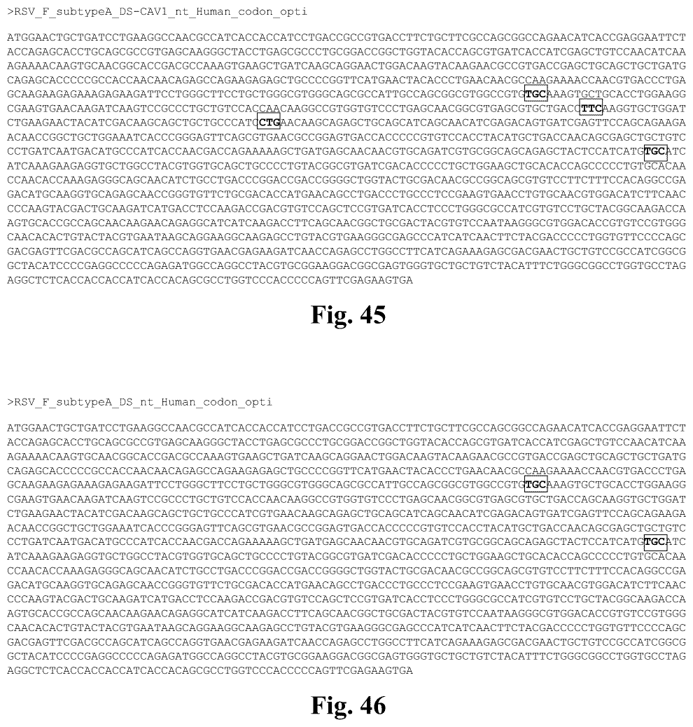

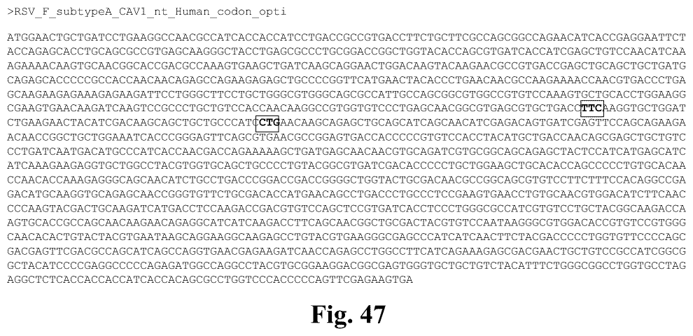

Detection of RSV-F in Cell Supernatants by ELISA. After collection, total RSV-F protein was directly captured from cell supernatants for 1 h at room temperature in EIA/RIA high-bind 96-well plates (Corning). Protein-containing and control wells were subsequently blocked with 4% nonfat milk in PBS-tween20 (0.05%) for 1 h at room temperature. Plates were washed 3× with PBS-T (400 μl/well). Total RSV-F was detected for 1 h using a high-affinity human anti-hRSV antibody (100 ng/ml in PBS) that recognizes both pre- and post-fusion forms of RSV-F. Prefusion F was detected using a pre-F specific human monoclonal antibody (2 μg/ml in PBS) that recognizes site ø. Wells were again washed 3× in PBS-T followed by a 1 h room temperature incubation with an HRP-conjugated goat anti-human F (ab) 2 (Jackson Immunoresearch) at a 1:5000 dilution in PBS. Wells were washed 6× with PBS-T and total RSV-F was detected and quantified using 100 μl 3,3′,5,5′-tetramethylbenzidine (TMB) to produce a colorimetric signal. The colorimetric reaction was stopped by the addition of equal volume 4N sulfuric acid. Final Optical Density readings were taking at 450 nm using a BioRad Benchmark Plus microplate absorbance spectrophotometer. A 2× serial dilution series for each supernatant was used to determine the linear range of detectable signal for each sample allowing accurate comparison of the relative amount of RSV-F between samples (FIG. 35).

Di-tyrosine-Cross-linking in Cell Supernatants. Immediately following collection, 100 μl of transfected and control cell supernatants were transferred to wells of black, flat-bottom, non-binding 96-well FIA plates (Greiner bio-one). 300 ng of Arthromyces ramosus peroxidase was added to each sample to be cross-linked. 1 ul of 1.2 mM H₂O₂ was then added to both control and DT reactions for a final reaction concentration of 120 μM H₂O₂. Reactions were allowed to proceed for 20 minutes at room temperature followed by alkalization of the reactions by addition of equal volume sodium phosphate buffer at pH 10. Di-tyrosine specific fluorescence was read at an excitation wavelength of 320 nm and emission wavelength of 405 nm using a Thermo Scientific Fluoroskan Ascent FL (FIG. 34).

72 h post transfection, supernatents were cross-linked (DT) or left uncross-linked and total protein was measured by ELISA using a high-affinity human anti-hRSV antibody (100 ng/ml in PBS) that recognizes both pre- and post-fusion forms of RSV-F. See FIG. 35A. Following storage at 4 degrees C. for 16 days presentation of site ø was measured by ELISA using a preF specific human monoclonal antibody (2 μg/ml in PBS) that recognizes site ø. Di-tyrosine cross-links were found to stabilize key epitope on RSV prefusion F protein. See FIG. 35B.

While the foregoing invention has been described in some detail for purposes of clarity and understanding, it will be clear to one skilled in the art from a reading of this disclosure that various changes in form and detail can be made without departing from the true scope of the invention. The invention may also be further defined in terms of the following claims.

1. A RSV F polypeptide, protein or protein complex comprising an amino acid sequence having at least 70% sequence identity to amino acid residues 1-513 of SEQ ID NO. 1 (RSV type A) or amino acid residues 1-513 of SEQ ID NO. 3 (RSV type B), wherein the amino acid sequence comprises a point mutation to tyrosine at one or more of amino acid positions 77, 88, 97, 147, 150, 155, 159, 183, 185, 187, 220, 222, 223, 226, 255, 427 or 469.

2. A RSV F polypeptide, protein or protein complex according to claim 1, wherein the polypeptide, protein or protein complex is folded into the pre-F conformation and comprises at least one di-tyrosine cross-link, wherein one or both tyrosines of the at least one cross-link originate from a point mutation to tyrosine, and wherein the cross-links are located between one or more paired tyrosine amino acid residues located at amino acid positions: 147 and 286; 198 and 220; 198 and 222; 198 and 223; 198 and 226; 33 and 469; 77 and 222; 88 and 255; 97 and 159; 183 and 427; 185 and 427; 88 and 255; or 187 and 427.

3. A RSV F polypeptide, protein or protein complex comprising according to claim 1, comprising amino acid residues 1-513 of sequence of SEQ ID NO. 11, 12, 13, 14, 15, 16, 17, 18, 19, 20, 21, 22, 29, 30, 31 or 32.

4. A RSV F polypeptide, protein or protein complex according to claim 1, wherein the polypeptide, protein or protein complex is capable of folding into the pre-F conformation.

5. A RSV F polypeptide, protein or protein complex according to claim 1, wherein the polypeptide, protein or protein complex further comprises one or more point mutations to cysteine.

6. A RSV F polypeptide, protein or protein complex according to claim 1, wherein the polypeptide, protein or protein complex further comprises one or more cavity-filling hydrophobic amino acid substitutions.

7. A RSV F polypeptide, protein or protein complex according to claim 1, wherein the polypeptide, protein or protein complex further comprises a trimerization domain.

8. A RSV F polypeptide, protein or protein complex according to claim 7, wherein the trimerization domain is a foldon domain.

9. A RSV F polypeptide, protein or protein complex according to claim 1, wherein the polypeptide, protein or protein complex is capable of eliciting production of RSV-specific antibodies in a subject.

10. A RSV F polypeptide, protein or protein complex according to claim 1, wherein the polypeptide, protein or protein complex is capable of binding to an antibody that recognizes antigenic site ø.

11. A nucleic acid molecule encoding a RSV F polypeptide, protein or protein complex according to claim 1.

12. A composition comprising a RSV F polypeptide, protein or protein complex according to claim 1.

13. The composition of claim 12, wherein the composition is a vaccine composition.

14. The composition of claim 13, wherein the composition further comprises an adjuvant, a carrier, an immunostimulatory agent, or any combination thereof.

15. A method of vaccinating a subject against RSV, the method comprising administering to a subject a composition comprising an effective amount of a RSV F polypeptide, protein or protein complex according to claim 1.

16. The method of claim 15, wherein the subject is a human of less than 24 months in age.

17. The method of claim 15, wherein the subject is a human of greater than 50 years in age.

18. The method of claim 15, wherein the administering comprises administering a single dosage of the composition.

19. The method of claim 15, wherein the administering comprises administering a first “priming” dosage of the composition and one or more subsequent “boosting” dosages of the composition.


SEQUENCE LISTING (47 sequences)

SEQ ID NO: 1 - protein, 568 aa, Human respiratory syncytial virus
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFLGGLVPRGSHHHHHHSAWSHPQFEK

SEQ ID NO: 2 - protein, 574 aa, Human respiratory syncytial virus
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNTGKSTTNIMITTIIIVIIVVLLSLIAIGLLLYCKAKNTPVTLSKDQLSGINNIAFSK

SEQ ID NO: 3 - protein, 568 aa, Human respiratory syncytial virus
MELLIHRSSAIFLTLAINALYLTSSQNITEEFYQSTCSAVSRGYFSALRTGWYTSVITIELSNITETKCNGTDTKVKLIKQELDKYKNAVTELQLLMQNTPAANNRARREAPQHMNYTINTTKNLNVSISKKRKRRFLGFLLGVGSAIASGIAVSKVLHLEGEVNKIKNALLSTNKAVVSLSNGVSVLTSKVLDLKNYINNQLLPIVNQQSCRIFNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRKSDELLSAIGGYIPEAPRDGQAYVRKDGEWVLLSTFLGGLVPRGSHHHHHHSAWSHPQFEK

SEQ ID NO: 4 - protein, 574 aa, Human respiratory syncytial virus
MELLIHRSSAIFLTLAINALYLTSSQNITEEFYQSTCSAVSRGYFSALRTGWYTSVITIELSNITETKCNGTDTKVKLIKQELDKYKNAVTELQLLMQNTPAANNRARREAPQHMNYTINTTKNLNVSISKKRKRRFLGFLLGVGSAIASGIAVSKVLHLEGEVNKIKNALLSTNKAVVSLSNGVSVLTSKVLDLKNYINNQLLPIVNQQSCRIFNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRKSDELLHNVNTGKSTTNIMITTIIIVIIVVLLSLIAIGLLLYCKAKNTPVTLSKDQLSGINNIAFSK

SEQ ID NO: 5 - protein, 568 aa, synthetic construct
  REGION 1..568: note = Description of Artificial Sequence: Synthetic polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFLGGLVPRGSHHHHHHSAWSHPQFEK

SEQ ID NO: 6 - protein, 574 aa, synthetic construct
  REGION 1..574: note = Description of Artificial Sequence: Synthetic polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNTGKSTTNIMITTIIIVIIVVLLSLIAIGLLLYCKAKNTPVTLSKDQLSGINNIAFSK

SEQ ID NO: 7 - protein, 568 aa, synthetic construct
  REGION 1..568: note = Description of Artificial Sequence: Synthetic polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFLGGLVPRGSHHHHHHSAWSHPQFEK

SEQ ID NO: 8 - protein, 574 aa, synthetic construct
  REGION 1..574: note = Description of Artificial Sequence: Synthetic polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNTGKSTTNIMITTIIIVIIVVLLSLIAIGLLLYCKAKNTPVTLSKDQLSGINNIAFSK

SEQ ID NO: 9 - protein, 568 aa, synthetic construct
  REGION 1..568: note = Description of Artificial Sequence: Synthetic polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFLGGLVPRGSHHHHHHSAWSHPQFEK

SEQ ID NO: 10 - protein, 574 aa, synthetic construct
  REGION 1..574: note = Description of Artificial Sequence: Synthetic polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNTGKSTTNIMITTIIIVIIVVLLSLIAIGLLLYCKAKNTPVTLSKDQLSGINNIAFSK

SEQ ID NO: 11 - protein, 568 aa, synthetic construct
  REGION 1..568: note = Description of Artificial Sequence: Synthetic polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSYIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFLGGLVPRGSHHHHHHSAWSHPQFEK

SEQ ID NO: 12 - protein, 568 aa, synthetic construct
  REGION 1..568: note = Description of Artificial Sequence: Synthetic polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETYIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFLGGLVPRGSHHHHHHSAWSHPQFEK

SEQ ID NO: 13 - protein, 568 aa, synthetic construct
  REGION 1..568: note = Description of Artificial Sequence: Synthetic polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIYFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFLGGLVPRGSHHHHHHSAWSHPQFEK

SEQ ID NO: 14 - protein, 568 aa, synthetic construct
  REGION 1..568: note = Description of Artificial Sequence: Synthetic polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEYQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFLGGLVPRGSHHHHHHSAWSHPQFEK

SEQ ID NO: 15 - protein, 568 aa, synthetic construct
  REGION 1..568: note = Description of Artificial Sequence: Synthetic polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQYNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFLGGLVPRGSHHHHHHSAWSHPQFEK

SEQ ID NO: 16 - protein, 568 aa, synthetic construct
  REGION 1..568: note = Description of Artificial Sequence: Synthetic polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYYKGEPIINFYDPLVFPSDEFDASISQVNEKINQSLAFIRKSDELLSAIGGYIPEAPRDGQAYVRKDGEWVLLSTFLGGLVPRGSHHHHHHSAWSHPQFEK

SEQ ID NO: 17 - protein, 568 aa, synthetic construct
  REGION 1..568: note = Description of Artificial Sequence: Synthetic polypeptide
MELLILKANAITTILTAVTFCFASGQNITEEFYQSTCSAVSKGYLSALRTGWYTSVITIELSNIKENKCNGTDAKVYLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIYFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFLGGLVPRGSHHHHHHSAWSHPQFEK

SEQ ID NO: 18 - protein, 568 aa, synthetic construct
  REGION 1..568: note = Description of Artificial Sequence: Synthetic polypeptide
MELLILKANAITTILTAVTFCFASGQNITEEFYQSTCSAVSKGYLSALRTGWYTSVITIELSNIKENKCNGTDAKVKLIKQELDKYKYAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNYELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFLGGLVPRGSHHHHHHSAWSHPQFEK

SEQ ID NO: 19 - protein, 568 aa, synthetic construct
  REGION 1..568: note = Description of Artificial Sequence: Synthetic polypeptide
MELLILKANAITTILTAVTFCFASGQNITEEFYQSTCSAVSKGYLSALRTGWYTSVITIELSNIKENKCNGTDAKVKLIKQELDKYKNAVTELQLLYQSTPATNNRARRELPRFMNYTLNNAKKTNVTLSKKRKRRFLGFLLGVGSAIASGVAVSKVLYLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFLGGLVPRGSHHHHHHSAWSHPQFEK

SEQ ID NO: 20 - protein, 568 aa, synthetic construct
  REGION 1..568: note = Description of Artificial Sequence: Synthetic polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGYSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNYNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFLGGLVPRGSHHHHHHSAWSHPQFEK

SEQ ID NO: 21 - protein, 568 aa, synthetic construct
  REGION 1..568: note = Description of Artificial Sequence: Synthetic polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSYLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNYNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFLGGLVPRGSHHHHHHSAWSHPQFEK

SEQ ID NO: 22 - protein, 568 aa, synthetic construct
  REGION 1..568: note = Description of Artificial Sequence: Synthetic polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSYGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNYNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFLGGLVPRGSHHHHHHSAWSHPQFEK

SEQ ID NO: 23 - protein, 568 aa, synthetic construct
  REGION 1..568: note = Description of Artificial Sequence: Synthetic polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSYIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFLGGLVPRGSHHHHHHSAWSHPQFEK

SEQ ID NO: 24 - protein, 568 aa, synthetic construct
  REGION 1..568: note = Description of Artificial Sequence: Synthetic polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETYIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFLGGLVPRGSHHHHHHSAWSHPQFEK

SEQ ID NO: 25 - protein, 568 aa, synthetic construct
  REGION 1..568: note = Description of Artificial Sequence: Synthetic polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIYFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFLGGLVPRGSHHHHHHSAWSHPQFEK

SEQ ID NO: 26 - protein, 568 aa, synthetic construct
  REGION 1..568: note = Description of Artificial Sequence: Synthetic polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEYQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFLGGLVPRGSHHHHHHSAWSHPQFEK

SEQ ID NO: 27 - protein, 568 aa, synthetic construct
  REGION 1..568: note = Description of Artificial Sequence: Synthetic polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQYNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFLGGLVPRGSHHHHHHSAWSHPQFEK

SEQ ID NO: 28 - protein, 568 aa, synthetic construct
  REGION 1..568: note = Description of Artificial Sequence: Synthetic polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYYKGEPIINFYDPLVFPSDEFDASISQVNEKINQSLAFIRKSDELLSAIGGYIPEAPRDGQAYVRKDGEWVLLSTFLGGLVPRGSHHHHHHSAWSHPQFEK

SEQ ID NO: 29 - protein, 568 aa, synthetic construct
  REGION 1..568: note = Description of Artificial Sequence: Synthetic polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIYFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYYKGEPIINFYDPLVFPSDEFDASISQVNEKINQSLAFIRKSDELLSAIGGYIPEAPRDGQAYVRKDGEWVLLSTFLGGLVPRGSHHHHHHSAWSHPQFEK

SEQ ID NO: 30 - protein, 568 aa, synthetic construct
  REGION 1..568: note = Description of Artificial Sequence: Synthetic polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQYNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYYKGEPIINFYDPLVFPSDEFDASISQVNEKINQSLAFIRKSDELLSAIGGYIPEAPRDGQAYVRKDGEWVLLSTFLGGLVPRGSHHHHHHSAWSHPQFEK

SEQ ID NO: 31 - protein, 568 aa, synthetic construct
  REGION 1..568: note = Description of Artificial Sequence: Synthetic polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIYFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYYKGEPIINFYDPLVFPSDEFDASISQVNEKINQSLAFIRKSDELLSAIGGYIPEAPRDGQAYVRKDGEWVLLSTFLGGLVPRGSHHHHHHSAWSHPQFEK

SEQ ID NO: 32 - protein, 568 aa, synthetic construct
  REGION 1..568: note = Description of Artificial Sequence: Synthetic polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQYNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYYKGEPIINFYDPLVFPSDEFDASISQVNEKINQSLAFIRKSDELLSAIGGYIPEAPRDGQAYVRKDGEWVLLSTFLGGLVPRGSHHHHHHSAWSHPQFEK

SEQ ID NO: 33 - DNA, 1725 bp, Human respiratory syncytial virus
  source 1..1725: mol_type = unassigned DNA
atggagttgctaatcctcaaagcaaatgcaattaccacaatcctcactgcagtcacattttgttttgcttctggtcaaaacatcactgaagaattttatcaatcaacatgcagtgcagttagcaaaggctatcttagtgctctgagaactggttggtataccagtgttataactatagaattaagtaatatcaagaaaaataagtgtaatggaacagatgctaaggtaaaattgataaaacaagaattagataaatataaaaatgctgtaacagaattgcagttgctcatgcaaagcacaccagcaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatcgccagtggcgttgctgtatctaaggtcctgcacctagaaggggaagtgaacaagatcaaaagtgctctactatccacaaacaaggctgtagtcagcttatcaaatggagtcagtgtcttaaccagcaaagtgttagacctcaaaaactatatagataaacaattgttacctattgtgaacaagcaaagctgcagcatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaagtaaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaaggggtggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgccggtaaatccaccacaaatatcatgataactactataattatagtgattatagtaatattgttatcattaattgctgttggactgctcttatactgtaaggccagaagcacaccagtcacactaagcaaagatcaactgagtggtataaataatattgcatttagtaactaa

SEQ ID NO: 34 - DNA, 1725 bp, Human respiratory syncytial virus
  source 1..1725: mol_type = unassigned DNA
atggagttgctgatccacaggtcaagtgcaatcttcctaactcttgctattaatgcattgtacctcacctcaagtcagaacataactgaggagttttaccaatcgacatgtagtgcagttagcagaggttattttagtgctttaagaacaggttggtataccagtgtcataacaatagaattaagtaatataacagaaaccaaatgcaatggaactgacactaaagtaaaacttataaaacaagaattagataagtataagaatgcagtaacagaattacagctacttatgcaaaacacgccagctgccaacaaccgggccagaagagaagcaccacagcacatgaactacacaatcaataccactaaaaacctaaatgtatcaataagcaagaagaggaaacgaagatttctgggcttcttgttaggtgtaggatctgcaatagcaagtggtatagctgtatccaaagttctacaccttgaaggagaagtgaacaaaatcaaaaatgctttgttgtctacaaacaaagctgtagtcagtctatcaaatggggtcagtgttttaaccagcaaagtgttagatctcaagaattacataaataaccaattattacccatagtaaatcaacagagctgtcgcatcttcaacattgaaacagttatagaattccaacagaagaatagcagattgttggaaatcaccagagaatttagtgtcaatgcaggtgtaacaacacctttaagcacttacatgttaacaaacagtgagttactatcattgatcaatgatatgcctataacaaatgatcagaaaaaattaatgtcaagcaatgttcagatagtaaggcaacaaagttattctatcatgtctataataaaggaagaagtccttgcatatgttgtacagctacctatctatggtgtaatagatacaccttgctggaaattacacacatcacctctatgcaccaccaacatcaaagaaggatcaaatatttgtttaacaaggactgatagaggatggtattgtgataatgcaggatcagtatccttcttcccacaggctgacacttgcaaagtacagtccaatcgagtattttgtgacactatgaacagtttgacattaccaagtgaagtcagcctttgtaacactgacatattcaattccaagtatgactgcaaaattatgacatcaaaaacagacataagcagctcagtaattacttctcttggagctatagtgtcatgttatggtaaaactaaatgcactgcatccaataaaaatcgtgggattataaagacattttctaatggttgtgactatgtgtcaaacaaaggagtagatactgtgtcagtgggcaacactttatactatgtaaacaagctggaaggcaagaacctttatgtaaaaggggaacctataataaattactatgatcctctagtgtttccttctgatgagtttgatgcatcaatatctcaagtcaatgaaaaaatcaatcaaagtttagcttttattcgtaaatctgatgaattactacataatgtaaatactggcaaatctactacaaatattatgataactacaattattatagtaatcattgtagtattgttatcattaatagctattggtttactgttgtattgcaaagccaaaaacacaccagttacactaagcaaagaccaactaagtggaatcaataatattgcattcagcaaatag

SEQ ID NO: 35 - DNA, 1725 bp, synthetic construct
  misc_feature 1..1725: note = Description of Artificial Sequence: Synthetic polynucleotide
  source 1..1725: mol_type = other DNA
atggagctgctcatcctgaaggccaacgccatcaccaccatcctcaccgccgtgaccttctgcttcgccagcggccagaatatcaccgaggagttctaccagagcacctgcagcgccgtgagcaagggctacctgagcgccctgagaaccggctggtacaccagcgtgatcaccatcgagctgagcaacatcaagaagaacaagtgcaacggcaccgacgccaaggtgaagctcatcaagcaggagctggacaagtacaagaacgccgtgaccgagctgcagctgctcatgcagagcacccaggccaccaacaacagggccagaagggagctgccccggttcatgaactacaccctgaacaacgccaagaaaaccaacgtgaccctgagcaagaagcggaagcggagattcctgggcttcctgctgggcgtgggcagcgccatcgccagcggagtggccgtgtccaaggtgctgcacctggagggcgaggtgaacaagatcaagagcgccctgctgagcaccaacaaggccgtggtgagcctgagcaacggcgtgagcgtgctcaccagcaaggtgctggatctgaagaactacatcgacaagcagctgctgcccatcgtgaacaagcagagctgcagcatcagcaacatcgagaccgtgatcgagttccagcagaagaacaaccggctgctggagatcaccagggagttcagcgtgaacgccggcgtgaccacccccgtgagcacctacatgctcaccaacagcgagctgctgagcctcatcaacgacatgcccatcaccaacgaccagaagaagctcatgagcaacaacgtgcagatcgtgcggcagcagagctactccatcatgagcatcatcaaggaggaggtgctggcctacgtggtgcagctgcccctgtacggcgtgatcgataccccttgctggaagctgcacaccagccccctgtgcaccaccaacaccaaggagggcagcaacatctgcctcaccaggaccgatagaggctggtactgcgacaacgccggcagcgtgtcattctttccacaggccgagacctgcaaggtgcagagcaaccgggtgttctgcgacaccatgaacagcctcaccctgcccagcgaagtgaacctgtgcaacgtggacatcttcaaccccaagtacgactgcaagatcatgaccagcaagaccgacgtgagcagcagcgtgattaccagcctgggcgccatcgtgagctgctacggcaagaccaagtgcaccgccagcaacaagaaccgggggatcatcaagaccttcagcaacggctgcgactacgtgagcaacaagggcgtggataccgtgagcgtgggcaacaccctgtactacgtgaataagcaggagggcaagagcctgtacgtgaagggcgagcccatcatcaacttctacgaccccctggtgttccctagcgacgagttcgatgccagcatcagccaggtgaacgagaagatcaaccagagcctggccttcatcaggaagagcgacgagctgctgcacaatgtgaatgccggcaagagcaccaccaatatcatgatcaccacaatcatcatcgtgatcattgtgatcctgctgtccctcatcgccgtgggcctgctgctgtactgcaaggccagaagcacccctgtgaccctgtccaaggatcagctgagcggcatcaacaatatcgccttctccaactga

SEQ ID NO: 36 - DNA, 1725 bp, synthetic construct
  misc_feature 1..1725: note = Description of Artificial Sequence: Synthetic polynucleotide
  source 1..1725: mol_type = other DNA
atggaactgctgatcctgaaggccaacgccatcaccaccatcctgaccgccgtgaccttctgcttcgcctccggccagaacatcaccgaggaattctaccagtctacctgctccgccgtgtccaagggctacctgtctgctctgagaaccggctggtacacctccgtgatcaccatcgagctgtccaacatcaagaaaaacaagtgcaacggcaccgacgccaaagtgaagctgatcaagcaggaactggacaagtacaagaatgccgtgaccgaactgcagctgctgatgcagtctacccaggccaccaacaaccgggccagacgcgagctgcccagattcatgaactacaccctgaacaacgccaaaaagaccaacgtgaccctgtccaagaagcggaagcggcggttcctgggctttctgctgggagtgggctccgctatcgcttctggcgtggccgtgtctaaggtgctgcacctggaaggcgaagtgaacaagatcaagtccgccctgctgagcaccaacaaggccgtggtgtccctgagcaacggcgtgtccgtgctgacctccaaggtgctggatctgaagaactacatcgacaaacagctgctgcccatcgtgaacaagcagtcctgctccatctccaacatcgagacagtgatcgagttccagcagaagaacaaccggctgctggaaatcacccgcgagttctccgtgaatgccggcgtgaccacccccgtgtccacctacatgctgaccaactccgagctgctgtctctgatcaacgacatgcccatcaccaacgaccagaaaaagctgatgtccaacaacgtgcagatcgtgcggcagcagtcctacagcatcatgtccatcatcaaagaagaggtgctggcctacgtggtgcagctgcctctgtacggcgtgatcgacaccccctgctggaagctgcacaccagccctctgtgcaccaccaacaccaaagagggcagcaacatctgcctgacccggaccgacagaggctggtactgtgacaacgctggctccgtctcattctttccacaagccgagacatgcaaggtgcagtccaaccgggtgttctgcgacaccatgaactccctgaccctgccctctgaagtgaacctgtgcaacgtggacatcttcaaccctaagtacgactgcaagatcatgaccagcaagaccgacgtgtccagctctgtgatcacctccctgggcgccatcgtgtcctgctacggcaagaccaagtgcaccgcctccaacaagaaccggggcatcatcaagaccttctccaacggctgcgactatgtgtctaacaagggcgtggacaccgtgtctgtgggcaacaccctgtactacgtgaacaaacaggaaggcaagtccctgtacgtgaagggcgagcctatcatcaacttctacgaccccctggtgttccccagcgacgagttcgacgcctccatcagccaagtgaacgagaagatcaaccagtccctggccttcatccggaagtccgatgagctgctgcacaatgtgaacgccggcaagtccaccaccaatatcatgatcaccacaatcatcatcgtgattatcgtgatcctgctgagcctgatcgccgtgggcctgctgctgtactgcaaggccagatccacccctgtgacactgagcaaggaccagctgtccggcatcaacaatatcgccttcagcaactga

SEQ ID NO: 37 - DNA, 1725 bp, synthetic construct
  misc_feature 1..1725: note = Description of Artificial Sequence: Synthetic polynucleotide
  source 1..1725: mol_type = other DNA
atggaactgctgatcctgaaggctaacgctatcaccaccatcctgaccgctgtgaccttctgcttcgcttccggccagaacatcaccgaggaattctaccagtctacctgctccgctgtgtccaagggttacctgtccgctctgcgtaccggctggtacacctccgtgatcaccatcgagctgtccaacatcaagaagaacaagtgcaacggcaccgacgctaaagtgaagctgatcaagcaagagctggacaagtacaagaacgctgtcaccgaactgcagctgctgatgcagtccacccaggctaccaacaaccgtgctcgtcgcgagctgccccgtttcatgaactacaccctgaacaacgccaagaagaccaacgtcaccctgtccaagaagcgcaagcgccgtttcctgggtttcctgctgggtgtcggttccgctatcgcctccggtgtcgctgtctctaaggtgctgcacctcgagggcgaagtgaacaagatcaagtccgccctgctgtccaccaacaaggctgtggtgtccctgtctaacggtgtctccgtgctgacctccaaggtcctggacctgaagaactacatcgacaagcaactgctgcccatcgtgaacaagcagtcctgctccatctccaacatcgagactgtgatcgagttccagcaaaagaacaaccgcctgctcgagatcacccgcgagttctccgtgaacgctggtgtcaccacccccgtgtccacctacatgctgaccaactccgagctgctgtccctgatcaacgacatgcccatcaccaacgaccaaaagaagctgatgtccaacaacgtgcagatcgtgcgtcagcagtcctactctatcatgagcatcatcaaggaagaggtgctggcttacgtggtgcagctgcccctgtacggtgtcatcgacaccccctgctggaagctgcacacctccccactgtgcaccaccaacaccaaggaaggttccaacatctgcctgacccgtaccgaccgtggctggtactgcgacaacgctggttccgtttcattcttcccacaagccgagacttgcaaggtgcagtccaaccgtgtgttctgcgacaccatgaactccctgaccctgccctccgaagtcaacctgtgcaacgtggacatcttcaaccctaagtacgactgcaagatcatgaccagcaagaccgacgtgtcctcctctgtcatcacctccctgggtgctatcgtgtcctgctacggcaagaccaagtgcaccgcttccaacaagaaccgcggtatcatcaagaccttctccaacggttgcgactacgtcagcaacaagggcgtggacaccgtgtccgtgggcaacaccctgtactacgtcaacaagcaagagggcaagtccctgtacgtgaagggcgagcccatcatcaacttctacgaccccctggtgttcccatccgacgagttcgacgcttccatctcccaagtgaacgagaagatcaaccagtccctggctttcatccgcaagtccgacgagctgctccacaacgtcaacgctggcaagtccaccactaacatcatgatcactaccatcatcatcgtgatcatcgtcatcctgctgagcctgatcgctgtgggcctgctgctgtactgcaaggctcgttccacccctgtgactctgtccaaggaccagctgtccggtatcaacaacatcgccttcagcaactaa

SEQ ID NO: 38 - DNA, 1725 bp, synthetic construct
  misc_feature 1..1725: note = Description of Artificial Sequence: Synthetic polynucleotide
  source 1..1725: mol_type = other DNA
atggaactgctgatcctgaaggccaacgccatcaccaccatcctgaccgccgtgaccttctgcttcgccagcggccagaacatcaccgaggaattctaccagagcacctgtagcgccgtgtccaagggctacctgagcgccctgagaaccggctggtacaccagcgtgatcaccatcgagctgagcaacatcaagaaaaacaagtgcaacggcaccgacgccaaagtgaagctgatcaagcaggaactggacaagtacaagaacgccgtgacagaactgcagctgctgatgcagagcacccaggccaccaacaacagagccagacgcgagctgcccagattcatgaactacaccctgaacaacgccaaaaagaccaacgtgaccctgagcaagaagaggaagcgcagattcctgggcttcctgctgggcgtgggcagcgctattgcttctggcgtggccgtgtctaaggtgctgcacctggaaggcgaagtgaacaagatcaagtccgccctgctgagcaccaacaaggccgtggtgtctctgagcaacggcgtgtccgtgctgaccagcaaggtgctggatctgaagaactacatcgacaaacagctgctgcccatcgtgaacaagcagagctgcagcatcagcaacatcgagacagtgatcgagttccagcagaagaacaaccggctgctggaaatcacccgcgagttcagcgtgaacgctggcgtgaccacccccgtgtccacctacatgctgaccaacagcgagctgctgagcctgatcaacgacatgcccatcaccaacgaccagaaaaagctgatgagcaacaacgtgcagatcgtgcggcagcagagctactccatcatgagcatcatcaaagaagaggtgctggcctacgtggtgcagctgcctctgtacggcgtgatcgacaccccctgctggaagctgcacaccagccctctgtgcaccaccaacaccaaagagggctccaacatctgcctgaccagaaccgacagaggctggtactgcgacaacgccggctccgtctcattctttccacaagccgagacatgcaaggtgcagagcaacagagtgttctgcgacaccatgaacagcctgaccctgccctctgaagtgaacctgtgcaacgtggacatcttcaaccctaagtacgactgcaagatcatgacctccaagaccgacgtgtccagctccgtgatcacaagcctgggcgccatcgtgtcctgctacggcaagaccaagtgcaccgccagcaacaagaacaggggaatcatcaagaccttcagcaacggctgcgactacgtgtccaacaagggggtggacaccgtgtctgtgggcaacaccctgtactacgtgaacaaacaggaaggcaagagcctgtacgtgaagggcgagcccatcatcaacttctacgaccccctggtgttccccagcgacgagttcgacgccagcatctcccaagtgaacgagaagatcaaccagagcctggccttcatcagaaagtccgatgagctgctgcacaatgtgaacgccggcaagagcaccacaaacatcatgatcaccactatcatcatcgtgatcattgtgatcctgctgtccctgatcgccgtgggcctgctgctgtactgcaaggccagatccacccctgtgaccctgtccaaggaccagctgagcggcatcaacaatatcgccttctccaactga

SEQ ID NO: 39 - DNA, 1725 bp, synthetic construct
  misc_feature 1..1725: note = Description of Artificial Sequence: Synthetic polynucleotide
  source 1..1725: mol_type = other DNA
atggaactgctgatccacagaagcagcgccatcttcctgaccctggccatcaacgccctgtacctgaccagcagccagaacatcaccgaggaattctaccagagcacctgtagcgccgtgtcccggggctactttagcgccctgagaaccggctggtacaccagcgtgatcaccatcgagctgagcaatatcaccgagacaaagtgcaacggcaccgacaccaaagtgaagctgatcaagcaggaactggacaagtacaagaacgccgtgaccgaactgcagctgctgatgcagaatacccctgccgccaacaaccgggccagaagagaagccccccagcacatgaactacaccatcaacaccaccaagaacctgaacgtgtccatcagcaagaagcggaagcggcggttcctgggctttctgctgggagtgggaagcgccattgccagcggaatcgccgtgtctaaggtgctgcacctggaaggcgaagtgaacaagatcaagaatgccctgctgagcaccaacaaggccgtggtgtccctgagcaacggcgtgtccgtgctgacctccaaggtgctggatctgaagaactacatcaacaaccagctgctgcccatcgtgaaccagcagagctgccggatcttcaacatcgagacagtgatcgagttccagcagaagaacagcaggctgctggaaatcacccgcgagttcagcgtgaacgctggcgtgaccacacccctgagcacctacatgctgaccaacagcgagctgctgtccctgatcaatgacatgcccatcaccaacgaccagaaaaagctgatgagcagcaacgtgcagatcgtgcggcagcagtcctacagcatcatgagcatcatcaaagaagaggtgctggcctacgtggtgcagctgcctatctacggcgtgatcgacaccccctgctggaagctgcacaccagccctctgtgcaccaccaacatcaaagagggcagcaacatctgcctgaccagaaccgaccggggctggtactgcgataatgccggctccgtctcattctttccacaagccgatacctgcaaggtgcagagcaaccgggtgttctgcgacaccatgaacagcctgacactgcccagcgaagtgtccctgtgtaacaccgacatcttcaactctaagtacgactgcaagatcatgaccagcaagaccgacatcagctcctccgtgatcacaagcctgggcgccatcgtgtcctgctacggcaagaccaagtgcaccgccagcaacaagaaccggggaatcatcaagaccttcagcaacggctgcgactacgtgtccaacaagggggtggacaccgtgtctgtgggcaacaccctgtactacgtgaacaagctggaagggaagaatctgtacgtgaagggcgagcccatcatcaactactacgaccccctggtgttccccagcgacgagttcgatgccagcatcagccaagtgaacgagaagatcaaccagagcctggccttcatcagaaagtccgatgagctgctgcacaatgtgaacaccggcaagtccaccacaaatatcatgatcaccaccatcattatcgtgatcatcgtggtgctgctgagcctgatcgccatcggcctgctgctgtactgcaaggccaagaacacccccgtgaccctgtccaaggatcagctgagcggcatcaacaatatcgccttctccaagtga

SEQ ID NO: 40 - DNA, 1725 bp, synthetic construct
  misc_feature 1..1725: note = Description of Artificial Sequence: Synthetic polynucleotide
  source 1..1725: mol_type = other DNA
atggaactgctgatccaccggtcctccgccatcttcctgaccctggccatcaacgccctgtacctgacctcctcccagaacatcaccgaggaattctaccagtctacctgctccgccgtgtcccggggctacttctctgctctgagaaccggctggtacacctccgtgatcaccatcgagctgtccaatatcaccgagacaaagtgcaacggcaccgacaccaaagtgaagctgatcaagcaggaactggacaagtacaagaacgccgtgaccgaactgcagctgctgatgcagaatacccctgccgccaacaaccgggccagaagagaagccccccagcacatgaactacaccatcaacaccaccaagaacctgaacgtgtccatctccaagaagcggaagcggcggttcctgggctttctgctgggagtgggctccgctatcgcctccggaatcgccgtgtctaaggtgctgcacctggaaggcgaagtgaacaagatcaagaatgccctgctgtccaccaacaaggccgtggtgtccctgtccaacggcgtgtccgtgctgacctccaaggtgctggatctgaagaactacatcaacaaccagctgctgcccatcgtgaaccagcagtcctgccggatcttcaacatcgagacagtgatcgagttccagcagaagaactcccggctgctggaaatcacccgcgagttctctgtgaatgccggcgtgaccacccccctgtccacctacatgctgaccaactccgagctgctgtccctgatcaacgacatgcccatcaccaacgaccagaaaaagctgatgtcctccaacgtgcagatcgtgcggcagcagagctactccatcatgtccattatcaaagaagaggtgctggcctacgtggtgcagctgcctatctacggcgtgatcgacaccccctgctggaagctgcacaccagccctctgtgcaccaccaacatcaaagagggctccaacatctgcctgaccagaaccgaccggggctggtactgtgacaacgctggctccgtctcattctttccacaagccgatacctgcaaggtgcagtccaaccgggtgttctgcgacaccatgaattctctgaccctgccctccgaagtgtctctgtgtaacaccgacatcttcaactctaagtacgactgcaagatcatgaccagcaagaccgatatctccagctctgtgatcacctccctgggcgccatcgtgtcctgctacggcaagaccaagtgcaccgcctccaacaagaaccggggcatcatcaagaccttctccaacggctgcgactacgtgtccaacaagggggtggacaccgtgtctgtgggcaacaccctgtactacgtgaacaagctggaagggaagaatctgtacgtgaagggcgagcccatcatcaactactacgaccccctggtgttccccagcgacgagttcgacgcctccatcagccaagtgaacgagaagatcaaccagtccctggccttcatccggaagtccgatgagctgctgcacaatgtgaacaccggcaagtccaccacaaatatcatgatcaccaccatcattatcgtgatcatcgtggtgctgctgagcctgatcgccatcggcctgctgctgtactgcaaggccaagaacacccccgtgaccctgagcaaggaccagctgtccggcatcaacaatatcgccttcagcaagtga

SEQ ID NO: 41 - DNA, 1725 bp, synthetic construct
  misc_feature 1..1725: note = Description of Artificial Sequence: Synthetic polynucleotide
  source 1..1725: mol_type = other DNA
atggaactgctgatccaccgttcctccgctatcttcctgaccctggctatcaacgctctgtacctgacctcctcccagaacatcaccgaggaattctaccagtctacctgctccgctgtgtcccgtggttacttctccgctctgcgtaccggctggtacacctccgtgatcaccatcgagctgtccaacatcactgagactaagtgcaacggcaccgacaccaaagtgaagctgatcaagcaagagctggacaagtacaagaacgctgtgaccgaactgcagctgctgatgcagaacacccccgctgctaacaaccgtgctcgtcgcgaagctccccagcacatgaactacaccatcaacaccaccaagaacctgaacgtgtccatctccaagaagcgcaagcgccgtttcctgggtttcctgctgggtgtcggttccgctatcgcttccggtatcgctgtctccaaggtgctgcacctcgagggcgaagtgaacaagatcaagaacgccctgctgtccaccaacaaggctgtggtgtccctgtccaacggtgtctccgtgctgacctccaaggtcctcgacctgaagaactacatcaacaaccagctgctgcccatcgtgaaccagcagtcctgccgtatcttcaacatcgagactgtgatcgagttccagcagaagaactcccgtctgctcgagatcacccgcgagttctccgtgaacgctggtgtcaccacccccctgtccacctacatgctgaccaactccgagctgctgtccctgatcaacgacatgcccatcaccaacgaccaaaagaagctgatgtcctccaacgtgcagatcgtgcgtcagcagtcttactccatcatgtccatcatcaaggaagaggtgctggcttacgtggtgcagctgcctatctacggtgtcatcgacaccccctgctggaagctgcacacctccccactgtgcaccaccaacatcaaggaaggttccaacatctgcctgacccgtaccgaccgtggctggtactgcgacaacgctggttccgtttcattcttcccacaagccgacacttgcaaggtgcagtccaaccgtgtgttctgcgacaccatgaactccctgactctgccctccgaggtgtccctctgcaacaccgacatcttcaactctaagtacgactgcaagatcatgacctctaagactgacatctcctccagcgtcatcacctccctgggtgctatcgtgtcctgctacggcaagaccaagtgcaccgcttccaacaagaaccgcggtatcatcaagaccttctccaacggttgcgactacgtgtccaacaagggcgtggacaccgtgtccgtgggcaacaccctgtactacgtgaacaagctcgagggcaagaacctctacgtgaagggcgagcctatcatcaactactacgaccccctggtgttcccatccgacgagttcgacgcttccatctcccaagtgaacgagaagatcaaccagtccctggctttcatccgcaagtccgacgagctgctccacaacgtgaacaccggcaagtccactactaacatcatgatcaccactatcatcatcgtgatcatcgtcgtgctgctgagcctgatcgctatcggcctgctgctgtactgcaaggctaagaacactcccgtgaccctgtctaaggaccagctgtccggtatcaacaacatcgccttcagcaagtaa

SEQ ID NO: 42 - DNA, 1725 bp, synthetic construct
  misc_feature 1..1725: note = Description of Artificial Sequence: Synthetic polynucleotide
  source 1..1725: mol_type = other DNA
atggaactgctgatccacagaagcagcgccatcttcctgaccctggccatcaacgccctgtacctgaccagcagccagaacatcaccgaggaattctaccagagcacctgtagcgccgtgtccagaggctacttcagcgccctgagaaccggctggtacaccagcgtgatcaccatcgagctgagcaacatcacagagacaaagtgcaacggcaccgacaccaaagtgaagctgatcaagcaggaactggacaagtacaagaacgccgtgaccgaactgcagctgctgatgcagaacacccctgccgccaacaacagagccagaagagaagccccccagcacatgaactacaccatcaacaccaccaagaacctgaacgtgtccatcagcaagaagaggaagagaagattcctgggcttcctgctgggcgtgggcagcgctatcgcttctggaatcgccgtgtctaaggtgctgcacctggaaggcgaagtgaacaagatcaagaacgctctgctgagcaccaacaaggccgtggtgtccctgagcaacggcgtgtccgtgctgacctccaaggtgctggatctgaagaactacatcaacaaccagctgctgcccatcgtgaaccagcagagctgcagaatcttcaacatcgagacagtgatcgagttccagcagaagaacagcaggctgctggaaatcacccgcgagttcagcgtgaacgctggcgtgaccacacccctgagcacctacatgctgaccaacagcgagctgctgtctctgatcaacgacatgcccatcaccaacgaccagaaaaagctgatgagcagcaacgtgcagatcgtgcggcagcagtcctacagcatcatgagcatcatcaaagaagaggtgctggcctacgtggtgcagctgcctatctacggcgtgatcgacaccccctgctggaagctgcacaccagccctctgtgcaccaccaacatcaaagagggcagcaacatctgcctgaccagaaccgacagaggctggtactgcgacaacgccggctccgtctcattctttccacaagccgacacatgcaaggtgcagagcaacagagtgttctgcgacaccatgaacagcctgacactgcccagcgaagtgtccctgtgtaacaccgacatcttcaactctaagtacgactgcaagatcatgaccagcaagaccgacatcagctcctccgtgatcacaagcctgggcgccatcgtgtcctgctacggcaagaccaagtgcaccgccagcaacaagaacaggggaatcatcaagaccttcagcaacggctgcgactacgtgtccaacaagggggtggacaccgtgtctgtgggcaacaccctgtactacgtgaacaagctggaagggaagaatctgtacgtgaagggcgagcccatcatcaactactacgaccccctggtgttccccagcgacgagttcgacgccagcatcagccaagtgaacgagaagatcaaccagagcctggccttcatcagaaagtccgatgagctgctgcacaatgtgaacaccggcaagtccacaacaaacatcatgatcaccaccatcattatcgtgatcatcgtggtgctgctgagcctgatcgccatcggcctgctgctgtactgcaaggccaagaacacacccgtgaccctgtccaaggaccagctgagcggcatcaacaatatcgccttctccaagtga

SEQ ID NO: 43 - DNA, 1707 bp, synthetic construct
  misc_feature 1..1707: note = Description of Artificial Sequence: Synthetic polynucleotide
  source 1..1707: mol_type = other DNA
atggaactgctgatcctgaaggccaacgccatcaccaccatcctgaccgccgtgaccttctgcttcgccagcggccagaacatcaccgaggaattctaccagagcacctgcagcgccgtgagcaagggctacctgagcgccctgcggaccggctggtacaccagcgtgatcaccatcgagctgtccaacatcaaagaaaacaagtgcaacggcaccgacgccaaagtgaagctgatcaagcaggaactggacaagtacaagaacgccgtgaccgagctgcagctgctgatgcagagcacccccgccaccaacaacagagccagaagagagctgccccggttcatgaactacaccctgaacaacgccaagaaaaccaacgtgaccctgagcaagaagagaaagagaagattcctgggcttcctgctgggcgtgggcagcgccattgccagcggcgtggccgtgtgcaaagtgctgcacctggaaggcgaagtgaacaagatcaagtccgccctgctgtccaccaacaaggccgtggtgtccctgagcaacggcgtgagcgtgctgaccttcaaggtgctggatctgaagaactacatcgacaagcagctgctgcccatcctgaacaagcagagctgcagcatcagcaacatcgagacagtgatcgagttccagcagaagaacaaccggctgctggaaatcacccgggagttcagcgtgaacgccggagtgaccacccccgtgtccacctacatgctgaccaacagcgagctgctgtccctgatcaatgacatgcccatcaccaacgaccagaaaaagctgatgagcaacaacgtgcagatcgtgcggcagcagagctactccatcatgtgcatcatcaaagaagaggtgctggcctacgtggtgcagctgcccctgtacggcgtgatcgacaccccctgctggaagctgcacaccagccccctgtgcacaaccaacaccaaagagggcagcaacatctgcctgacccggaccgaccggggctggtactgcgacaacgccggcagcgtgtccttctttccacaggccgagacatgcaaggtgcagagcaaccgggtgttctgcgacaccatgaacagcctgaccctgccctccgaagtgaacctgtgcaacgtggacatcttcaaccccaagtacgactgcaagatcatgacctccaagaccgacgtgtccagctccgtgatcacctccctgggcgccatcgtgtcctgctacggcaagaccaagtgcaccgccagcaacaagaacagaggcatcatcaagaccttcagcaacggctgcgactacgtgtccaataagggcgtggacaccgtgtccgtgggcaacacactgtactacgtgaataagcaggaaggcaagagcctgtacgtgaagggcgagcccatcatcaacttctacgaccccctggtgttccccagcgacgagttcgacgccagcatcagccaggtgaacgagaagatcaaccagagcctggccttcatcagaaagagcgacgaactgctgtccgccatcggcggctacatccccgaggcccccagagatggccaggcctacgtgcggaaggacggcgagtgggtgctgctgtctacatttctgggcggcctggtgcctagaggctctcaccaccaccatcaccacagcgcctggtcccacccccagttcgagaagtga

SEQ ID NO: 44 - DNA, 1707 bp, synthetic construct
  misc_feature 1..1707: note = Description of Artificial Sequence: Synthetic polynucleotide
  source 1..1707: mol_type = other DNA
atggaactgctgatcctgaaggccaacgccatcaccaccatcctgaccgccgtgaccttctgcttcgccagcggccagaacatcaccgaggaattctaccagagcacctgcagcgccgtgagcaagggctacctgagcgccctgcggaccggctggtacaccagcgtgatcaccatcgagctgtccaacatcaaagaaaacaagtgcaacggcaccgacgccaaagtgaagctgatcaagcaggaactggacaagtacaagaacgccgtgaccgagctgcagctgctgatgcagagcacccccgccaccaacaacagagccagaagagagctgccccggttcatgaactacaccctgaacaacgccaagaaaaccaacgtgaccctgagcaagaagagaaagagaagattcctgggcttcctgctgggcgtgggcagcgccattgccagcggcgtggccgtgtgcaaagtgctgcacctggaaggcgaagtgaacaagatcaagtccgccctgctgtccaccaacaaggccgtggtgtccctgagcaacggcgtgagcgtgctgaccagcaaggtgctggatctgaagaactacatcgacaagcagctgctgcccatcgtgaacaagcagagctgcagcatcagcaacatcgagacagtgatcgagttccagcagaagaacaaccggctgctggaaatcacccgggagttcagcgtgaacgccggagtgaccacccccgtgtccacctacatgctgaccaacagcgagctgctgtccctgatcaatgacatgcccatcaccaacgaccagaaaaagctgatgagcaacaacgtgcagatcgtgcggcagcagagctactccatcatgtgcatcatcaaagaagaggtgctggcctacgtggtgcagctgcccctgtacggcgtgatcgacaccccctgctggaagctgcacaccagccccctgtgcacaaccaacaccaaagagggcagcaacatctgcctgacccggaccgaccggggctggtactgcgacaacgccggcagcgtgtccttctttccacaggccgagacatgcaaggtgcagagcaaccgggtgttctgcgacaccatgaacagcctgaccctgccctccgaagtgaacctgtgcaacgtggacatcttcaaccccaagtacgactgcaagatcatgacctccaagaccgacgtgtccagctccgtgatcacctccctgggcgccatcgtgtcctgctacggcaagaccaagtgcaccgccagcaacaagaacagaggcatcatcaagaccttcagcaacggctgcgactacgtgtccaataagggcgtggacaccgtgtccgtgggcaacacactgtactacgtgaataagcaggaaggcaagagcctgtacgtgaagggcgagcccatcatcaacttctacgaccccctggtgttccccagcgacgagttcgacgccagcatcagccaggtgaacgagaagatcaaccagagcctggccttcatcagaaagagcgacgaactgctgtccgccatcggcggctacatccccgaggcccccagagatggccaggcctacgtgcggaaggacggcgagtgggtgctgctgtctacatttctgggcggcctggtgcctagaggctctcaccaccaccatcaccacagcgcctggtcccacccccagttcgagaagtga

SEQ ID NO: 45 - DNA, 1707 bp, synthetic construct
  misc_feature 1..1707: note = Description of Artificial Sequence: Synthetic polynucleotide
  source 1..1707: mol_type = other DNA
atggaactgctgatcctgaaggccaacgccatcaccaccatcctgaccgccgtgaccttctgcttcgccagcggccagaacatcaccgaggaattctaccagagcacctgcagcgccgtgagcaagggctacctgagcgccctgcggaccggctggtacaccagcgtgatcaccatcgagctgtccaacatcaaagaaaacaagtgcaacggcaccgacgccaaagtgaagctgatcaagcaggaactggacaagtacaagaacgccgtgaccgagctgcagctgctgatgcagagcacccccgccaccaacaacagagccagaagagagctgccccggttcatgaactacaccctgaacaacgccaagaaaaccaacgtgaccctgagcaagaagagaaagagaagattcctgggcttcctgctgggcgtgggcagcgccattgccagcggcgtggccgtgtccaaagtgctgcacctggaaggcgaagtgaacaagatcaagtccgccctgctgtccaccaacaaggccgtggtgtccctgagcaacggcgtgagcgtgctgaccttcaaggtgctggatctgaagaactacatcgacaagcagctgctgcccatcctgaacaagcagagctgcagcatcagcaacatcgagacagtgatcgagttccagcagaagaacaaccggctgctggaaatcacccgggagttcagcgtgaacgccggagtgaccacccccgtgtccacctacatgctgaccaacagcgagctgctgtccctgatcaatgacatgcccatcaccaacgaccagaaaaagctgatgagcaacaacgtgcagatcgtgcggcagcagagctactccatcatgagcatcatcaaagaagaggtgctggcctacgtggtgcagctgcccctgtacggcgtgatcgacaccccctgctggaagctgcacaccagccccctgtgcacaaccaacaccaaagagggcagcaacatctgcctgacccggaccgaccggggctggtactgcgacaacgccggcagcgtgtccttctttccacaggccgagacatgcaaggtgcagagcaaccgggtgttctgcgacaccatgaacagcctgaccctgccctccgaagtgaacctgtgcaacgtggacatcttcaaccccaagtacgactgcaagatcatgacctccaagaccgacgtgtccagctccgtgatcacctccctgggcgccatcgtgtcctgctacggcaagaccaagtgcaccgccagcaacaagaacagaggcatcatcaagaccttcagcaacggctgcgactacgtgtccaataagggcgtggacaccgtgtccgtgggcaacacactgtactacgtgaataagcaggaaggcaagagcctgtacgtgaagggcgagcccatcatcaacttctacgaccccctggtgttccccagcgacgagttcgacgccagcatcagccaggtgaacgagaagatcaaccagagcctggccttcatcagaaagagcgacgaactgctgtccgccatcggcggctacatccccgaggcccccagagatggccaggcctacgtgcggaaggacggcgagtgggtgctgctgtctacatttctgggcggcctggtgcctagaggctctcaccaccaccatcaccacagcgcctggtcccacccccagttcgagaagtga

SEQ ID NO: 46 - protein, 6 aa, synthetic construct
  REGION 1..6: note = Description of Artificial Sequence: Synthetic 6xHis tag
HHHHHH

SEQ ID NO: 47 - protein, 488 aa, Human respiratory syncytial virus
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELL